SEQUENCE LISTING (76 sequences)

SEQ ID NO: 1 - DNA, 74 bp, Artificial Sequence
  APC Gene Control Region Strand A
ggaccagggcgctccccattcccgtcgggagcccgccgattggctgggtgtgggcgcacgtgaccgacatgtgg

SEQ ID NO: 2 - DNA, 74 bp, Artificial Sequence
  APC Gene Control Region Strand B
ccacatgtcggtcacgtgcgcccacacccagccaatcggcgggctcccgacgggaatggggagcgccctggtcc

SEQ ID NO: 3 - DNA, 83 bp, Artificial Sequence
  RARB Gene Control Region Strand A
ccgagaacgcgagcgatccgagcagggtttgtctgggcaccgtcggggtaggatccggaacgcattcggaaggctttttgcaa

SEQ ID NO: 4 - DNA, 83 bp, Artificial Sequence
  RARB Gene Control Region Strand B
ttgcaaaaagccttccgaatgcgttccggatcctaccccgacggtgcccagacaaaccctgctcggatcgctcgcgttctcgg

SEQ ID NO: 5 - DNA, 47 bp, Artificial Sequence
  APC Cloning Methylated Extension Template (top)
gaaccaaaacgctccccattcccgtcgaaaacccgccgattaactaa

SEQ ID NO: 6 - DNA, 47 bp, Artificial Sequence
  APC Cloning Methylated Extension Template (bottom)
ttatatgtcggttacgtgcgtttatatttagttaatcggcgggtttt

SEQ ID NO: 7 - DNA, 52 bp, Artificial Sequence
  APC Cloning Unmethylated Extension Template (top)
ctaaatacaaaccaaaacactccccattcccatcaaaaacccaccaattaac

SEQ ID NO: 8 - DNA, 52 bp, Artificial Sequence
  APC Cloning Unmethylated Extension Template (bottom)
agttatatgttggttatgtgtgtttatatttagttaattggtgggtttttga

SEQ ID NO: 9 - DNA, 19 bp, Artificial Sequence
  APC QPCR Unconverted Primer (forward)
ggaccagggcgctccccat

SEQ ID NO: 10 - DNA, 27 bp, Artificial Sequence
  APC QPCR Unconverted Primer (reverse)
ccacatgtcggtcacgtgcgcccacac

SEQ ID NO: 11 - DNA, 22 bp, Artificial Sequence
  APC QPCR Unconverted Probe
cccgtcgggagcccgccgattg

SEQ ID NO: 12 - DNA, 19 bp, Artificial Sequence
  APC QPCR Methylated Primer (forward)
gaaccaaaacgctccccat

SEQ ID NO: 13 - DNA, 27 bp, Artificial Sequence
  APC QPCR Methylated Primer (reverse)
ttatatgtcggttacgtgcgtttatat

SEQ ID NO: 14 - DNA, 22 bp, Artificial Sequence
  APC QPCR Methylated Probe
cccgtcgaaaacccgccgatta

SEQ ID NO: 15 - DNA, 27 bp, Artificial Sequence
  APC QPCR Unmethylated Primer (forward)
ctaaatacaaaccaaaacactccccat

SEQ ID NO: 16 - DNA, 29 bp, Artificial Sequence
  APC QPCR Unmethylated Primer (reverse)
agttatatgttggttatgtgtgtttatat

SEQ ID NO: 17 - DNA, 22 bp, Artificial Sequence
  APC QPCR Unmethylated Probe
cccatcaaaaacccaccaatta

SEQ ID NO: 18 - DNA, 20 bp, Artificial Sequence
  APC Cloning Primer (forward)
actgccatcaacttccttgc

SEQ ID NO: 19 - DNA, 20 bp, Artificial Sequence
  APC Cloning Primer (reverse)
acctaccccatttccgagtc

SEQ ID NO: 20 - DNA, 19 bp, Artificial Sequence
  RARB Cloning Primer (forward)
caattcaatctttcattct

SEQ ID NO: 21 - DNA, 26 bp, Artificial Sequence
  RARB Cloning Primer (reverse)
ttgcaaaaagccttccgaatgcgttc

SEQ ID NO: 22 - DNA, 50 bp, Artificial Sequence
  RARB Cloning Methylarted Extension Template (top)
agaacgcgagcgattcgagtagggtttgtttgggtatcgtcggggtagga

SEQ ID NO: 23 - DNA, 50 bp, Artificial Sequence
  RARB Cloning Methylarted Extension Template (bottom)
tacaaaaaaccttccgaatacgttccgaatcctaccccgacgatacccaa

SEQ ID NO: 24 - DNA, 52 bp, Artificial Sequence
  RARB Cloning Unmethylarted Extension Template (top)
ttgagaatgtgagtgatttgagtagggtttgtttgggtattgttggggtagg

SEQ ID NO: 25 - DNA, 52 bp, Artificial Sequence
  RARB Cloning Unmethylarted Extension Template (bottom)
ttacaaaaaaccttccaaatacattccaaatcctaccccaacaatacccaaa

SEQ ID NO: 26 - DNA, 25 bp, Artificial Sequence
  RARB QPCR Unconverted Primer (forward)
ccgagaacgcgagcgatccgagcag

SEQ ID NO: 27 - DNA, 26 bp, Artificial Sequence
  RARB QPCR Unconverted Primer (reverse)
ttgcaaaaagccttccgaatgcgttc

SEQ ID NO: 28 - DNA, 24 bp, Artificial Sequence
  RARB QPCR Unconverted Probe
atcctaccccgacggtgcccagac

SEQ ID NO: 29 - DNA, 22 bp, Artificial Sequence
  RARB QPCR Methylated Primer (forward)
agaacgcgagcgattcgagtag

SEQ ID NO: 30 - DNA, 24 bp, Artificial Sequence
  RARB QPCR Methylated Primer (reverse)
tacaaaaaaccttccgaatacgtt

SEQ ID NO: 31 - DNA, 24 bp, Artificial Sequence
  RARB QPCR Methylated Probe
atcctaccccgacgatacccaaac

SEQ ID NO: 32 - DNA, 25 bp, Artificial Sequence
  RARB QPCR Unmethylated Primer (forward)
ttgagaatgtgagtgatttgagtag

SEQ ID NO: 33 - DNA, 26 bp, Artificial Sequence
  RARB QPCR Unmethylated Primer (reverse)
ttacaaaaaaccttccaaatacattc

SEQ ID NO: 34 - DNA, 26 bp, Artificial Sequence
  RARB QPCR Unmethylated Probe
aaatcctaccccaacaatacccaaac

SEQ ID NO: 35 - DNA, 57 bp, Artificial Sequence
  GSTP1 Cloning Methylated Extension Template (top)
ttcggggtgtagcggtcgtcgggttggggtcggcgggagttcgcgggattttttaga

SEQ ID NO: 36 - DNA, 56 bp, Artificial Sequence
  GSTP1 Cloning Methylated Extension Template (bottom)
gccccaatactaaatcacgacgccgaccgctcttctaaaaaatcccgcgaactccc

SEQ ID NO: 37 - DNA, 58 bp, Artificial Sequence
  GSTP1 Cloning Unmethylated Extension Template (top)
gatgtttggggtgtagtggttgttgggttggggttggtgggagtttgtgggatttttt

SEQ ID NO: 38 - DNA, 58 bp, Artificial Sequence
  GSTP1 Cloning Unmethylated Extension Template (bottom)
ccaccccaatactaaatcacaacaccaaccactcttctaaaaaatcccacaaactccc

SEQ ID NO: 39 - DNA, 20 bp, Artificial Sequence
  GSTP1 QPCR Methylated Primer (forward)
ttcggggtgtagcggtcgtc

SEQ ID NO: 40 - DNA, 22 bp, Artificial Sequence
  GSTP1 QPCR Methylated Primer (reverse)
gccccaatactaaatcacgacg

SEQ ID NO: 41 - DNA, 23 bp, Artificial Sequence
  GSTP1 QPCR Methylated Probe
taaaaaatcccgcgaactcccgc

SEQ ID NO: 42 - DNA, 24 bp, Artificial Sequence
  GSTP1 QPCR Unmethylated Primer (forward)
gatgtttggggtgtagtggttgtt

SEQ ID NO: 43 - DNA, 24 bp, Artificial Sequence
  GSTP1 QPCR Unmethylated Primer (reverse)
ccaccccaatactaaatcacaaca

SEQ ID NO: 44 - DNA, 22 bp, Artificial Sequence
  GSTP1 QPCR Unmethylated Probe
aaaaatcccacaaactcccacc

SEQ ID NO: 45 - DNA, 48 bp, Artificial Sequence
  RASSF1 Cloning Methylated Extension Template (top)
cgcttgaagtcggggttcgttttgtggtttcgttcggttcgcgtttgt

SEQ ID NO: 46 - DNA, 48 bp, Artificial Sequence
  RASSF1 Cloning Methylated Extension Template (bottom)
cccgtacttcgctaactttaaacgctaacaaacgcgaaccgaacgaaa

SEQ ID NO: 47 - DNA, 48 bp, Artificial Sequence
  RASSF1 Cloning Unmethylated Extension Template (top)
gtgttgaagttggggtttgttttgtggttttgtttggtttgtgtttgt

SEQ ID NO: 48 - DNA, 48 bp, Artificial Sequence
  RASSF1 Cloning Unmethylated Extension Template (bottom)
cccatacttcactaactttaaacactaacaaacacaaaccaaacaaaa

SEQ ID NO: 49 - DNA, 18 bp, Artificial Sequence
  RASSF1 QPCR Methylated Primer (forward)
gcgttgaagtcggggttc

SEQ ID NO: 50 - DNA, 24 bp, Artificial Sequence
  RASSF1 QPCR Methylated Primer (reverse)
cccgtacttcgctaactttaaacg

SEQ ID NO: 51 - DNA, 24 bp, Artificial Sequence
  RASSF1 QPCR Methylated Probe
acaaacgcgaaccgaacgaaacca

SEQ ID NO: 52 - DNA, 18 bp, Artificial Sequence
  RASSF1 QPCR Unmethylated Primer (forward)
gtgttgaagttggggttt

SEQ ID NO: 53 - DNA, 24 bp, Artificial Sequence
  RASSF1 QPCR Unmethylated Primer (reverse)
cccatacttcactaactttaaaca

SEQ ID NO: 54 - DNA, 24 bp, Artificial Sequence
  RASSF1 QPCR Unmethylated Probe
acaaacacaaaccaaacaaaacca

SEQ ID NO: 55 - DNA, 48 bp, Artificial Sequence
  PSA Cloning Primer (upper)
cctcacagctgcccactgcatcaggaacaaaagcgtgatcttgctggg

SEQ ID NO: 56 - DNA, 47 bp, Artificial Sequence
  PSA Cloning Primer (lower)
gatgaaacaggctgtgccgacccagcaagatcacgcttttgttcctg

SEQ ID NO: 57 - DNA, 19 bp, Artificial Sequence
  PSA RT-QTPCR Primer (forward)
gatgaaacaggctgtgccg

SEQ ID NO: 58 - DNA, 20 bp, Artificial Sequence
  PSA RT-QTPCR Primer (reverse)
cctcacagctgcccactgca

SEQ ID NO: 59 - DNA, 27 bp, Artificial Sequence
  PSA RT-QTPCR Probe
caggaacaaaagcgtgatcttgctggg

SEQ ID NO: 60 - DNA, 61 bp, Artificial Sequence
  PCA3DD3 Cloning Primer (upper)
cacaggaagcacaaaaggaagcacagagatccctgggagaaatgcccggccgccatcttgg

SEQ ID NO: 61 - DNA, 60 bp, Artificial Sequence
  PCA3DD3 Cloning Primer (lower)
acaagcgggaccaggcacagggcgaggctcatcgatgacccaagatggcggccgggattt

SEQ ID NO: 62 - DNA, 24 bp, Artificial Sequence
  PCA3DD3 RT-QPCR Primer (forward)
agcacaaaaggaagcacagagatc

SEQ ID NO: 63 - DNA, 20 bp, Artificial Sequence
  PCA3DD3 RT-QPCR Primer (reverse)
acaagcgggaccaggcacag

SEQ ID NO: 64 - DNA, 24 bp, Artificial Sequence
  PCA3DD3 RT-QPCR Probe
catcgatgacccaagatggcggcc

SEQ ID NO: 65 - DNA, 52 bp, Artificial Sequence
  TMPRSS2ERG Cloning Primer (upper)
gggagcgccgcctggagcgcggcaggaagccttatcagttgtgagtgaggac

SEQ ID NO: 66 - DNA, 50 bp, Artificial Sequence
  TMPRSS2ERG Cloning Primer (lower)
ttccgtaggcacactcaaacaacgactggtcctcactcacaactgataag

SEQ ID NO: 67 - DNA, 18 bp, Artificial Sequence
  TMPRSS2ERG RT-QPCR Primer (forward)
ggagcgccgcctggagcg

SEQ ID NO: 68 - DNA, 22 bp, Artificial Sequence
  TMPRSS2ERG RT-QPCR Primer (reverse)
tccgtaggcacactcaaacaac

SEQ ID NO: 69 - DNA, 20 bp, Artificial Sequence
  TMPRSS2ERG RT-QPCR Probe
cagttgtgagtgaggaccag

SEQ ID NO: 70 - DNA, 19 bp, Artificial Sequence
  hTERT RT-QPCR Primer (forward)
acggcgacatggagaacaa

SEQ ID NO: 71 - DNA, 21 bp, Artificial Sequence
  hTERT RT-QPCR Primer (reverse)
cactgtcttccgcaagttcac

SEQ ID NO: 72 - DNA, 29 bp, Artificial Sequence
  hTERT RT-QPCR Probe
ctcctgcgttggtggatgatttcttgttg

SEQ ID NO: 73 - DNA, 18 bp, Artificial Sequence
  GADPH Cloning and RT-QPCR Primer (forward)
gaaggtgaaggtcggagt

SEQ ID NO: 74 - DNA, 20 bp, Artificial Sequence
  GADPH Cloning Primer (reverse)
gaagatggtgatgggatttg

SEQ ID NO: 75 - DNA, 20 bp, Artificial Sequence
  GADPH RT-QPCR Primer (reverse)
gaagatggtgatgggatttc

SEQ ID NO: 76 - DNA, 20 bp, Artificial Sequence
  GADPH RT-QPCR Probe
caagcttcccgttctcagcc

(19) United States
(12) Patent Application Publication
(10) Pub. No.: US 2009/0263799 A1
(43) Pub. Date: Oct. 22, 2009

(54) ASSAY FOR PROSTATE CANCER

(76) Inventors: Steven Smith, Los Angeles, CA (US); Jarrod Clark, Ontario, CA (US); Kristofer Munson, Glendora, CT (US)

Correspondence Address: PERKINS COIE LLP, POST OFFICE BOX 1208, SEATTLE, WA, 98111-1208, US

(21) Appl. No.: 12/327,788

(22) Filed: Dec. 3, 2008

Related U.S. Application Data
(60) Provisional application No. 61/005,376, filed on Dec. 3, 2007.

(51) Int. Cl. C12Q 1/68

(52) U.S. Cl. 435/6

(57) ABSTRACT

In certain embodiments, a method for detecting a prostate proliferative cell disorder, a prostate cancer or a prostate tumor and/or categorizing Gleason's Sum of the tumors includes performing a digital rectal examination on a subject; obtaining one or more expressed prostatic secretion (EPS) samples from the subject; measuring PSA levels in the EPS; and measuring a biomarker, wherein the biomarker is TMPRSS2:ERG fusion RNA. Optionally, the biomarker may be methylated copies of GSTPI, APC, RARB and/or RASSFI DNA or PCA3 RNA. A kit for performing any of the above embodiments is also contemplated.

### PRIORITY CLAIM

This application claims the benefit of U.S. Provisional Application Ser. No. 61/005,376, filed Dec. 3, 2007, which is incorporated herein by reference.

### GOVERNMENT INTEREST

The present invention was made with government support under City of Hope's Cancer Center Support Grant 5P30CA033572-22 and by grant 5R01-CA102521-01 to S.S.S. from the U.S. National Cancer Institute of the National Institutes of Health. Other support for data analysis was provided by the NCI's Early Detection Research Network U01-CA86368 to Ziding Feng (Seattle). The government has certain rights in the present invention.

### BACKGROUND

The evolutionary biology of tumorigenesis involves the natural selection of spontaneous or carcinogen induced genetic and epigenetic variants. Current evidence suggests that the disruption of epigenetic control systems like DNA methylation is associated with both altered gene expression (Robertson, 2000; Rountree, 2000; Macleod, 1994; Brandeis, 1994) and genetic instability (Smith, 1991; Smith, 1999) during tumorigenesis. Thus, epigenetic biomarkers like changes in DNA methylation state and changes in gene expression are expected to co-evolve with genetic biomarkers such as gene rearrangements during tumorigenesis.

With promoter hypermethylation of DNA, the actual shutdown of gene expression generally requires downstream processes like the binding of methylated DNA binding proteins that are often absent in prostate cancer cells (Carro, 2004; Patra, 2003). Thus compensating mutations can produce leaky expression of tumor suppressors (in preparation), thereby diminishing the value of hypermethylation as a biomarker. Even so, hypermethylation of several genes including GSTPI, APC, RARB and RASSFI has been shown to distinguish normal prostate cells from prostate cancer cells in a variety of settings (Crocitto, 2004; Goessl, 2001; Gonzalgo, 2004; Gonzalgo, 2003; Hoque, 2005; Jeronimo, 2002).

In mammals, DNA methylation patterns are known to be important hallmarks of both cell type and cellular history. Patterns of methylation are maintained in a given cell lineage (Razin, 1980) but alterations in these patterns are associated with changes in gene expression (Razin, 1980), cellular differentiation (Chen, 2005), gene rearrangement, telomere shortening, DNA damage, viral integration (Smith, 1999; Smith, 2000), carcinogenesis (Jones, 2005; Baylin, 2006) and aging (Issa, 2000). Given these associations, a good deal of effort has been invested in developing methods that can detect qualitative and quantitative changes in methylation patterns as biomarkers of these processes.

The use of methylation-sensitive restriction enzymes was employed early on (Waalwijk, 1978) as a qualitative indicator of methylation status, and methods of this type continue to be developed (Xiong, 1997). Other early techniques employed hydrazine (Church, 1984; Saluz, 1989; Pfeifer, 1989) or potassium permanganate (Rein, 1997) DNA modification for genomic sequencing. However, since its introduction (Frommer, 1992) the use of bisulfite-treated DNA as a means of distinguishing methylated cytosine from unmethylated cytosine in genomic applications has come into general use in the field. Certain artifacts can be avoided with highly purified DNA (Warnecke, 2002), however, the nature of the bisulfite reaction itself presents additional problems.

Bisulfite-mediated deamination of cytosine in DNA occurs only at low pH, in a solution that is effectively dilute sulfurous acid (Hayatsu, 1970; Hayatsi, 1970; Shiraishi, 2004; Wang 1980). Chemically this is required because of the low pKa of cytosine and the necessity for protonation of the N3 ring nitrogen in order to produce uracil or thymine from cytosine or 5-methylcytosine, respectively. The reaction rate for cytosine to uracil is much faster than the reaction rate for 5-methylcytosine to thymine, making it possible to detect 5-methylcytosines in biological samples as cytosine moieties that survive treatment with mild sulfurous acid. Superimposed on these reactions (FIG. 1) is the tendency for the glycosyl bond to undergo hydrolysis at sites of protonated bases in DNA coupled with chain breakage (Suzuki, 1994). In this case, base loss is rapidly followed by conversion to the aldose and b-elimination resulting in chain breakage (Grunau, 2001).

Many existing approaches to the analysis of methylation patterns now rely on bisulfite-treated DNA followed by PCR amplification. Out of necessity, the use of this reagent requires its removal prior to PCR amplification. This desulfonation step is generally accomplished by exposing the DNA product to mild base coupled with binding to and elution from a matrix. Moreover, most work in cancer research has shown that no single gene can suffice for accurate prediction of clinical diagnosis or outcome. Thus, one is faced with the practical limitations associated with testing multiple genes superimposed on the limitations placed on these analyses by specimen size. While this has led to the introduction of multiplex PCR, mass spectroscopic systems and multigene array systems, the fundamental reliance on the bisulfite-mediated deamination of cytosine and subsequent purification of the product remains central to each of these techniques.

While at least one report of the extent and rapidity of the degradation of DNA by bisulfite has appeared (Grunau, 2001), the extent of this side reaction has not been fully appreciated in the studies of DNA methylation. Moreover, studies on the effect of this side reaction on the MS-QPCR have not been reported.

Studies on the in vitro evolution of nucleic acids have demonstrated that they can adopt an almost unlimited number of conformations (Mills, 1967; Irvine, 1991; Paul, 2006; Wilson, 1999). For modern DNA, the selective pressures imposed by the transmission of genetic information through DNA replication require the presence of two Watson-Crick-complementary DNA strands in most living organisms. These constraints confine the conformation space occupied by the DNA of modern organisms (Smith, 1999) and make the Watson-Crick-paired duplex the dominant DNA secondary structure isolated from living things. Tertiary structures involving the Watson-Crick paired duplex include linear and bent duplexes, supercoils, and various recombination intermediates like the Holliday structure.

High conformation space sequences, on the other hand, are often characterized by high G+C content and/or segregation of purines and pyrimidines to different strands possessing Watson-Crick homology. Sequences with high conformation spaces tend to promote clastic mutations (Liu, 1995; Smith, 1994; Mitas, 1995; Chen, 1995; Mitas, 1995; Kovtun, 2001; Gacy, 1995; Darlow, 1998; Darlow, 1998; Raghavan, 2005). Even so G+C-rich islands mark the promoter regions of about half of the known genes in the human genome (Antequera, 1993), and promoter sequences are generally found to have a higher G+C content than their surroundings (Trinklein, 2003). Many of these sequences have been reported to adopt non-B DNA structures (Sun, 2005; Lew, 2000; Haga, 2004; Ackerman, 1993; Simonsson, 1998) that may function along with DNA methylation (Smith, 1994; Smith, 1997) and histone modification in the elaboration of stable epigenetic transitions.

Promoter sequences of, e.g., the APC, GSTPI and RARB genes are sequences of this type. Each is prone to de novo methylation at CG sites during carcinogenesis (Fackler, 2004; Harden, 2003; Dulaimi, 2004), and each has the potential for the formation of unusual DNA structures and single-strand conformers. The capacity of these sequences to interfere with biological analysis (e.g. DNA sequencing and PCR amplification) is well known.

In addition to the specific hypermethylation of tumor suppressor genes, an overall hypomethylation of DNA can be observed in tumor cells. This decrease in global methylation can be detected early, well before the development of frank tumor formation. A correlation between hypomethylation and increased gene expression has been determined for many oncogenes.

Prostate cancer is the most common malignancy among men in the United States (˜200,000 new cases per year), and the sixth leading cause of male cancer-related deaths worldwide (˜204,000 per year). Approximately 16% of men between the ages of 60 and 79 have this disease. Benign prostate hypertrophy is present in about 50% of men aged 50 or above, and in 95% of men aged 75 or above.

Current guidelines for prostate cancer screening have been suggested by the American Cancer Society and are as follows: At 50 years of age, health care professionals should offer a blood test for prostate specific antigen (PSA) and perform a digital rectal exam (DRE). It is recommended that high risk populations, such as African Americans and those with a family history of prostate disease, should begin screening at 45 years of age. Men without abnormal prostate pathology generally have a PSA level in blood below 4 ng/ml. PSA levels between 4 ng/ml and 10 ng/ml have a 25% chance of having prostate cancer. Numerous methods exist for measuring PSA (age-adjusted PSA cut-points, percent-free PSA, PSA velocity, PSA density, PSA doubling time, etc.), and each has an associated accuracy for detecting the presence of cancer. Yet, even with the minor improvements in detection, and the reported drops in mortality associated with screening, the frequency of false positives remains high. Reduced specificity results in part from increased blood PSA associated with BPH, and prostatis. It has also been estimated that up to 45% of prostate biopsies under currrent guidelines are falsely negative, resulting in decreased sensitivity even with biopsy.

According to the Prostate Cancer Institute, there are 4 major diagnostic tools for detecting prostate cancer. They fall into categories of those that screen for the disease and those that assist in determining the stage of the disease when found. These screening tests include: the Prostate-specific antigen test (PSA test), Digital rectal exam (DRE), Transrectal ultrasound, and prostate biopsy. Staging of an identified prostate cancer includes identifying whether the cancer is confined to the prostate, has grown beyond the prostate, and has spread (if so, where it has spread). Staging of prostate cancer disease is crucial to determining the best treatment.

While the PSA test is more sensitive than the digital rectal examination for detecting prostate cancer, some early cases of prostate cancer may be missed by the PSA screening cut-point of 4.0 ug/L.

Regarding regional hypomethylation, gene rearrangements and gene expression appear to be potentiated and can occur within a given hypomethylated region. Moreover, RNA expression is often associated with promoter hypomethylation (Jones, 2007), however, expression levels tend to depend on the selective advantage offered to the tumor, and the value of over-expression of tumor markers depends on the ease and uniqueness of the detection method. PCA3^{DD3} is a well-characterized biomarker that is over-expressed in prostate cancer (Reynolds, 2007; Marks, 2007; van Gils, 2007). Currently the selective advantage that it confers on the cancer cell is unknown. A second well-characterized expression marker comprises the various chromosome 21 fusions that can occur in prostate cancer between TMPRSS2 and ERG (Perner, 2006) and TMPRSS2 and ETS (Tomlins, 2005). Among these fusions several of the subtypes of the TMPRSS2:ERG (Laxman, 2006) have been detected in multiple chromosomal copies in prostate cancer cells (Attard, 2007). Ultimately, the efficacy of each of these biomarkers will depend on the ease and reliability of detection of each in a given biospecimen. Molecular markers of prostate cancer would offer the advantage that they can be used to efficiently analyze even small samples of tissue, including samples whose tissue architecture is not intact. While several genes have been studied with respect to differential expression among benign hyperplasia of the prostate and different grades of prostate cancer, no single marker has yet been shown to be sufficient to detect prostate cancer in a clinical setting.

Aberrant genetic methylation in prostate cancer has been observed in several genes including Gstp1, AR, p16 (CDKN2a/INK4a), CD44, CDH1. Genome-wide hypomethylation for example of the LINE-1 repetitive element has also been associated with tumor progression (Santourlidis, S., et al., Prostate 39:166-74, 1999). However, use of these genes as alternative or supplemental diagnostic markers in a commercial setting has not been enabled. The application of differentially methylated genes to clinically utilizable platforms requires much further investigation into the sensitivity and specificity of the genes. For example, in the case of the gene CD44, a known metastasis suppressor, downregulation was associated with hypermethylation. However the use of this gene as a commercially available marker was not enabled, because it was also methylated in normal tissues (see V is, et al., Mol. Urol. 5:199-203, 2001).

Therefore, there exists a need for a method of diagnosing prostate cell proliferative disorders such as prostate cancer with improved sensitivity, specificity and/or predictive value. The invention addresses the longfelt need for novel means for the early diagnosis of prostate cell proliferative disorders, in particular for the detection of prostate cancer, prostate carcinoma and prostate neoplasms.

### SUMMARY

The present invention provides novel methods and nucleic acids for the detection of and/or differentiation between prostate cell proliferative disorders.

The invention provides a panel of genes, RNA sequences, genomic sequences and/or regulatory regions, the expression levels being indicative of the presence of prostate cell proliferative disorders or features thereof. In particular the methylation status of CpG positions are indicative of the presence of prostate cell proliferative disorders or differentiation between such disorders. Preferred selections and combinations of genes are provided, the methylation analysis of which enable the detection of prostate cell proliferative disorders.

The method includes the diagnosis of prostate cell proliferative disorders; (preferably prostate cancer, prostate carcinoma and/or neoplasms) it is preferred that said genes and/or sequences are selected from the group consisting of: PSA RNA; TMPRSS2:ERG RNA; copy number of GSTPI, APC, RARB or RASSFI DNA; TMPRSS2:ERG Type III or VI fusion RNA; and methylation status of DNA sequences encoding GSTPI, APC, RARB or RASSFI or PCA3.

It is preferred that the prostate cell proliferative disorder is a prostate cancer, prostate carcinoma or prostate neoplasm. In further embodiments the invention provides methods and nucleic acids for the differentiation between non-cancerous types of prostate tissue (including benign prostatic hyperplasia aka “BPH” and normal) from prostate carcinoma.

In other embodiments the invention provides methods and nucleic acids for the differentiation of prostate cancer from normal prostate tissue, tissues originating from other tissues and BPH. In further embodiments the invention provides methods and nucleic acids for the differentiation of prostate cancer from other tissues in a biological sample obtained by a non-invasive means.

Preferably the prostate cell proliferative disorder is a prostate cancer, prostate carcinoma or prostate neoplasm. In further embodiments the invention provides methods and nucleic acids for the differentiation between non-cancerous types of prostate tissue (including BPH and normal) from prostate carcinoma. In further embodiments the invention provides methods and nucleic acids for the differentiation of prostate cancer from normal prostate tissue, tissues originating from other tissues and BPH. In certain embodiments, a method of detecting or grading prostate tumors or cancer comprises obtaining one or more expressed prostatic secretion (EPS) samples from a subject and measuring the level of a biomarker EPS.

The present invention provides a method for determining genetic and/or epigenetic parameters of genomic DNA. The method has utility for the improved detection of and/or differentiation between prostate cell proliferative disorders. Although methylation assays for the detection of prostate cancer are known there is currently no molecular classification system for the detection of prostate cell proliferative disorders, nor one that accurately differentiates benign conditions from prostate carcinomas and neoplasms.

The biosample source may be from any suitable source. Preferably, the source of the sample is selected from the group consisting of cells or cell lines, histological slides, biopsies, paraffin-embedded tissue, bodily fluids, ejaculate, urine, blood, and combinations thereof. Preferably, the source is biopsies, prostatic fluid, bodily fluids, ejaculate, urine, or blood.

Preferably, distinguishing between methylated and non methylated CpG dinucleotide sequences within the target sequence comprises methylation state-dependent conversion or non-conversion of at least one such CpG dinucleotide sequence to the corresponding converted or non-converted dinucleotide sequence within a sequence selected from the group consisting of PSA RNA; TMPRSS2:ERG RNA; copy number of GSTPI, APC, RARB or RASSFI DNA; TMPRSS2:ERG Type III or VI fusion RNA; and methylation status of DNA sequences encoding GSTPI, APC, RARB or RASSFI or PCA3, and contiguous regions thereof corresponding to the target sequence.

Additional embodiments provide a method for the detection of and/or differentiation between prostate cell proliferative disorders, comprising: obtaining a biological sample having subject genomic DNA; extracting the genomic DNA; treating the genomic DNA, or a fragment thereof, with one or more reagents to convert 5-position unmethylated cytosine bases to uracil or to another base that is detectably dissimilar to cytosine in terms of hybridization properties; contacting the treated genomic DNA, or the treated fragment thereof, with an amplification enzyme and at least two primers comprising, in each case a contiguous sequence at least 9 nucleotides in length that is complementary to, or hybridizes under moderately stringent or stringent conditions to a sequence corresponding to a gene encoding a biomarker selected from the group consisting PSA RNA; TMPRSS2:ERG RNA; copy number of GSTPI, APC, RARB or RASSFI DNA; TMPRSS2:ERG Type III or VI fusion RNA; and methylation status of DNA sequences encoding GSTPI, APC, RARB or RASSFI or PCA3, and complements thereof, wherein the treated DNA or the fragment thereof is either amplified, or is not amplified; and determining, based on a presence or absence of, or on a property of said amplified DNA, the methylation state of at least one CpG dinucleotide sequence selected from a sequence encoding a biomarker selected from the group consisting of PSA RNA; TMPRSS2:ERG RNA; copy number of GSTPI, APC, RARB or RASSFI DNA; TMPRSS2:ERG Type III or VI fusion RNA; and methylation status of DNA sequences encoding GSTPI, APC, RARB or RASSFI or PCA3, or an average, or a value reflecting an average methylation state of a plurality of CpG dinucleotide sequences thereof.

Further embodiments provide a method for the detection of and/or differentiation between prostate cell proliferative disorders, comprising: obtaining a biological sample having subject genomic DNA; extracting the genomic DNA; contacting the genomic DNA, or a fragment thereof, comprising one or more sequences encoding a biomarker selected from the group consisting of PSA RNA; TMPRSS2:ERG RNA; copy number of GSTPI, APC, RARB or RASSFI DNA; TMPRSS2:ERG Type III or VI fusion RNA; and methylation status of DNA sequences encoding GSTPI, APC, RARB or RASSFI or PCA3 or a sequence that hybridizes under stringent conditions thereto, with one or more methylation-sensitive restriction enzymes, wherein the genomic DNA is either digested thereby to produce digestion fragments, or is not digested thereby; and determining, based on a presence or absence of, or on property of at least one such fragment, the methylation state of at least one CpG dinucleotide sequence of one or more sequences encoding a biomarker selected from the group consisting of PSA RNA; TMPRSS2:ERG RNA; copy number of GSTPI, APC, RARB or RASSFI DNA; TMPRSS2:ERG Type III or VI fusion RNA; and methylation status of DNA sequences encoding GSTPI, APC, RARB or RASSFI or PCA3 or an average, or a value reflecting an average methylation state of a plurality of CpG dinucleotide sequences thereof. The digested or undigested genomic DNA may be amplified prior to said determining. Additional embodiments provide novel genomic and chemically modified nucleic acid sequences, as well as oligonucleotides and/or PNA-oligomers for analysis of cytosine methylation patterns within sequences encoding a biomarker selected from the group consisting of PSA RNA; TMPRSS2:ERG RNA; copy number of GSTPI, APC, RARB or RASSFI DNA; TMPRSS2:ERG Type III or VI fusion RNA; and methylation status of DNA sequences encoding GSTPI, APC, RARB or RASSFI or PCA3.

In certain embodiments, a method of detecting prostate tumors or cancer and/or categorizing Gleason's Sum of the prostate tumors comprises performing a digital rectal examination on a subject; measuring or detecting the level of PSA in the subject's serum; obtaining one or more expressed prostatic secretion (EPS) samples from the subject; measuring or detecting PSA levels in the EPS; and measuring or detecting a biomarker in the EPS, wherein the biomarker is TMPRSS2:ERG (e.g., TMPRSS2:ERG RNA or TMPRSS2:ERG fusion RNA).

In certain embodiments, a method of detecting prostate tumors or cancer comprises performing a digital rectal examination on a subject, measuring or detecting the level of PSA in the subject's serum and obtaining one or more expressed prostatic secretion (EPS) samples from the subject. The following analyses of the samples are performed: (a) measuring or detecting PSA levels in the EPS; and (b) measuring or detecting methylated copies of GSTPI, APC, RARB and/or RASSFI by performing PCR, wherein prior to amplifying the nucleic acid (e.g., DNA or RNA), the nucleic acid is treated with bisulfite without being previously denatured. In certain embodiments, the DNA may be previously denatured.

In certain embodiments, a method of detecting prostate tumors or cancer comprises performing a digital rectal examination on a subject; measuring or detecting the level of PSA in the subject's serum; obtaining one or more expressed prostatic secretion (EPS) samples from the subject; measuring or detecting PSA levels in the EPS; and measuring or detecting a biomarker in the EPS, wherein the biomarker is PCA3 (e.g., PCA3 RNA). A kit for performing any of the above embodiments which includes combinations of the various components and ingredients described herein is also included.

In certain embodiments, a method of detecting prostate tumors or cancer comprises performing a digital rectal examination on a subject, measuring or detecting PSA in the subject's serum; obtaining one or more expressed prostatic secretion (EPS) samples from the subject; measuring or detecting one or more of the markers described in [0014-0016] without measuring PSA RNA in the EPS.

The method and nucleic acids according to the invention are used for detection of, screening of populations for, differentiation between, monitoring of, and detection and monitoring of prostate cell proliferative disorders.

### BRIEF DESCRIPTION OF THE DRAWINGS

FIG. 1. Sulfurous acid (bisulfite)-mediated deamination and degradation of DNA. (A) Protonation of cytosine followed by nucleophilic attack by bisulfite activates the cytosine ring for hydrolytic deamination and b-elimination to produce uracil. (B) A similar process deaminates 5-methylcytosine at a much slower rate than that of cytosine. (C) Protonated bases created at low pH are removed from the DNA chain by glycosyl bond hydrolysis, leading to chain breaks through aldose conversion and b-elimination.

FIG. 2. Scatter in QPCR measurements of DNA methylation in EPS Specimens. The Methylated and Unmethylated promoters at GSTPI is plotted against the sum of the Unmethylated and Methylated APC promoters from different EPS specimens. PCR reaction performance was systematically lower for the GSTPI reaction than for the APC reaction. The linear trend has an R² value of 0.854.

FIG. 3. Receiver Operator Characteristic Curves Comparing PCA3^{DD3}, TMPRSS2:ERG or Sum of Promoter Methylation Levels with Baseline PSA and DRE Results in Predicting Gleason's Sum≧7. ROC data comparing incremental increases in diagnostic performance over baseline covariates Serum PSA+Digital Rectal Exam results. PCA3^{DD3} or TMPRSS2:ERG for the RT-PCR data set: N=74, Gleason's Sum<7=60, Gleason's Sum≧7=14. Performance for Baseline covariates PSA+DRE are plotted in black. Performance for PSA+DRE+PCA3DD3 are plotted in red. Performance for PSA+DRE+TMPRSS2:ERG are plotted in green.

FIG. 4. Receiver Operator Characteristic Curves Comparing PCA3^{DD3}, TMPRSS2:ERG or Sum of Promoter Methylation Levels with Baseline PSA and DRE Results in Predicting Gleason's Sum≧7. ROC data comparing incremental increases in diagnostic performance over baseline covariates Serum PSA+Digital Rectal Exam results. Sum of Promoter Methylation Levels for the bisulfite mediated PCR data set: N=74, Gleason's Sum<7=60, Gleason's Sum≧7=14.

FIG. 5. Receiver Operator Characteristic Curves Comparing PCA3^{DD3} and TMPRSS2:ERG with Baseline PSA and DRE Results in Predicting Biopsy Result. ROC data comparing the incremental increase in diagnostic performance over baseline covariates Serum PSA+Digital Rectal Exam results. PCA3^{DD3} and TMPRSS2:ERG for the RT-PCR data set: N=74, Benign=39, Prostate Cancer=35.

FIG. 6. Recovery of target sequence from bisulfite-treated genomic DNA. High molecular weight DNA was subjected to bisulfite treatment, matrix purification and amplification using the duplex QPCR. Serial dilution of the plasmid standards was used to construct a standard curve for recovery of genomic target DNA from a cell line (HK293) in which the target APC gene is completely unmethylated. Target DNA recovery is plotted as a function of initial DNA concentration receiving bisulfite treatment and matrix purification. That portion of the recovered volume that would represent 200 ng of DNA (assuming 100% recovery at these two steps) was subjected to PCR amplification. A separate PCR reaction was performed using the unconverted primer/probe system to obtain an experimental value for full recovery of the target. Each point is the average of 10 determinations of the ratio of the observed unmethylated copy number to the unconverted copy number Error bars indicate ±1 S.D. (A) Analytical prediction for the recovery based on Equation (4). This graph represents the plot of the equation with the following parameters: θ is a unit-less fraction equal to the ratio of target copies recovered to the total input target copies. Lᵤ=7500 nt, L₁=75 nt, k_{b}=5.2×10³ M⁻¹, ƒ=1/587 nt=0.0017 nt⁻¹. DNA concentration is expressed as the molar concentration of nucleotides ([nt] M) in input genomic single-strands. The points on the graph correspond to 0, 200, 400, 800 and 1600 ng of treated DNA. (B) Analytical prediction for the recovery based on Equation (5). This graph represents the plot of the equation with the following parameters: θ is a unit-less fraction equal to the ratio of target copies recovered to the total input target copies. Lᵤ=7500 nt, Lᵤ=75 nt, k=0.625 h⁻¹M⁻¹, t=16 h, k_{b}=6.00×10³ M⁻¹, DNA concentration is expressed as the molar concentration of nucleotides ([nt] M) in input genomic single-strands. The points on the graph correspond to 0, 200, 400, 800 and 1600 ng of treated DNA.

FIG. 7. Temperature Profiles for targeted regions of the APC, GSTP1, and RARB Promoters. Each profile records the calculated temperature (Ordinate) below which a given region (Abscissa) at or near the PCR target is expected to retain 99% helicity (i.e. Watson Crick Duplex structure). That is to say, given a Watson Crick duplex, less than 1% of the bases at a given sequence position would be melted at any temperature below the indicated temperature.

The calculation was performed using the web based software developed by Tostesen et al., 2005. Each curve is calculated at 75 mM NaCl with an approximate loop entropy factor calculated using the multi-exponential approximation, and the Blossy and Carlon Thermodynamic Parameter (Blossey, 2003). The shaded regions indicate the PCR target sites in each gene, with the position of the forward primer at basepair 500 in the 1000 bp region profiled.

FIG. 8. Receiver Operator Characteristic Curves Comparing PCA3^{DD3}, TMPRSS2:ERG or Sum of Promoter Methylation Levels with Baseline PSA and DRE Results in Predicting Biopsy Result. ROC data comparing incremental increases in diagnostic performance over baseline covariates Serum PSA+Digital Rectal Exam results. A. PCA3^{DD3} or TMPRSS2:ERG for the RT-PCR data set: N=74, Benign=39, Prostate Cancer=35. Performance for Baseline covariates PSA+DRE are plotted in black. Performance for PSA+DRE+PCA3DD3 are plotted in red. Performance for PSA+DRE+TMPRSS2:ERG are plotted in green.B. Sum of Promoter Methylation Levels for the bisulfite mediated PCR data set: N=74, Benign=33, Prostate Cancer=30.

FIG. 9. Primers and probes for TaqMan® QPCR.

FIG. 10. Cloning ideal DNA target standards. Synthetic oligodeoxynucleotides were synthesized so that they corresponded to the deaminated product expected for the methylated or unmethylated sequence. In the unmethylated sequence, each of the cytosines in the genomic sequence was converted to a T in the synthetic DNA. In the methylated sequence, all cytosines except those in CG inucleotides were converted to T. Short oligodeoxynucleotides were annealed and converted to duplex DNAs by primer extension. Blunt-end cloning produced plasmids that carry the target standards. Direct DNA sequencing was used to confirm each sequence.

FIG. 11. A. Overall duplex recoveries APC and GstP1 (60 838 input copies). B. Extension templates for extension and blunt-end cloning.

FIG. 12. Cross reactivity testing. Using the cloned target sequences primer/probe sets were tested for cross reactivity with each target. True target recoveries for cloned standards matched the 100% recoveries expected from the standard curve, while cross target recoveries were negligible.

FIG. 13. Microfluidics separations of the bisulfite-treated DNA. (A) Bisulfite-treated DNA was separated by capillary electrophoresis on microfluidics chips as previously described (Fuller, 2003). Representative results depicted in virtual scan format were replotted to display the profile on a linear molecular length scale. (B) Bisulfite-treated DNA was separated by PAGE using 8M urea to prevent secondary structure formation. Both methods give approximately the same value for the number average molecular weight of single-stranded DNA fragments. Note the differences in abscissas on the two graphs result from the differences between the two methods. The microfluidics system yields molecular lengths calculated from retention times for duplex DNA markers in base pairs. The standard denaturing electrophoresis system is measured in distance from the origin calibrated against RNA markers in nucleotides. The direction of electrophoresis is from left to right in both graphs.

FIG. 14. Gel Electrophoretic Separation of the Components of the APC Amplicon. A. Agarose gel electrophoresis. Synthetic strands corresponding to the sequence of each complementary strand in the 74 bp amplicon were annealed with synthetic strands corresponding to the same complementary strands but containing a 26 nt extension of dT residues at the 3′ end. The four strands (two 74mers and two 100 mers) were annealed in equimolar amounts, and separated by electrophoresis. Marker positions for duplexes are indicated on the right.

Non-Denaturing Polyacrylamide gel electrophoresis. The same four strands were separated by polyacrylamide gel electrophoresis. Marker positions for single strand markers are indicated on the left. Marker positions for duplexes are indicated on the right.

FIG. 15. Gel Electrophoretic Separation of the Components of the RARB Amplicon. The four strands were annealed in the combinations shown and separated by polyacrylamide gel electrophoresis under non-denaturing conditions. Marker positions for single strand markers are indicated on the left.

FIG. 16. Gel Electrophoretic Separation of the Components of the APC Amplicon. The four strands were annealed in the combinations shown, and separated by polyacrylamide gel electrophoresis under non-denaturing conditions. Marker positions for single strand markers are indicated on the left.

FIG. 17. Hypothetical TaqMan® Reaction Scheme. In the first step in the PCR cycle the duplexes are dissociated at high temperature. Thereafter, the reaction mixture is cooled so that the primer (P^{A}) can anneal to single-strand random coils of strand A with a pseudo first order rate constant k₃, and the TaqMan® probe (Tq^{B}) and primer (P^{B}) can anneal to single-strand random coils of strand B with a pseudo first order rate constant k₄. Since the concentration of strand A and strand B is low in the initial phases of the reaction, reassociation to the AB duplex can be neglected and is not shown. However, a rapid unimolecular folding reaction to form single-strand conformers (A_{SSC} and B_{SSC}) is possible and can be expected to serve as a sink that will compete with the production of primer and primer probe complexes. In this model, the concentrations of single-strand random coils of strand A and strand B are rate limiting in the generation of product duplex AB. For the production of fluorescence in the TaqMan® system, only the concentration of strand B random coils is rate limiting.

FIG. 18. The Effect of Single-Strand Conformer Formation on the TaqMan® QPCR. Fluorescence values from the raw QPCR data was plotted directly vs cycle number (plotted Symbols +, ∘, x). The data was fitted to the model developed in analytical equation II using various parameter choices so as to describe the system as one involving the formation of single-strand conformers on each strand (solid lines). For the curves shown, k₁=0.074 sec⁻¹; k₂=20 sec⁻¹; k₃=0.0708 sec⁻¹; k₅=0.88 sec⁻¹; k₆=0.1 sec⁻¹, k₀=8.0×10⁸ [M]⁻¹. Calculated curves (dashed lines) were also plotted with the assumption that SSCs do not form (i.e. constants same as above except, k₁=0 and k₅=0).

FIG. 19. Hypothetical Syber Green Reaction Scheme. In the first step in the PCR cycle the duplexes are dissociated at high temperature. Thereafter, the reaction mixture is cooled so that the primer (P^{A}) can anneal to single-strand random coils of strand A with a pseudo first order rate constant k₃, and the primer (P^{B}) can anneal to single-strand random coils of strand B with a pseudo first order rate constant k₄. Since the concentration of strand A and strand B is low in the initial phases of the reaction, reassociation to the AB duplex can be neglected and is not shown. However, a rapid unimolecular folding reaction to form single-strand conformers (A_{SSC} and B_{SSC}) is possible and can be expected to serve as a sink that will compete with the production of primer and primer probe complexes. In this model, the concentrations of single-strand random coils of strand A and strand B are rate limiting in the generation of product duplex AB. For the production of fluorescence in the Syber Green system, fluorescence measures the concentration of the nascent AB duplexes formed by the extension of both primers P^{A} and P^{B}.

FIG. 20. Bisulfite Conversion. Since the two strands created by bisulfite conversion are not complementary, if the primers lie outside the converted region, then the clonal isolates of bisulfite deaminated DNA can take either of two forms (either C→T or G→A) within a given sequence context. For convenience, these are designated Top-strand isolates or Bottom-strand isolates.

FIG. 21. Bisulfite Sensitivity of Native DNA Isolated from HK 293 Cells. A. The APC Gene Control Region. DNA corresponding to that from the 74 bp amplicon is depicted schematically. Sites in the sequence where cytosines were converted to uracils by bisulfite treatment of native DNA (seen as thymidines in the cloned representatives) are indicated as black squares. Seventeen clones were analyzed, from the larger 318 bp region spanning the 74 bp amplicon studied here. Those with conversions corresponding to the top strand are listed above the sequence in the center of the diagram. Those with conversions corresponding to the bottom strand are listed below the sequence. Positions of the forward and reverse primers along with the position of the probe are indicated. The cluster of conversion indicated by the gray blocks corresponds to the position of a bisulfite-sensitive structure present in the native HK293 DNA on what would be strand B of the amplicon.

B. The RARB Gene Control Region. DNA corresponding to that from the 83 bp amplicon is depicted schematically. Sites in the sequence where cytosines were converted to uracils by bisulfite treatment of native DNA (seen as thymidines in the cloned representatives) are indicated as gray squares. Eleven clones were analyzed, from the 209 bp region spanning the 83 bp amplicon studied here. Those with conversions corresponding to the top strand are listed above the sequence in the center of the diagram. Those with conversions corresponding to the bottom strand would be listed below the sequence except that none were found. Positions of the forward and reverse primers along with the position of the probe are indicated.

### DETAILED DESCRIPTION

The following description of the invention is merely intended to illustrate various embodiments of the invention. As such, the specific modifications discussed are not to be construed as limitations on the scope of the invention. It will be apparent to one skilled in the art that various equivalents, changes, and modifications may be made without departing from the scope of the invention, and it is understood that such equivalent embodiments are to be included herein.

The invention provides markers that have novel utility for the detection of and/or differentiation between prostate cell proliferative disorders and particularly different grades and/or types of prostate cancer. The individual and collective effectiveness of biomarkers such as: hypermethylated GSTPI, APC, RARB and RASSFI; PSA; and TMPRSS2:ERG in the detection and grading of prostate tumors using a non-invasively obtained specimen of prostatic fluid: EPS or Expressed Prostatic Secretion were studied. Each of the markers has diagnostic value in combination with standard serum PSA measurement and digital rectal examination; the combination of serum PSA, DRE and TMPRSS2:ERG expression level is exceptionally effective in predicting biopsy outcome and categorizing Gleason's sum determined at biopsy, when assayed as described herein.

As used herein the term expression shall be taken to mean the transcription and translation of a gene. The level of expression of a gene may be determined by the analysis of any factors associated with or indicative of the level of transcription and translation of a gene including but not limited to methylation analysis, loss of heterozygosity, RNA expression levels and protein expression levels.

Furthermore the activity of the transcribed gene may be affected by genetic variations such as but not limited to genetic mutations (including but not limited to SNPs, point mutations, deletions, insertions, repeat length, rearrangements and other polymorphisms).

The term “CpG island” refers to a contiguous region of genomic DNA that satisfies the criteria of (1) having a frequency of CpG dinucleotides corresponding to an “Observed/Expected Ratio”>0.6, and (2) having a “GC Content”>0.5. CpG islands are typically, but not always, between about 0.2 to about 1 kb, or to about 2 kb in length.

The term “methylation state” or “methylation status” refers to the presence or absence of 5-methylcytosine (“5-mCyt”) at one or a plurality of CpG dinucleotides within a DNA sequence. Methylation states at one or more particular CpG methylation sites (each having two CpG CpG dinucleotide sequences) within a DNA sequence include “unmethylated,” “fully-methylated” and “hemi-methylated.”

The term “AUC” as used herein is an abbreviation for the area under a curve. In particular it refers to the area under a Receiver Operating Characteristic (ROC) curve. The ROC curve is a plot of the true positive rate against the false positive rate for the different possible cutpoints of a diagnostic test. It shows the tradeoff between sensitivity and specificity depending on the selected cutpoint (any increase in sensitivity will be accompanied by a decrease in specificity). The area under an ROC curve (AUC) is a measure for the accuracy of a diagnostic test (the larger the area the better, optimum is 1, a random test would have a ROC curve lying on the diagonal with an area of 0.5; for reference: J. P. Egan. Signal Detection Theory and ROC Analysis, Academic Press, New York, 1975).

The term “hypermethylation” refers to the average methylation state corresponding to an increased presence of 5-mCyt at one or a plurality of CpG dinucleotides within a DNA sequence of a test DNA sample, relative to the amount of 5-mcyt found at corresponding CpG dinucleotides within a normal control DNA sample.

The term “hypomethylation” refers to the average methylation state corresponding to a decreased presence of 5-mCyt at one or a plurality of CpG dinucleotides within a DNA sequence of a test DNA sample, relative to the amount of 5-mcyt found at corresponding CpG dinucleotides within a normal control DNA sample.

The term “bisulfite reagent” refers to a reagent comprising bisulfite, disulfite, hydrogen sulfite or combinations thereof, useful as disclosed herein to distinguish between methylated and unmethylated CpG dinucleotide sequences.

The term “Methylation assay” refers to any assay for determining the methylation state of one or more CpG dinucleotide sequences within a sequence of DNA.

The term “MS.AP-PCR” (Methylation-Sensitive Arbitrarily-Primed Polymerase Chain Reaction) refers to the art-recognized technology that allows for a global scan of the genome using CG-rich primers to focus on the regions most likely to contain CpG dinucleotides, and described by Gonzalgo et al., Cancer Research 57:594-599, 1997.

The term “Methylation assay” refers to any assay for determining the methylation state of one or more CpG dinucleotide sequences within a sequence of DNA.

The present invention provides novel methods and nucleic acids for the detection of and/or differentiation between prostate cell proliferative disorders.

The invention provides a panel of genes, RNA sequences, genomic sequences and/or regulatory regions, the expression levels being indicative of the presence of prostate cell proliferative disorders or features thereof. In particular the methylation status of CpG positions are indicative of the presence of prostate cell proliferative disorders or differentiation between such disorders. Preferred selections and combinations of genes are provided, the methylation analysis of which enable the detection of prostate cell proliferative disorders.

The method includes the diagnosis of prostate cell proliferative disorders; (preferably prostate cancer, prostate carcinoma and/or neoplasms) it is preferred that said genes and/or sequences are selected from the group consisting of: PSA RNA; TMPRSS2:ERG RNA; copy number of GSTPI, APC, RARB or RASSFI DNA; TMPRSS2:ERG Type III or VI fusion RNA; and methylation status of DNA sequences encoding GSTPI, APC, RARB or RASSFI or PCA3.

It is preferred that the prostate cell proliferative disorder is a prostate cancer, prostate carcinoma or prostate neoplasm. In further embodiments the invention provides methods and nucleic acids for the differentiation between non-cancerous types of prostate tissue (including benign prostatic hyperplasia aka “BPH” and normal) from prostate carcinoma.

In other embodiments the invention provides methods and nucleic acids for the differentiation of prostate cancer from normal prostate tissue, tissues originating from other tissues and BPH. In further embodiments the invention provides methods and nucleic acids for the differentiation of prostate cancer from other tissues in a biological sample obtained by a non-invasive means. Furthermore, the invention provides methods and nucleic acids for the identification of biomarkers of prostate cell proliferative disorder in a single non-invasive specimen type. The markers are more effective than those previously known and are effective when used in combination with other diagnostic methods such as Serum PSA and DRE findings.

Preferably the prostate cell proliferative disorder is a prostate cancer, prostate carcinoma or prostate neoplasm. In further embodiments the invention provides methods and nucleic acids for the differentiation between non-cancerous types of prostate tissue (including BPH and normal) from prostate carcinoma. In further embodiments the invention provides methods and nucleic acids for the differentiation of prostate cancer from normal prostate tissue, tissues originating from other tissues and BPH. In certain embodiments, a method of detecting or grading prostate tumors or cancer comprises obtaining one or more expressed prostatic secretion (EPS) samples from a subject and measuring the level of a biomarker EPS.

The present invention provides a method for determining genetic and/or epigenetic parameters of genomic DNA. The method has utility for the improved detection of and/or differentiation between prostate cell proliferative disorders. Although methylation assays for the detection of prostate cancer are known there is currently no molecular classification system for the detection of prostate cell proliferative disorders, nor one that accurately differentiates benign conditions from prostate carcinomas and neoplasms. Further, the present invention is the first to be shown to be effective in detection of aberrant or abnormal prostate cell presence in single non-invasive specimens obtained from previously undiagnosed subjects.

The biosample source may be from any suitable source. Preferably, the source of the sample is selected from the group consisting of cells or cell lines, histological slides, biopsies, paraffin-embedded tissue, bodily fluids, ejaculate, urine, blood, and combinations thereof. Preferably, the source is biopsies, prostatic fluid, bodily fluids, ejaculate, urine, or blood.

Preferably, distinguishing between methylated and non methylated CpG dinucleotide sequences within the target sequence comprises methylation state-dependent conversion or non-conversion of at least one such CpG dinucleotide sequence to the corresponding converted or non-converted dinucleotide sequence within a sequence selected from the group consisting of PSA RNA; TMPRSS2:ERG RNA; copy number of GSTPI, APC, RARB or RASSFI DNA; TMPRSS2:ERG Type III or VI fusion RNA; and methylation status of DNA sequences encoding GSTPI, APC, RARB or RASSFI or PCA3, and contiguous regions thereof corresponding to the target sequence.

Additional embodiments provide a method for the detection of and/or differentiation between prostate cell proliferative disorders, comprising: obtaining a biological sample having subject genomic DNA; extracting the genomic DNA; treating the genomic DNA, or a fragment thereof, with one or more reagents to convert 5-position unmethylated cytosine bases to uracil or to another base that is detectably dissimilar to cytosine in terms of hybridization properties; contacting the treated genomic DNA, or the treated fragment thereof, with an amplification enzyme and at least two primers comprising, in each case a contiguous sequence at least 9 nucleotides in length that is complementary to, or hybridizes under moderately stringent or stringent conditions to a sequence corresponding to a gene encoding a biomarker selected from the group consisting PSA RNA; TMPRSS2:ERG RNA; copy number of GSTPI, APC, RARB or RASSFI DNA; TMPRSS2:ERG Type III or VI fusion RNA; and methylation status of DNA sequences encoding GSTPI, APC, RARB or RASSFI or PCA3, and complements thereof, wherein the treated DNA or the fragment thereof is either amplified, or is not amplified; and determining, based on a presence or absence of, or on a property of said amplified DNA, the methylation state of at least one CpG dinucleotide sequence selected from a sequence encoding a biomarker selected from the group consisting of PSA RNA; TMPRSS2:ERG RNA; copy number of GSTPI, APC, RARB or RASSFI DNA; TMPRSS2:ERG Type III or VI fusion RNA; and methylation status of DNA sequences encoding GSTPI, APC, RARB or RASSFI or PCA3, or an average, or a value reflecting an average methylation state of a plurality of CpG dinucleotide sequences thereof.

Further embodiments provide a method for the detection of and/or differentiation between prostate cell proliferative disorders, comprising: obtaining a biological sample having subject genomic DNA; extracting the genomic DNA; contacting the genomic DNA, or a fragment thereof, comprising one or more sequences encoding a biomarker selected from the group consisting of PSA RNA; TMPRSS2:ERG RNA; copy number of GSTPI, APC, RARB or RASSFI DNA; TMPRSS2:ERG Type III or VI fusion RNA; and methylation status of DNA sequences encoding GSTPI, APC, RARB or RASSFI or PCA3 or a sequence that hybridizes under stringent conditions thereto, with one or more methylation-sensitive restriction enzymes, wherein the genomic DNA is either digested thereby to produce digestion fragments, or is not digested thereby; and determining, based on a presence or absence of, or on property of at least one such fragment, the methylation state of at least one CpG dinucleotide sequence of one or more sequences encoding a biomarker selected from the group consisting of PSA RNA; TMPRSS2:ERG RNA; copy number of GSTPI, APC, RARB or RASSFI DNA; TMPRSS2:ERG Type III or VI fusion RNA; and methylation status of DNA sequences encoding GSTPI, APC, RARB or RASSFI or PCA30r an average, or a value reflecting an average methylation state of a plurality of CpG dinucleotide sequences thereof. The digested or undigested genomic DNA may be amplified prior to said determining.

Additional embodiments provide novel genomic and chemically modified nucleic acid sequences, as well as oligonucleotides and/or PNA-oligomers for analysis of cytosine methylation patterns within sequences encoding a biomarker selected from the group consisting of PSA RNA; TMPRSS2:ERG RNA; copy number of GSTPI, APC, RARB or RASSFI DNA; TMPRSS2:ERG Type III or VI fusion RNA; and methylation status of DNA sequences encoding GSTPI, APC, RARB or RASSFI or PCA3.

In certain embodiments, a method of detecting prostate tumors or cancer and/or categorizing Gleason's Sum of the prostate tumors comprises performing a digital rectal examination on a subject; measuring or detecting the level of PSA in the subject's serum; obtaining one or more expressed prostatic secretion (EPS) samples from the subject; measuring or detecting PSA levels in the EPS; and measuring or detecting a biomarker in the EPS, wherein the biomarker is TMPRSS2:ERG (e.g., TMPRSS2:ERG RNA or TMPRSS2:ERG fusion RNA).

In certain embodiments, a method of detecting prostate tumors or cancer comprises performing a digital rectal examination on a subject, measuring or detecting the level of PSA in the subject's serum and obtaining one or more expressed prostatic secretion (EPS) samples from the subject. The following analyses of the samples are also performed: (a) measuring or detecting PSA levels in the EPS; and (b) measuring or detecting methylated copies of GSTPI, APC, RARB and/or RASSFI by performing PCR, wherein prior to amplifying the nucleic acid (e.g., DNA or RNA), the nucleic acid is treated with bisulfite without being previously denatured. In certain embodiments, the DNA may be previously denatured.

In certain embodiments, a method of detecting prostate tumors or cancer comprises performing a digital rectal examination on a subject; measuring or detecting the level of PSA in the subject's serum; obtaining one or more expressed prostatic secretion (EPS) samples from the subject; measuring or detecting PSA levels in the EPS; and measuring or detecting a biomarker in the EPS, wherein the biomarker is PCA3 (e.g., PCA3 RNA).

A kit for performing any of the above embodiments which includes combinations of the various components and ingredients described herein is also included.

In certain embodiments, a method of detecting prostate tumors or cancer comprises performing a digital rectal examination on a subject, measuring or detecting PSA in the subject's serum; obtaining one or more expressed prostatic secretion (EPS) samples from the subject; measuring or detecting one or more of the markers described above without measuring PSA RNA in the EPS.

The method and nucleic acids according to the invention are used for detection of, screening of populations for, differentiation between, monitoring of, and detection and monitoring of prostate cell proliferative disorders.

The Gleason grading system is based on the glandular pattern of the tumor. Gleason grade takes into account the ability of the tumor to form glands. A pathologist, using relatively low magnification, performs the histologic review necessary for assigning the Gleason grade. The range of grades is 1-5: 1, 2 & 3 are considered to be low to moderate in grade; 4 & 5 are considered to be high grade. When developing this grading system, Gleason noted that the prognosis for a given patient fell somewhere between that predicted by the primary grade and a secondary grade given to the second most prominent glandular pattern. When the two grades were summed, the total Gleason Score was a more accurate predictor of outcome than either of the individual grades. Thus the traditionally reported Gleason score will be the sum of two numbers between 1-5 with a total score from 2-10.

Bisulfite modification of DNA is an art-recognized tool used to assess CpG methylation status. 5-methylcytosine is the most frequent covalent base modification in the DNA of eukaryotic cells. It plays a role, for example, in the regulation of the transcription, in genetic imprinting, and in tumorigenesis. Therefore, the identification of 5-methylcytosine as a component of genetic information is of considerable interest. However, 5-methylcytosine positions cannot be identified by sequencing, because 5-methylcytosine has the same base pairing behavior as cytosine. Moreover, the epigenetic information carried by 5-methylcytosine is lost during, e.g., PCR amplification.

The most frequently used method for analyzing DNA for the presence of 5-methylcytosine is based upon the specific reaction of bisulfite with cytosine whereby, upon subsequent alkaline hydrolysis, cytosine is converted to uracil which corresponds to thymine in its base pairing behavior. However, 5-methylcytosine remains unmodified under these conditions. Consequently, the original DNA is converted in such a manner that methylcytosine, which originally could not be distinguished from cytosine by its hybridization behavior, can now be detected as the only remaining cytosine using standard, art-recognized molecular biological techniques, for example, by amplification and hybridization, or by sequencing. All of these techniques are based on differential base pairing properties, which can now be fully exploited.

The prior art, in terms of sensitivity, is defined by a method comprising enclosing the DNA to be analyzed in an agarose matrix, thereby preventing the diffusion and renaturation of the DNA (bisulfite only reacts with single-stranded DNA), and replacing all precipitation and purification steps with fast dialysis (Olek A, et al., A modified and improved method for bisulfite based cytosine methylation analysis, Nucleic Acids Res. 24:5064-6, 1996). It is thus possible to analyze individual cells for methylation status, illustrating the utility and sensitivity of the method. An overview of art-recognized methods for detecting 5-methylcytosine is provided by Rein, T., et al., Nucleic Acids Res., 26: 2255, 1998.

The bisulfite technique (e.g., Zeschnigk M, et al., Eur J Hum Genet. 5:94-98, 1997), is a well known technique. In all instances, short, specific fragments of a known gene are amplified subsequent to a bisulfite treatment, and either completely sequenced (Olek & Walter, Nat. Genet. 1997 17:275-6, 1997), subjected to one or more primer extension reactions (Gonzalgo & Jones, Nucleic Acids Res., 25:2529-31, 1997; WO 95/00669; U.S. Pat. No. 6,251,594) to analyze individual cytosine positions, or treated by enzymatic digestion (Xiong & Laird, Nucleic Acids Res., 25:2532-4, 1997). Detection by hybridization has also been described in the art (Olek et al., WO 99/28498). Additionally, use of the bisulfite technique for methylation detection with respect to individual genes has been described (Grigg & Clark, Bioessays, 16:431-6, 1994; Zeschnigk M, et al., Hum Mol. Genet., 6:387-95, 1997; Feil R, et al., Nucleic Acids Res., 22:695-, 1994; Martin V, et al., Gene, 157:261-4, 1995; WO 9746705 and WO 9515373).

In one aspect, the present invention provides for the use of any of the known the bisulfite technique, in combination with one or more methylation assays, for determination of the methylation status of CpG dinucleotide sequences within sequences from the group consisting of PSA RNA; TMPRSS2:ERG RNA; copy number of GSTPI, APC, RARB or RASSFI DNA; TMPRSS2:ERG Type III or VI fusion RNA; and methylation status of DNA sequences encoding GSTPI, APC, RARB or RASSFI or PCA3.

Methylation Assay Procedures. Various methylation assay procedures are known in the art, and can be used in conjunction with the present invention. These assays allow for determination of the methylation state of one or a plurality of CpG dinucleotides (e.g., CpG islands) within a DNA sequence. Such assays involve, among other techniques, DNA sequencing of bisulfite-treated DNA, PCR (for sequence-specific amplification), Southern blot analysis, and use of methylation-sensitive restriction enzymes.

For example, genomic sequencing has been simplified for analysis of DNA methylation patterns and 5-methylcytosine distribution by using bisulfite treatment (Frommer et al., Proc. Natl. Acad. Sci. USA 89:1827-1831, 1992). Additionally, restriction enzyme digestion of PCR products amplified from bisulfite-converted DNA is used, e.g., the method described by Sadri & Hornsby (Nucl. Acids Res. 24:5058-5059, 1996), or COBRA (Combined Bisulfite Restriction Analysis) (Xiong & Laird, Nucleic Acids Res. 25:2532-2534, 1997). COBRA. COBRA analysis is a quantitative methylation assay useful for determining DNA methylation levels at specific gene loci in small amounts of genomic DNA (Xiong & Laird, Nucleic Acids Res. 25:2532-2534, 1997). Briefly, restriction enzyme digestion is used to reveal methylation-dependent sequence differences in PCR products of sodium bisulfite-treated DNA. Methylation-dependent sequence differences are first introduced into the genomic DNA by standard bisulfite treatment according to the procedure described by Frommer et al. (Proc. Natl. Acad. Sci. USA 89:1827-1831, 1992). PCR amplification of the bisulfite converted DNA is then performed using primers specific for the CpG islands of interest, followed by restriction endonuclease digestion, gel electrophoresis, and detection using specific, labeled hybridization probes. Methylation levels in the original DNA sample are represented by the relative amounts of digested and undigested PCR product in a linearly quantitative fashion across a wide spectrum of DNA methylation levels. In addition, this technique can be reliably applied to DNA obtained from microdissected paraffin-embedded tissue samples. Typical reagents (e.g., as might be found in a typical COBRA-based kit) for COBRA analysis may include, but are not limited to: PCR primers for specific gene (or bisulfite treated DNA sequence or CpG island); restriction enzyme and appropriate buffer; gene-hybridization oligo; control hybridization oligo; kinase labeling kit for oligo probe; and labeled nucleotides. Additionally, bisulfite conversion reagents may include: DNA denaturation buffer; sulfonation buffer; DNA recovery reagents or kits (e.g., precipitation, ultrafiltration, affinity column); desulfonation buffer; and DNA recovery components.

Expressed Prostatic Secretion (EPS) can be obtained non-invasively by prostatic massage. The performance of Serum PSA, DRE, DNA methylation, PCA3^{DD3} and TMPRESS2:ERG as biomarkers of prostate cancer were evaluated. In one embodiment, EPS were collected in a blinded prospective study from 74 patients undergoing routine biopsy for prostate cancer at City of Hope. Baseline Serum PSA and DRE results were obtained prior to EPS collection by standard methodologies. The EPS specimens were divided into three aliquots. DNA for methylation analysis was prepared from one aliquot, RNA for the production of cDNA was prepared from the second aliquot, and the third aliquot was held in reserve or used for DNA preparation for specimens yielding less than 200 ng total nucleic acid per aliquot. Methylation Sensitive TaqMan® quantitative PCR was employed in the determination of DNA methylation at the APC, RARβ, RASSFI and GSTPI promoter sequences or genes. Also, TaqMan® based reverse transcription PCR was used to determine relative PCA3^{DD3} RNA levels and TMPRSS2:ERG fusion RNA levels was employed. Further, PSA RNA and GADPH RNA levels were also measured in each specimen.

In certain embodiments, DNA methylation analyses were performed on specimens yielding 200 ng or more of total nucleic acid when two of the aliquots were pooled (N=63). Separately the methylation levels at APC, RARB, RASSFI and GSTPI added little to the ROC analyses obtained by measuring serum PSA and performing Digital Rectal Examination (DRE) alone (e.g., AUC=0.630 vs. AUC 0.662-0.705). The sum of all methylation values were greater when coupled to PSA and DRE measurements, (e.g., PSA+DRE+Methyl SUM APC, RARB, RASSFI and GSTPI (AUC=0.721)).

In certain embodiments, RT PCR assays were performed on the same EPS specimens but on a separate aliquot of each specimen. All of the RNA from the aliquot was used to prepare cDNA and the same cDNA preparation was used for each of the RNA based tests (N=74). PCA3^{DD3} expression levels were preferable over PSA and DRE alone comparable to that obtained with the methylation analysis. For example, PCA3^{DD3} levels when coupled with PSA and DRE measurements showed improvement (PSA+DRE+PCA3^{DD3} (AUC=0.692)). TMPRSS2:ERG expression levels when combined with PSA and DRE measurements (PSA+DRE+TMPRSS2:ERG (AUC=0.823)) were more preferable than either DNA methylation or PCA3^{DD3} measurements.

Accordingly, one embodiment of the present invention includes PSA+DRE+TMPRSS:ERG measurements obtained in EPS specimens. This marker also allows differentiation between patients with Gleason's sums greater than 7 and patients with Gleason's sums less than 7 (AUC=0.844).

In another embodiment, GADPH RNA levels are used as a measure of total RNA recovery and PSA RNA levels are a measure of total prostate cell recovery. Excluding patients yielding less than a cutoff value of PSA RNA expression improves the ROC values in each test but reduces the sample size.

The method of analysis may be selected from those known in the art, including those listed herein. Particularly preferred are MethyLight, MSP and the use of blocking oligonucleotides as will be described herein. It is further preferred that any oligonucleotides used in such analysis (including primers, blocking oligonucleotides and detection probes) should be reverse complementary, identical, or hybridize under stringent or highly stringent conditions to an at least 16-base-pair long segment of the base sequences of one or more of the invention biomarkers and sequences complementary thereto. It is further preferred that any oligonucleotides used in such analysis (including primers, blocking oligonucleotides and detection probes) should be reverse complementary, identical, or hybridize under stringent or highly stringent conditions to an at least 16-base-pair long segment of the base sequences of one or more of the invention biomarkers.

The oligonucleotides or oligomers according to the present invention constitute effective tools useful to ascertain genetic and epigenetic parameters of the genomic sequence corresponding to SEQ ID NO: 1 to SEQ ID NO: 76. Preferably, said oligomers comprise at least one CpG, TpG or CpA dinucleotide.

Oligonucleotides or oligomers according to the present invention include those in which the cytosine of the CpG dinucleotide (or of the corresponding converted TpG or CpA dinucleotide) sequences is within the middle third of the oligonucleotide; that is, where the oligonucleotide is, for example, 13 bases in length, the CpG, TpG or CpA dinucleotide is positioned within the fifth to ninth nucleotide from the 5′-end.

The oligonucleotides of the invention can also be modified by chemically linking the oligonucleotide to one or more moieties or conjugates to enhance the activity, stability or detection of the oligonucleotide. Such moieties or conjugates include chromophores, fluorophors, lipids such as cholesterol, cholic acid, thioether, aliphatic chains, phospholipids, polyamines, polyethylene glycol (PEG), palmityl moieties, and others as disclosed in, for example, U.S. Pat. Nos. 5,514,758, 5,565,552, 5,567,810, 5,574,142, 5,585,481, 5,587,371, 5,597,696 and 5,958,773. The probes may also exist in the form of a PNA (peptide nucleic acid) which has particularly preferred pairing properties. Thus, the oligonucleotide may include other appended groups such as peptides, and may include hybridization-triggered cleavage agents (Krol et al., BioTechniques 6:958-976, 1988) or intercalating agents (Zon, Pharm. Res. 5:539-549, 1988). To this end, the oligonucleotide may be conjugated to another molecule, e.g., a chromophore, fluorophor, peptide, hybridization-triggered cross-linking agent, transport agent, hybridization-triggered cleavage agent, etc. The oligonucleotide may also comprise at least one art-recognized modified sugar and/or base moiety, or may comprise a modified backbone or non-natural internucleoside linkage.

It is anticipated that the oligonucleotides may constitute all or part of an “array” or “DNA chip” (i.e., an arrangement of different oligonucleotides and/or PNA-oligomers bound to a solid phase). Such an array of different oligonucleotide- and/or PNA-oligomer sequences can be characterized, for example, in that it is arranged on the solid phase in the form of a rectangular or hexagonal lattice. The solid-phase surface may be composed of silicon, glass, polystyrene, aluminum, steel, iron, copper, nickel, silver, or gold. Nitrocellulose as well as plastics such as nylon, which can exist in the form of pellets or also as resin matrices, may also be used. An overview of the Prior Art in oligomer array manufacturing can be gathered from a special edition of Nature Genetics (Nature Genetics Supplement, Volume 21, January 1999, and from the literature cited therein). Fluorescently labeled probes are often used for the scanning of immobilized DNA arrays. The simple attachment of Cy3 and Cy5 dyes to the 5′-OH of the specific probe are particularly suitable for fluorescence labels. The detection of the fluorescence of the hybridized probes may be carried out, for example, via a confocal microscope. Cy3 and Cy5 dyes, besides many others, are commercially available.

It is particularly preferred that the oligomers according to the invention are utilised for at least one of: detection of; screening of populations for; differentiation between; monitoring of; and detection and monitoring of prostate cell proliferative disorders. This is enabled by use of said sets for the detection of and/or differentiation between prostate cell proliferative disorders in a biological sample isolated from a patient. Particularly preferred are those sets of oligomer that comprise at least two oligonucleotides selected from one of the following sets of oligonucleotides.

It is also anticipated that the oligonucleotides, or particular sequences thereof, may constitute all or part of an “virtual array” wherein the oligonucleotides, or particular sequences thereof, are used, for example, as ‘specifiers’ as part of, or in combination with a diverse population of unique labeled probes to analyze a complex mixture of analytes. Such a method, for example is described in US 2003/0013091 (U.S. Ser. No. 09/898,743, published 16 Jan. 2003). In such methods, enough labels are generated so that each nucleic acid in the complex mixture (i.e., each analyte) can be uniquely bound by a unique label and thus detected (each label is directly counted, resulting in a digital read-out of each molecular species in the mixture).

Preferably, the invention method comprises the following steps: A sample of the tissue or fluid to be analysed is obtained. Preferably, the source of the DNA sample is selected from the group consisting of cells or cell lines, histological slides, biopsies, paraffin-embedded tissue, bodily fluids, prostatic fluid, expressed prostatic secretion, ejaculate, urine, blood, and combinations thereof. Preferably, the source is biopsies, bodily fluids, ejaculate, urine, or blood. The genomic DNA is isolated from the sample. Genomic DNA may be isolated by any means standard in the art, including the use of commercially available kits. Briefly, wherein the DNA of interest is encapsulated in by a cellular membrane the biological sample must be disrupted and lysed by enzymatic, chemical or mechanical means. The DNA solution may then be cleared of proteins and other contaminants e.g. by digestion with proteinase K. The genomic DNA is then recovered from the solution. This may be carried out by means of a variety of methods including salting out, organic extraction or binding of the DNA to a solid phase support. The choice of method will be affected by several factors including time, expense and required quantity of DNA.

The extracted genomic DNA sample is treated in such a manner that cytosine bases which are unmethylated at the 5′-position are converted to uracil, thymine, or another base which is dissimilar to cytosine in terms of hybridization behavior. This will be understood as ‘pretreatment’ or ‘treatment’ herein. The above-described treatment of genomic DNA may be carried out with bisulfite (hydrogen sulfite, disulfite) and subsequent alkaline hydrolysis which results in a conversion of non-methylated cytosine nucleobases to uracil or to another base which is dissimilar to cytosine in terms of base pairing behavior. Fragments of the treated DNA are amplified, using sets of primer oligonucleotides according to the present invention, and an amplification enzyme. The amplification of several DNA segments can be carried out simultaneously in one and the same reaction vessel. Typically, the amplification is carried out using a polymerase chain reaction (PCR). The set of primer oligonucleotides includes at least two oligonucleotides whose sequences are each reverse complementary, identical, or hybridize under stringent or highly stringent conditions to an at least 16-base-pair long segment of the base sequences of one of SEQ ID NOs: 1-76 and sequences complementary thereto.

In an alternate embodiment of the method, the methylation status of preselected CpG positions within the nucleic acid sequences comprising one or more of SEQ ID NO: 1 to SEQ ID NO: 76 may be detected by use of methylation-specific primer oligonucleotides. This technique (MSP) has been described in U.S. Pat. No. 6,265,171 to Herman. The use of methylation status specific primers for the amplification of bisulfite treated DNA allows the differentiation between methylated and unmethylated nucleic acids. MSP primers pairs contain at least one primer which hybridizes to a bisulfite treated CpG dinucleotide. Therefore, the sequence of said primers comprises at least one CpG dinucleotide. MSP primers specific for non-methylated DNA contain a “T” at the position of the C position in the CpG. Preferably, therefore, the base sequence of said primers is required to comprise a sequence having a length of at least 9 nucleotides which hybridizes to a treated nucleic acid sequence according to one of SEQ ID NOs: 1-76 and sequences complementary thereto, wherein the base sequence of said oligomers comprises at least one CpG dinucleotide.

A further embodiment of the method comprises the use of blocker oligonucleotides. The use of such blocker oligonucleotides has been described by Yu et al., BioTechniques 23:714-720, 1997. Blocking probe oligonucleotides are hybridized to the bisulfite treated nucleic acid concurrently with the PCR primers. PCR amplification of the nucleic acid is terminated at the 5′ position of the blocking probe, such that amplification of a nucleic acid is suppressed where the complementary sequence to the blocking probe is present. The probes may be designed to hybridize to the bisulfite treated nucleic acid in a methylation status specific manner. For example, for detection of methylated nucleic acids within a population of unmethylated nucleic acids, suppression of the amplification of nucleic acids which are unmethylated at the position in question would be carried out by the use of blocking probes comprising a ‘CpA’ or ‘TpA’ at the position in question, as opposed to a ‘CpG’ if the suppression of amplification of methylated nucleic acids is desired.

Additionally, polymerase-mediated decomposition of the blocker oligonucleotides should be precluded. Preferably, such preclusion comprises either use of a polymerase lacking 5′-3′ exonuclease activity, or use of modified blocker oligonucleotides having, for example, thioate bridges at the 5′-termini thereof that render the blocker molecule nuclease-resistant. Particular applications may not require such 5′ modifications of the blocker. For example, if the blocker- and primer-binding sites overlap, thereby precluding binding of the primer (e.g., with excess blocker), degradation of the blocker oligonucleotide will be substantially precluded. This is because the polymerase will not extend the primer toward, and through (in the 5′-3′ direction) the blocker—a process that normally results in degradation of the hybridized blocker oligonucleotide. A preferred blocker/PCR embodiment, for purposes of the present invention and as implemented herein, comprises the use of peptide nucleic acid (PNA) oligomers as blocking oligonucleotides. Such PNA blocker oligomers are ideally suited, because they are neither decomposed nor extended by the polymerase.

Preferably, therefore, the base sequence of said blocking oligonucleotides is required to comprise a sequence having a length of at least 9 nucleotides which hybridizes to a treated nucleic acid sequence according to one of SEQ ID NOs: 1-76 and sequences complementary thereto, wherein the base sequence of said oligonucleotides comprises at least one CpG, TpG or CpA dinucleotide.

The fragments obtained by means of the amplification can carry a directly or indirectly detectable label. Preferred are labels in the form of fluorescence labels, radionuclides, or detachable molecule fragments having a typical mass which can be detected in a mass spectrometer. Where said labels are mass labels, it is preferred that the labeled amplificates have a single positive or negative net charge, allowing for better detectability in the mass spectrometer. The detection may be carried out and visualized by means of, e.g. matrix assisted laser desorption/ionization mass spectrometry (MALDI) or using electron spray mass spectrometry (ESI).

Matrix Assisted Laser Desorption/Ionization Mass Spectrometry (MALDI-TOF) is a very efficient development for the analysis of biomolecules (Karas & Hillenkamp, Anal Chem., 60:2299-301, 1988). An analyte is embedded in a light-absorbing matrix. The matrix is evaporated by a short laser pulse thus transporting the analyte molecule into the vapour phase in an unfragmented manner. The analyte is ionized by collisions with matrix molecules. An applied voltage accelerates the ions into a field-free flight tube. Due to their different masses, the ions are accelerated at different rates. Smaller ions reach the detector sooner than bigger ones. MALDI-TOF spectrometry is well suited to the analysis of peptides and proteins. The analysis of nucleic acids is somewhat more difficult (Gut & Beck, Current Innovations and Future Trends, 1:147-57, 1995). The sensitivity with respect to nucleic acid analysis is approximately 100-times less than for peptides, and decreases disproportionally with increasing fragment size. Moreover, for nucleic acids having a multiply negatively charged backbone, the ionization process via the matrix is considerably less efficient. In MALDI-TOF spectrometry, the selection of the matrix plays an eminently important role. For desorption of peptides, several very efficient matrixes have been found which produce a very fine crystallization. There are now several responsive matrixes for DNA, however, the difference in sensitivity between peptides and nucleic acids has not been reduced. This difference in sensitivity can be reduced, however, by chemically modifying the DNA in such a manner that it becomes more similar to a peptide. For example, phosphorothioate nucleic acids, in which the usual phosphates of the backbone are substituted with thiophosphates, can be converted into a charge-neutral DNA using simple alkylation chemistry (Gut & Beck, Nucleic Acids Res. 23: 1367-73, 1995). The coupling of a charge tag to this modified DNA results in an increase in MALDI-TOF sensitivity to the same level as that found for peptides. A further advantage of charge tagging is the increased stability of the analysis against impurities, which makes the detection of unmodified substrates considerably more difficult. More recent methods and devices for detecting DNA hybridization are also suitable for use in the present invention. These are described for example, in U.S. Patent Application Publication No. 2008026854, 20080030737, 20060046306 (the disclosures of which are incorporated by reference in there entirety). The amplified samples obtained during the above described steps of the method are analysed in order to ascertain the methylation status of the CpG dinucleotides prior to the treatment.

In embodiments where the amplificates were obtained by means of MSP amplification, the presence or absence of an amplificate is in itself indicative of the methylation state of the CpG positions covered by the primer, according to the base sequences of said primer. Amplificates obtained by means of both standard and methylation specific PCR may be further analyzed by means of hybridization-based methods such as, but not limited to, array technology and probe based technologies as well as by means of techniques such as sequencing and template directed extension.

In one embodiment of the method, the amplificates synthesized as described above are subsequently hybridized to an array or a set of oligonucleotides and/or PNA probes. In this context, the hybridization takes place in the following manner: the set of probes used during the hybridization is preferably composed of at least 2 oligonucleotides or PNA-oligomers; in the process, the amplificates serve as probes which hybridize to oligonucleotides previously bonded to a solid phase; the non-hybridized fragments are subsequently removed; said oligonucleotides contain at least one base sequence having a length of at least 9 nucleotides which is reverse complementary or identical to a segment of the base sequences specified in the present Sequence Listing; and the segment comprises at least one CpG, TpG or CpA dinucleotide.

In yet a further embodiment of the method, the genomic methylation status of the CpG positions may be ascertained by means of oligonucleotide probes that are hybridized to the bisulfite treated DNA concurrently with the PCR amplification primers (wherein said primers may either be methylation specific or standard).

Another embodiment of this method is the use of fluorescence-based Real Time Quantitative PCR (Heid et al., Genome Res. 6:986-994, 1996; also see U.S. Pat. No. 6,331,393) employing a dual-labeled fluorescent oligonucleotide probe (TaqMan™ PCR, using an ABI Prism 7700 Sequence Detection System, Perkin Elmer Applied Biosystems, Foster City, Calif.). The TaqMan™ PCR reaction employs the use of a nonextendible interrogating oligonucleotide, called a TaqMan™ probe, which, in preferred embodiments, is designed to hybridize to a GpC-rich sequence located between the forward and reverse amplification primers. The TaqMan™ probe-further comprises a fluorescent “reporter moiety” and a “quencher moiety” covalently bound to linker moieties (e.g., phosphoramidites) attached to the nucleotides of the TaqMan™ oligonucleotide. For analysis of methylation within nucleic acids subsequent to bisulfite treatment, it is required that the probe be methylation specific, as described in U.S. Pat. No. 6,331,393, (hereby incorporated by reference in its entirety) also known as the MethylLight™ assay. Variations on the TaqMan™ detection methodology that are also suitable for use with the described invention include the use of dual-probe technology (Lightcycler™) or fluorescent amplification primers (Sunrise™ technology). Both these techniques may be adapted in a manner suitable for use with bisulfite treated DNA, and moreover for methylation analysis within CpG dinucleotides.

A further suitable method for the use of probe oligonucleotides for the assessment of methylation by analysis of bisulfite treated nucleic acids In a further preferred embodiment of the method, the fourth step of the method comprises the use of template-directed oligonucleotide extension, such as MS-SNuPE as described by Gonzalgo & Jones, Nucleic Acids Res. 25:2529-2531, 1997.

In yet a further embodiment of the method, the amplificate is sequenced and subsequent sequence analysis of the amplificate as described by methods known in the art (Sanger F., et al., Proc Natl Acad Sci USA 74:5463-5467, 1977).

Any method known in the art for detecting proteins can be used. Such methods include, but are not limited to immunodiffusion, immunoelectrophoresis, immunochemical methods, binder-ligand assays, immunohistochemical techniques, agglutination and complement assays. (for example see Basic and Clinical Immunology, Sites and Terr, eds., Appleton & Lange, Norwalk, Conn. pp 217-262, 1991 which is incorporated by reference).

Said oligonucleotides may also be present in the form of peptide nucleic acids. The non-hybridized amplificates are then removed. The hybridized amplificates are then detected. In this context, it is preferred that labels attached to the amplificates are identifiable at each position of the solid phase at which an oligonucleotide sequence is located.

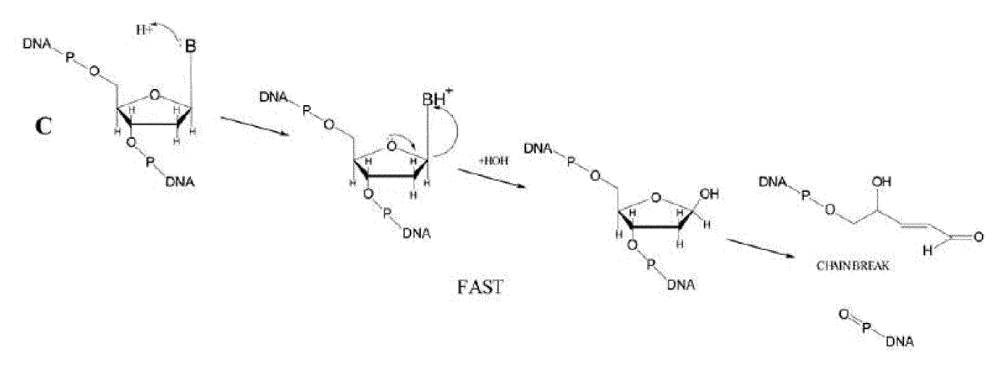

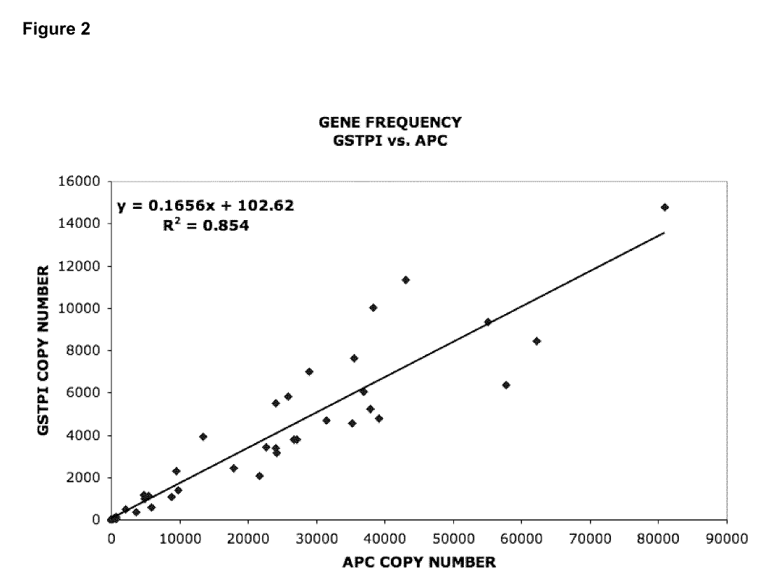

Each of the tests employed herein include use of cycle at threshold (Cₜ) analysis of TaqMan® QPCR data. Linearized plasmid-borne standard sequences were prepared for each test sequence as described in materials and methods below. Assay controls demonstrating specificity and detection limits for the DNA methylation assays were described previously (Munson, 2007) and below. The data in FIG. 2 assess the intrinsic variability in those assays associated with the implementation of the Ct method. Our analyses utilize simple copy numbers obtained from Cₜ analyses related to the plasmid standards.

By using TaqMan® QPCR to quantify each marker one skilled in the art can compare the effectiveness of each marker alone and in combination with other markers in predicting biopsy outcome and Gleason's Sum. Overall, the several methods of analysis reported herein indicate that biomarkers indicative of biopsy outcome are identifiable. For example, any of the three markers provided herein, (the Sum of the Methylated copies of the promoters of GSTPI, APC, RARB and RASSFI, the number of PCA3^{DD3} transcripts or the number of TMPRSS2:ERG fusion transcripts present in EPS specimens) can provide a more effective marker of biopsy outcome than Serum PSA alone in single marker comparisons. These markers are further useful and effective when used in combination with Serum PSA and Digital Rectal Exam findings with the number of TMPRSS2:ERG fusion transcripts providing preferable results based on ROC analysis.

The data provided herein further rank the effectiveness of the markers in a single cohort of previously undiagnosed patients using a single non-invasive specimen type. The TaqMan® (Cₜ) analysis based on the plasmid standards developed herein were used. As a single marker an AUC of 0.600 with a 95% Cl of (0.469, 0.732) compared to the 0.76 with a 95% Cl of (0.64, 0.87) reported previously (van Gils, 2007) was identified. The improvement in AUC over baseline serum PSA analysis was 0.10 (van Gils, 2007)); this is comparable to the improvement over baseline PSA+DRE (0.047) obtained (Table 1). Although the APTIMA® test was applied to EPS (van Gils, 2007), it is also suitable for use in post massage urine samples. For example, where the AUC value for the test is reported to be 0.70 with a 95% confidence interval of (0.58, 0.83). Thus, an improvement in AUC (0.04) over baseline serum PSA (AUC 0.66 with a 95% confidence interval of (0.53, 0.75) as reported previously was measured (van Gils, 2007). This is nearly identical to the improvement obtained in EPS over baseline PSA+DRE obtained with the TaqMan® approach used here: AUC (0.047)

Our analyses rank DNA methylation testing (Sum of Methylated copies) as comparable to PCA3^{DD3} testing. In terms of ease of application, PCA3^{DD3} RNA expression is more cost effective and less time consuming than DNA methylation testing. This is due in part because tumor suppressor gene down regulation requires multiple steps beyond DNA methylation itself that can block the effects of DNA methylation on gene expression at any particular hypermethylated locus. Thus multiple genes should be tested for methylation to obtain results comparable to those obtained with a single RNA expression markers such as PCA3^{DD3}.

The PCR system and plasmid standard developed for the detection of TMPRSS2:ERG fusions comprises a single QPCR analysis; the test may detect two of the known fusions: Type III and Type VI. Of the various fusion types described previously (Wang, 2006), Type III is the most common. Moreover, both Type III and Type VI would have to initiate from the same internal ATG site. Based on the cloning frequencies reported by Wang et al., the single PCR used here should detect 89% of the fusions seen in prostate cancer specimens. Duplication of TMPRSS2:ERG fusions is associated with poor outcome (Attard, 2007) suggesting by analogy with gene amplification during drug resistance that high levels of expression of the fusion are selected during tumor progression. For example Tomlins et al., report that 95% of ERG overexpressing prostate cancers possess an in frame TMPRSS2:ERG fusion. The specificity of the expression of the fusion as a marker for prostate cancer in EPS samples can be a marker of both prostate cancer and aggressiveness.

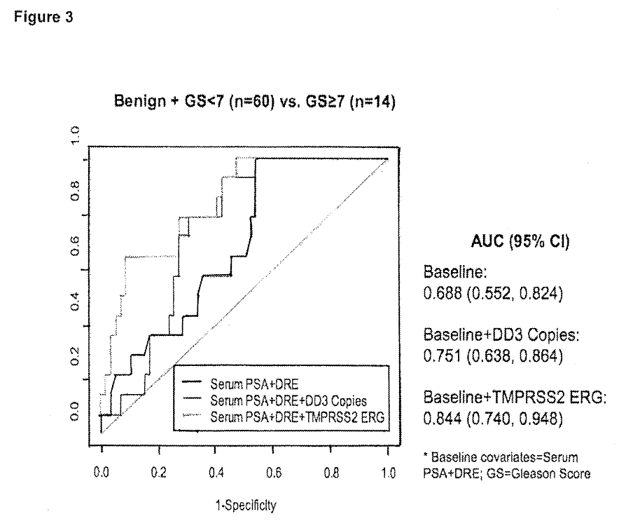

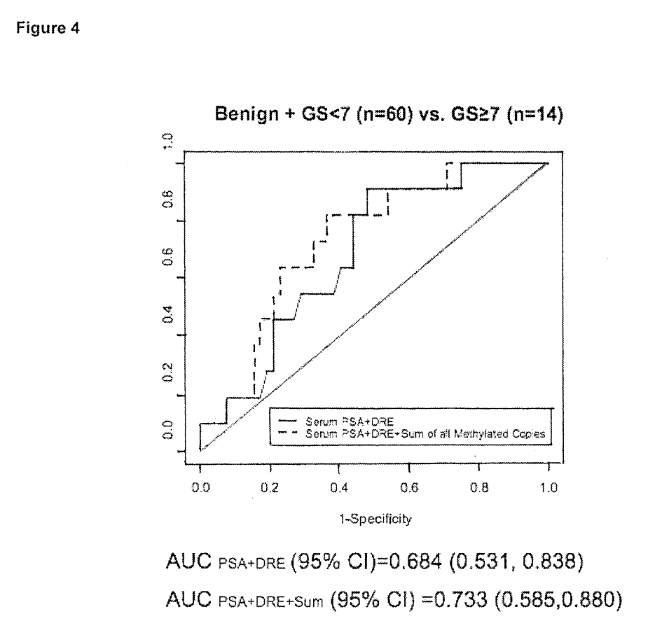

The present invention further confirms the relationship between the presence of TMPRSS2:ERG fusions and prostate cancer aggressiveness. The data provided herein confirms that testing of these biomarkers allows one of skill in the art to distinguish between patients with biopsy Gleason's Sums less than 7 and those with Gleason's sums greater than or equal to 7 (See Table 2 and FIGS. 3, 4). In this application, when coupled with Serum PSA and DRE each test had a particular value. However, TMPRSS2:ERG was the most effective. Its performance in this regard AUC 0.844 and 95% Cl (0.740, 0.948) indicates that it also has utility in Watchful Waiting programs (Active Management of men having PSA levels that have doubled in less than 3 years of PSA velocity greater than 0.75 ng/ml, in addition to a prostate biopsy showing evidence of worsening cancer).

Combining these markers into panels are also of value, e.g. combining Serum PSA, DRE, PCA3^{DD3}, and TMPRSS2:ERG; and PSA, DRE, and TMPRSS2:ERG.

Thus, it is found that each of the three modalities tested here (Sum of Methylated DNA copies in the GSTPI, APC, RARB, RASSFI panel, PCA3^{DD3} RNA expression and TMPRSS2:ERG fusion RNA expression) has diagnostic value in determining biopsy outcome and relative Gleason's Sum and is also of value in that these modalities can be performed on non-invasively obtained EPS specimens. The simple three marker combination panel: Serum PSA, DRE, and TMPRSS2:ERG expression tested as described is one preferred embodiment of the present invention.

Quantitative PCR methods (Herman, 1996; Gonzalgo, 1997) have been introduced that require reference sequences for quantification and as measures of the recovery of intact target DNA. A number of different reference standards have been used in this application. We have used cloned target sequences that reproduce the expected bisulfite-converted target sequence to quantify DNA recoveries in a widely employed TaqMan® quantitative PCR reaction.

Methods of bisulfite treatment employing real-time MS-QPCR were used. Several methods have appeared that avoid the matrix purification step identified as a key difficulty in the recovery of low amounts of DNA. For example, good recovery of low input DNA has been achieved with centrifugal filtration (Boyd, 2004) in place of matrix purification. Moreover, performing the bisulfite treatment in agarose has also been reported to avoid matrix purification and give good recoveries with nested PCR (Olek, 1996). These two approaches may well avoid the losses reported, although they appear not to have been implemented as TaqMan® MS-QPCR analyses. A third approach (Wang, 2006), utilizing nitrocellulose-membrane-bound DNA and hybridization detection of digoxigenin-labeled probes with anti-digoxigenin-AP Fab fragments, obviates not only the matrix purification step but also the PCR. This system is indicated to have desirable qualitative sensitivities.

In general, MS-QPCR reactions are calibrated with in vitro methylated genomic DNA from a cell line or from isolated human lymphocytes (Hoque, 2005). In this calibration method, the mycoplasmal methyltransferase M-SssI is used in excess to completely methylate all CG sites in the genomic DNA methylation standard. Completeness of methylation can be checked with bacterial restriction enzymes. Alternatively, DNA from a cell line known to be completely methylated at the locus of interest can be used as a standard (Toyooka, 2002). Generally, 1 μg of this standard is treated with bisulfite. The recovered product is then serially diluted and amplified to produce the standard curve. Moreover, the methods of the present invention permit the estimation of the level of methylated DNA. Quantification of the amount of unmethylated DNA at the same locus is not often performed in part because a genomic DNA specimen that is completely unmethylated at multiple loci is generally unavailable. Most often, DNA recovery is monitored by amplification of a locus devoid of CG sites. The recovery at this locus (often β-actin or MyoD) is then taken as the denominator in computing a methylation ratio. Here again errors can arise in tumor specimens where inherent changes like DNA amplification or deletion often occur. Moreover, if care is not taken in matching the target lengths of the various genes to that of the recovery locus, different amounts of each target will be degraded during the bisulfite treatment.

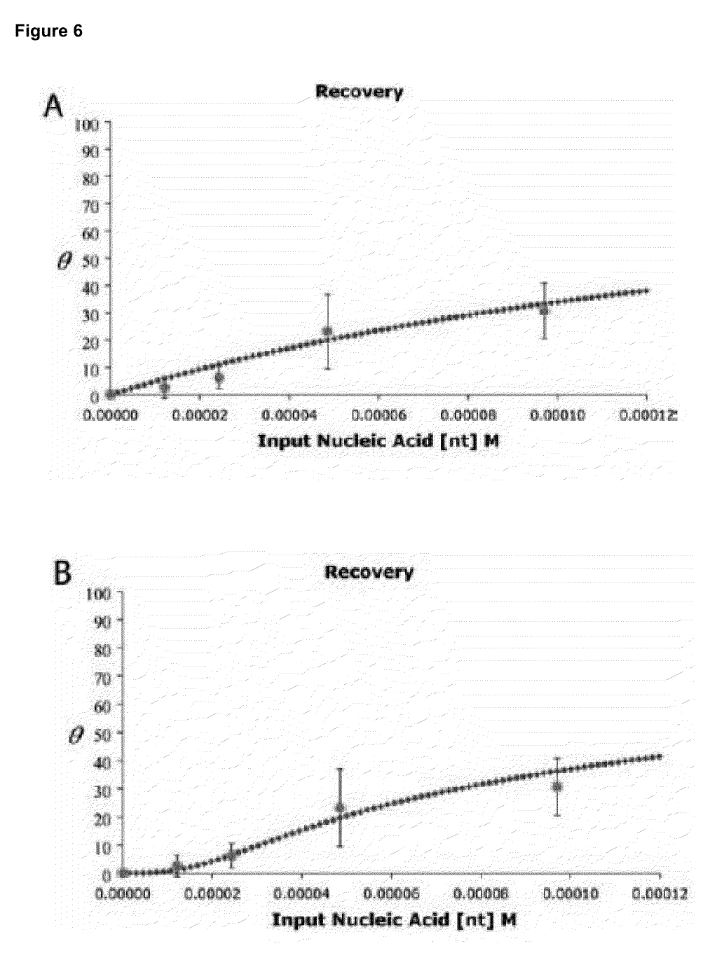

Using the cloned standards and the method described here one is able to compute the ratio of methylated DNA to that of total DNA (methylated & unmethylated DNA) at the locus in question. This method avoids potential artifacts that can occur when the MSssI standard and the specimen DNAs are not treated with bisulfite at the same input concentrations as the specimens (FIG. 6), and provides an internal control for possible amplification, loss of heterozygosity, insertion deletion or repeat expansion at a given locus in genetic diseases and cancer. Preferably, cloned standards as opposed to synthetic duplexes which might also serve as standards are used because plasmid stocks are easily stored and can be easily exchanged between laboratories at almost negligible cost. Thus the use of these cloned standards broadens the scope of the MS-QPCR method and permits it to be more accurately applied.

Finally, in designing MS-QPCR experiments we have found the equation P=e^{-00017L} can be useful in determining the probability P that a target of length L will survive bisulfite treatment, under the conditions described here.

It is further noted that DNA sequences with high G+C content may present concerns in DNA sequencing and PCR amplification because they tend to fold into single-strand conformers (SSCs) during these processes. PCR targets in control regions subject to DNA methylation at the APC, RARB and GSTP1 genes are more than 70% G+C rich and were found to form SSCs during gel electrophoresis. We used bisulfite modification of native DNA to test their effects. Our results show that each of the three PCR targets is rendered accessible to bisulfite without prior denaturation by incubation at 55° C., despite temperature profiling calculations that predict that each of the target sequences should retain more than 99% duplex conformation at any temperature below 85° C. DNA sequencing studies show that the regions of bisulfite accessibility cover each of the amplicons commonly used in DNA methylation analysis with methylation-sensitive QPCR. The data suggests that unusual DNA structures may be present in isolated DNA; secondary structure in standards chosen for quantification may possibly influence measured values. Moreover, the data also show that the innate accessibility of these control regions to the bisulfite reagent permits the analysis of methylation state without prior denaturation of the DNA by sodium hydroxide. Omission of the denaturation step aids in simplifying the MS-QPCR procedure and provides an improvement in recovery of signal at these genes.

In another embodiment, gel electrophoresis was used to demonstrate that the complementary strands of commonly amplified PCR targets from, e.g., the APC, GSTPI and RARB promoters can spontaneously form SSCs. In general, PCR amplification buffers and/or DNA sequencing reagents have been altered so as to suppress the renaturation of duplex DNA (Henke, 1997) or the formation of unusual structures (Dietrick, 1993; Jung, 2002) or both (Musso, 2006). For example, the suppression of unusual secondary and tertiary structure involving G:G, G:G:G:G or C:G:C⁺ bonding are effectively blocked by the use of 7deazaGTP or dITP (Dietrick, 1993; Jung, 2002; Musso, 2006) during amplification since these structures require Hoogsteen pairing of guanine residues. Even so, Watson-Crick pairing is not blocked by these additions, permitting Watson-Crick paired duplex renaturation, along with the formation of Watson-Crick-paired single-strand conformers, and G:C:G:C quadruplex structures like the biloop (Salisbury, 1997).

To better understand the effects of SSC formation on PCR performance a chemical kinetic treatment of the amplification process was developed. The resulting model suggests that single-strand conformers formed at each round of the amplification process generate reaction sinks, and that target availability at the initial round of amplification can be influenced by non-Watson Crick structures that are present in the target DNA when PCR amplification is initiated.

Since this later effect would only be important if an unusual DNA structure were present at the target site in the isolated genomic DNA, we used the method of Raghavan et al. (2006), to test for the presence of unusual DNA structures in the isolated genomic DNA. Our results indicate that such structures may be present in the isolated DNA since PCR targets in control regions subject to DNA methylation at, e.g., the APC, RARB and GSTP1 genes are rendered accessible to bisulfite without prior denaturation by incubation at 55° C., despite temperature profiling calculations that predict that each of the target sequences should retain more than 99% duplex conformation at any temperature below 85° C.

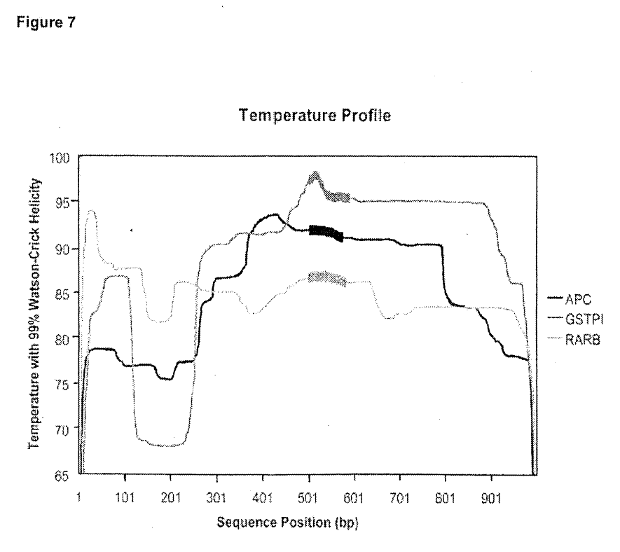

The analytical expression developed herein suggests that the course of fluorescence accumulation in the PCR can be influenced by the formation of SSCs at each round of synthesis, and also by the presence of unusual DNA structures in isolated genomic DNA. The data provided herein indicate that the PCR amplicon in the promoter region of the APC gene is resistant to bisulfite attack at 37° C. However, native DNA from the APC and RARB promoter regions is susceptible to bisulfite attack at 55° C. In general, for sequences like those of the present invention (70% G+C content), incubation at 55° C. would not be expected to promote bisulfite attack unless the DNA sequence contained a non-Watson-Crick structure that can open up significantly at 55° C. Temperature profiling (FIG. 7) supports this contention, since regional helicity of a putative Watson-Crick duplex would be expected to be greater than 99% (Tostesen, 2005) for each of the sequences tested at any temperature below 80° C.

While the potential for unusual DNA structure formation in eukaryotic control regions is of considerable biological significance, commercially it has two important consequences. First, it suggests that the standards used in quantification of PCR results by Cₜ will give reproducible results only if they are prepared in a consistent manner. Data evaluated with genomic DNA, supercoiled, or linearized plasmid DNA as standards will give different quantifications at the same target. Second, it allows one to simplify the chemistry of the MS-QPCR analysis at these sites by omitting the sodium hydroxide denaturation step in an already complicated and low-yield procedure. Our data clearly show that this is the case for the three exemplary amplicons tested herein, and indicate that additional methylation-marked genes will behave similarly.

As noted below, although calculated fluorescence ratios averaged about 1.18, when amplification occurred (Ct<40), there appeared to be a statistically significant (P value<0.05) difference between observed Ct values with and without denaturation (Table 4). Given equation IV below, when Ct analysis is carried out using plasmid standards, one would expect that all parameters except the initial target concentration will be identical. These relatively small differences reflect the level of unusual structure initially present at each site in a given cell line relative to the plasmid standards and/or a difference in recovery after the required desulfonation and purification steps or both.

The relative performance of DNA methylation, PCA3 and TMPRSS2:ERG as biomarkers of prostate cancer in EPS was determined. Methods: EPS was collected in an Institutional Review Board approved, blinded, prospective study from patients undergoing transrectal ultrasound guided biopsy. Serum prostate specific antigen (PSA) and digital rectal examination (DRE) results were obtained by standard methodologies. EPS specimens were divided into three aliquots. DNA methylation levels at the APC, RARβ, RASSF1A and GSTP1 genes were determined by methylation-sensitive quantitative PCR (TaqMan MS-PCR). RNA levels for PCA3 and TMPRSS2:ERG fusions were determined with quantitative expression analyses (TaqMan RT-PCR). DNA methylation analyses were performed only on specimens yielding 200 ng or more of total nucleic acid when two of the aliquots were pooled (N=63). cDNA prepared from a single aliquot was used for each of the RNA-based tests (N=74). Logistic regression was used to analyze the effects of multiple biomarkers in linear combinations. Results: Each biomarker was evaluated for improved performance over baseline PSA and DRE. Methylation levels at APC, RARβ, RASSFI and GSTPI added very little to the receiver operator characteristic analyses over baseline: Area Under Curve (AUC) 0.630 vs. 0.662-0.705. The sum of all methylation values gave a slight improvement over baseline: AUC=0.721. PCA3 expression levels showed an improvement comparable to that obtained with methylation: AUC=0.692, while TMPRSS2:ERG expression levels were significantly more informative than either DNA methylation or PCA3: AUC=0.823. This marker panel was also quite effective in differentiating between patients with Gleason's sums greater than 7 and patients with Gleason's sums less than 7 (AUC=0.844). Conclusions: While each of the biomarker panels tested here has diagnostic value, PSA+DRE+TMPRSS:ERG measurements provide the preferred diagnostic performance in EPS specimens.

In one embodiment, the invention provides a method of detecting and/or differentiation between grades of prostate cancer in a subject. The method comprises: a) determining the expression level of one or more biomarker and b) determining the grade of prostate cancer in the subject according to the level of expression of the one or more biomarker.

In certain embodiments, the predictive power of each biomarker in the study: methylation, PCA3 and TMPRSS2:ERG is improved when more prostate cells are present in the EPS Specimen. f

Wherein the method is for the diagnosis of a prostate cell proliferative disorder, a prostate cancer or to determine the grade of a prostate cancer, the preferred biomarkers are selected from the group consisting of: PSA RNA; ii) TMPRSS2:ERG RNA; iii) copy number of GSTPI, APC, RARB or RASSFI DNA; iv) TMPRSS2:ERG Type III or VI fusion RNA; v) methylation status of DNA sequences encoding GSTPI, APC, RARB or RASSFI or PCA3;

In certain embodiments, kits are provided for performing one or more of the methods described herein. Within the kit, the various components and standards may be divided into separate compartments or in a single, undivided container. In certain embodiments, the kit provides instructions for usage and testing.

A Kit can include for example: a bisulfite-containing reagent; a set of primer oligonucleotides containing at least two oligonucleotides whose sequences in each case correspond, are complementary, or hybridize under stringent or highly stringent conditions to a 16-base long segment of the sequences SEQ ID NO: 1 to SEQ ID NO: 76; oligonucleotides and/or PNA-oligomers; as well as instructions for carrying out and evaluating the described method. In a further preferred embodiment, said kit may further comprise standard reagents for performing a CpG position-specific methylation analysis, wherein said analysis comprises one or more of the following techniques: MS-SNuPE, MSP, MethyLight™, HeavyMethyl™, COBRA, and nucleic acid sequencing.

It is further preferred that a kit comprise a bisulfite-containing reagent; a set of primer oligonucleotides containing at least two oligonucleotides whose sequences in each case correspond, are complementary, or hybridize under stringent or highly stringent conditions to a 16-base long segment of one or more biomarker as described herein, oligonucleotides and/or PNA-oligomers; as well as instructions for carrying out and evaluating the described method. In a further preferred embodiment, said kit may further comprise standard reagents for performing a CpG position-specific methylation analysis, wherein said analysis comprises one or more of the following techniques: MS-SNuPE, MSP, MethyLight™, HeavyMethyl™, COBRA, and nucleic acid sequencing.

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLES

### Example 1

Collection and Nucleic Acid Preparation: EPS was collected from 74 patients. One of the three stored aliquots RNA was converted into cDNA, and used for RT PCR assays on each of the five genes studied on all 74 of specimens. DNA was prepared from the other two aliquots. Of the 74 specimens only 63 specimens yielded total nucleic acid of 200 ng or more, thus only these 63 specimens were used to DNA methylation analysis. QPCR performance was found to be influenced by a number of factors including secondary structure at the target site and in the DNA standard (Clark et al. unpublished). Thus the data obtained are measured relative to the cloned standards described above using Cycle at Threshold analysis (Cₜ) analysis. All data is expressed in copy number determined by comparison to an appropriate standard. Missing data points were assigned zero in the methylation analyses.

1.1 Estimating TaqMan® QPCR Scatter. We noted that methylated copy numbers measured with methylation sensitive QPCR were always at least two orders of magnitude lower than unmethylated copy numbers and could thus be neglected in estimating total gene frequencies from MS QPCR data. Since to the best of our knowledge gene amplification at APC or gene deletion at GSTPI have not been reported in prostate we expected that roughly equal amounts the two genes in each specimen. As can be seen from FIG. 2 the duplex MS QPCR system reports a ratio of about ⅕ for GSTPI/APC. This effect appears to be due to the influence of different amounts of secondary structure in the genomic target, the plasmid standards and the ampl icons of the two genes on QPCR performance (Clark et al. unpublished). Of importance in the present study is the estimate of scatter in QPCR data given by the R² value (0.854) for the linear relationship between the two measured gene copy numbers. This illustration suggests that error propagation in forming ratios will make it unlikely that data will be improved by forming ratios between measurements of different genes in a mixed cell population like that present in EPS.

1.2 Single Biomarker Performance. Since PSA is known to be prostate cell specific and not prostate cancer cell specific it is not a cancer biomarker. This is borne out in ROC analyses where the area under the ROC curve (AUC) is 0.526 for RT-PSA. GADPH is a marker for RNA recovery from all cell types. We collected data on these markers primarily to determine whether or not our RNA preparations contained usable amounts of RNA. We chose not to form ratios with these values as denominators in subsequent analysis in order to avoid the associated error propagation (see Estimating QPCR Scatter above). Many studies (Marks, 2007; van Gils, 2007; Groskopf, 2006) take RT-PSA values as a measure of the proportion of prostate specific cells in the specimen, since it is known to be prostate cell specific. Thus the values can be used as an exclusion criterion for specimens with very low RT-PSA values with the caveats described below.

Single methylation markers GSTPI and APC were only weak biomarkers of biopsy outcome, while RARB and RASSFI appear more informative (Table 5). The sum of the number of methylated copies (Methylation Sum) observed for all four of the genes, when evaluated as a single biomarker, yielded an AUC approaching the average value for each gene evaluated separately. Measured values for the single marker PCA3^{DD3} RNA were effective in predicting biopsy outcome as were measured values for TMPRSS2:ERG fusion RNA. The data in Table 5 suggest that effectiveness of the single biomarkers in predicting biopsy outcome was ordered as follows Methylation Sum≦PCA3^{DD3}<TMPRSS2:ERG based on AUC values.

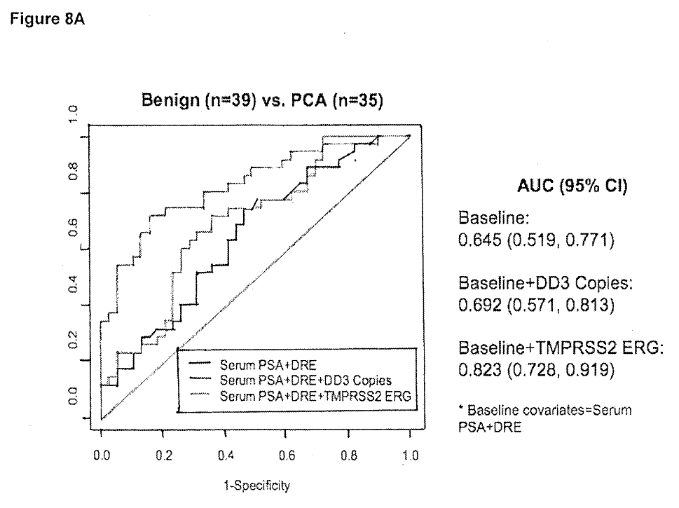

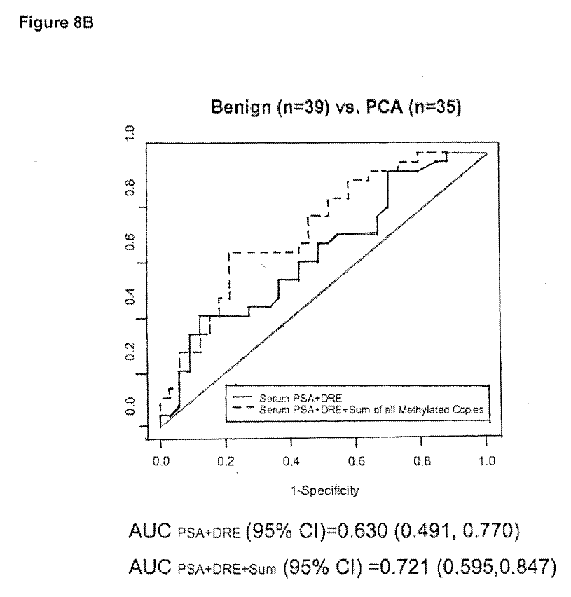

Inclusion of the Serum PSA values and DRE result for each patient improved the effectiveness of the test with each marker (Table 1). Here the effectiveness of the single biomarkers in predicting biopsy outcome (FIG. 8) was ordered as follows PCA3^{DD3}<Methylation Sum<TMPRSS2:ERG based on AUC values.

1.3 Correlation with Gleason's Sum. Given the improved performance exhibited by each marker in predicting biopsy outcome it was of interest to determine whether or not they were also effective in differentiating between high grade and low grade tumors as measured by Gleason's sum at biopsy. The data summarized in Table 1 show that each marker in combination with standard PSA and DRE results added significant value to diagnostic performance. ROC analyses (FIG. 3) show that the methylation markers singly or in combination gave a moderate enhancement in performance based on AUC values. PCA3^{DD3} gave a significant improvement in performance comparable to that seen with the DNA methylation markers. Both DNA methylation and PCA3^{DD3} were significantly less effective than TMPRSS2:ERG in this regard, with the AUC values ordering the tests as follows: Methylation Sum<PCA3^{DD3}<TMPRSS2:ERG.

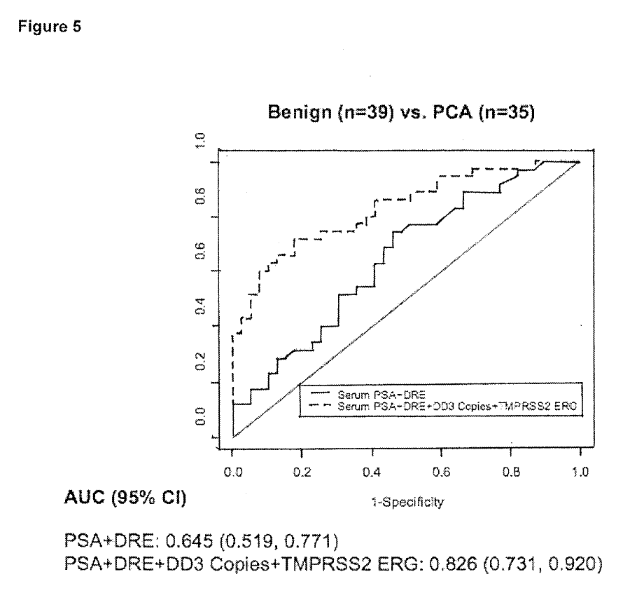

1.4 Combined analysis. The data were also analyzed with various combinations of biomarkers tested simultaneously. FIG. 5 depicts a representative result. In each case TMPRSS2:ERG dominated the diagnostic performance as measured with ROC analysis, with little improvement added by including either PCA3^{DD3} or Methylation Sum. This is readily seen by comparing FIG. 8 with FIG. 5.

### Example 2

### Materials and Methods

2.1 Specimen Collection and Storage. Under an institutional review board approved protocol, men were consented for EPS specimen collection. Prior to biopsy for prostate cancer, a digital rectal examination was performed, followed by prostatic massage and milking of the urethra to collect prostatic secretions. Each specimen was immediately placed on ice and transported to the laboratory where it was suspended in 3 ml of Phosphate Buffered Saline (PBS). The resuspended specimen was dispensed into 1.5 ml screw-capped microcentrifuge tubes in three 1 ml aliquots. The aliquots were sedimented at 8000 g for 5 min. Supernatant fluid was discarded and the tubes containing EPS sediment were stored at −80° C. until use.

### Example 3

Serum PSA Measurement. Serum PSA levels were determined immunometrically with the Vitros Immunodiagnostics Total PSA system (Ortho-Clinical Diagnostics, Rochester, N.Y.).

### Example 4

Digital Rectal Examination (DRE). Current standard of care methods were used in performing DREs. Reported results were analyzed as a dichotomous variable segregating data into DRE: normal or DRE: suspicious for malignancy.

### Example 5

DNA Methylation Detection with Methylation Sensitive TaqMan® QPCR. In certain embodiments, DNA methylation analyses were carried out as previously described in Munson et al., 2007. In Munson et al. 2007 the cell culture was Human kidney 293 cells that were grown as previously described (Shevchuk, 2005). PC3 cells were grown under the same conditions except that the cells were grown in Kaighn's Nutrient Medium F12 (Irvine Scientific, Santa Ana, Calif.) containing 10% Fetal Bovine Serum. PC3 cells were passaged using 1× trypsin-EDTA, at 1:3-1:6.

5.1 DNA isolation. Genomic DNA was isolated using Qiagen's QIAamp® DNA Blood Mini-Kit according to the manufacturer's instructions. The kit-recommended RNAse step was included in order to remove contaminating RNA. The final concentration was determined by spectrophotometry. Qiagen's QIAamp® DNA Blood Mini-Kit was used since it is recommended for purification of DNA from a variety of tissues and bodily fluids as well as cultured cells. For the work described here the cultured-cell protocol in the manual was used.

5.2 Bisulfite treatment. DNA was bisulfite treated using the EZ DNA

Methylation Kit (Zymo Research, Orange, Calif.) according to the manufacturer's instructions. In general, 200-1600 ng of genomic DNA was treated with bisulfite at final concentrations corresponding to 1.33-10.67 ng/μl of genomic DNA. Assuming 100% recovery from the desulfonation and purification steps, that amount of product containing 200 ng of genomic DNA was used for PCR amplification at a concentration of 8 ng/μl.

5.3 Sham-bisulfite treatment. DNA was sham-bisulfite treated by uspending it in the EZ DNA Methylation Kit's bisulfite reagent mixture (Zymo Research, Orange, Calif.) that had been pre-mixed with M-dilution buffer and the matrix-binding buffer so as to prevent the normal hydroxide ion-induced denaturation of the DNA. After a brief mixing it was bound to the purification matrix and eluted from the matrix as described by the manufacturer.

5.4 Gel electrophoretic and microfluidics separation methods. These methods have been described previously (Smith, 1983; Fuller, 2003; Clark, 2003). The DNA 7500 LabChip was found to be most suited to visualization of the molecular length distribution of the bisulfite-treated DNAs. To corroborate estimates of single-strand molecular lengths obtained with nondenaturing microfluidics methodology, separations were also performed on 5% polyacrylamide sequencing gels containing 8M urea (Suzuki, 1994). RNA markers were used to calibrate the polyacrylamide system. The number average molecular weights were determined by use of densitometry measurements on a denaturing polyacrylamide gel using the method described in (Shevchuk, 2005). However an improvement was developed by using Scion Image (Scion Corporation, Frederick, Md.) to calculate the areas under the curve.

5.5 Quantitative PCR. Duplex QPCR reactions used the following cycle profile: 1 hold at 95° C. for 10 min, followed by 50 cycles of: 95° C. for 15 s, 56° C. for 30 s, 72° C. for 30 s. Duplex PCR reactions contained: 0.25 μl Qiagen Hotstar Taq, 2.5 μl 10×Qiagen buffer (providing 1.5 mM MgCl₂), 320 μM dNTPs, 2.0 mM added MgCl₂ (to bring the final MgCl₂ concentration to 3.5 mM) 1.0 μl Q-Solution, 250 nM probe DNA, 900 nM each for forward and reverse primers DNA, 9.95 μl H₂O, 5.0 μl DNA. Uniplex QPCR reactions were the same with the following exceptions: 2.5 mM final MgCl₂, 5 μl Q-solution. The final reaction volume was 25 μl. QPCR conditions for detecting and quantifying the unconverted sequence were identical except that the annealing temperature was 60° C.

Concentrations were determined from a standard curve of the log [input DNA] versus Cₜ determined at a threshold value providing the best efficiency value and linearity in the semilog plot as determined by the Rotor Gene 3000 QPCR analysis software. The plasmid standards have two complementary strands while the genomic DNA targets have two noncomplementary strands once deamination is complete. This means that only one of the two strands is amplified in the bisulfite-mediated PCR. Because of this, the standard curves run for an additional cycle compared to the unknowns. To correct for this the standard curves must be multiplied by a correction factor equal to (1+E)⁻¹, where E is the efficiency of the standard curve.

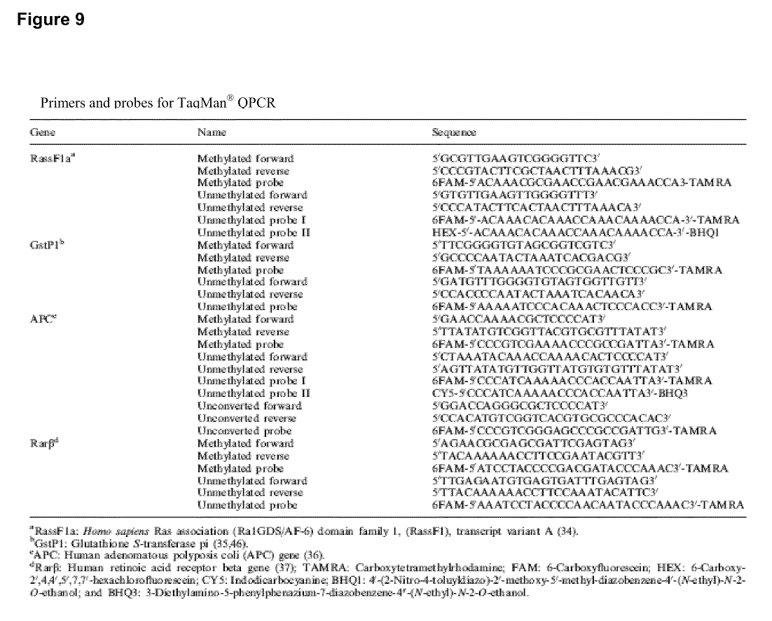

5.6 Synthesis of primers and TAQMAN® probes. Primers (FIG. 9) were purchased from Integrated DNA Technologies (Coralville, Iowa). All Q-PCR probes (FIG. 9) were synthesized in-house on an Expedite® solid-phase DNA/RNA synthesizer on a 1.0 μM scale. The modified phosphoramidites (50-fluorescein, 5′-hexachloro-fluorescein and Cy5), the modified CPG-phosphoramidites (3′-PT-Amino-Modifier C6,3′-BHQ-1,3′-BHQ-3) and TAMRA NHS Ester were purchased from Glen Research (Sterling, Va.). The unmodified phosphoramidite monomers, with either standard or mild protecting groups, along with DNA solid supports and other reagents were purchased from Sigma-Proligo (St. Louis, Mo.) and Applied Biosystems (Foster City, Calif.). The synthesis and deprotection conditions used, were those suggested by Glen Research (Sterling, Va.) for the corresponding reagent. HPLC purification was performed using a PRP-1 column in TeBAA buffer (50 mM tetrabutylammonium acetate buffer, adjusted to pH 7.0 with acetic acid, in a gradient of acetonitrile) or TEAA buffer (50 mM triethylammonium acetate buffer, adjusted to pH 7.0 with acetic acid in a gradient of acetonitrile).

5.7 Synthesis and cloning of ideal standards. Synthetic oligodeoxynucleotides (FIGS. 9, 10) were designed so that they corresponded to the deaminated product expected for the CG-methylated or -unmethylated sequence. In the unmethylated sequence, each of the cytosines in the genomic sequence was converted to a T in the synthetic DNA. In the methylated sequence, all cytosines except those in CG inucleotides were converted to T. Short oligodeoxynucleotides were annealed and converted to duplex DNAs by primer extension. The resulting duplex molecules were treated with T4 Polynucleotide Kinase (NEB, Ipswich, Mass.) and run on a 2% agarose gel. The duplexes were extracted from the gel using a Qiaquick® Gel Extraction Kit (Qiagen, Valencia, Calif.). The plasmid vector, PBluescript II (Stratagene, La Jolla, Calif.), was linearized using R.EcoRV (New England Biolabs, Beverly, Mass.) followed by treatment with Calf Intestinal Alkaline Phosphatase (New England Biolabs, Beverly, Mass.). The plasmid DNA was separated on a 1% agarose gel and the band corresponding to the linearized DNA was gel extracted. Ligation of the duplex fragment and the linear plasmid DNA was carried out overnight at 16° C. using T4 ligase (New England Biolabs, Beverly, Mass.). Blunt-end cloning produced a set of plasmids each carrying an ideal target standard. DNA sequencing was performed at the DNA sequencing facility of the City of Hope Cancer Center to confirm each cloned sequence.

It is important to note here that bisulfite-mediated deamination converts the two target strands so that they are no longer complementary. Thus MS-PCR primers are designed to target only one of the two strands of the target duplex. For this reason, the sequences used in this article correspond only to the target strand utilized in the subsequent QPCR reaction.

5.8 Cloning of unconverted sequences. Unconverted target standard sequences used in the shambisulfite treatment experiments were cloned into PBluescript II as described above. Both sequences were cloned from HK293 genomic DNA. Sequences were confirmed by direct sequencing of the cloned plasmids. The primer set used to clone the unmodified APC fragment for blunt-end cloning were: Forward 5′ACT GCCATCAACTTCCTTGC3′ [SEQ ID NO: 1], Reverse 5′ACCTACCCC ATTTCCGAGTC3′ [SEQ ID NO:2]. The primers and probe sequences used for QPCR reactions were: Forward 5′GGACCAG GGCGCTCCCCAT-3′ [SEQ ID NO:3], and reverse 5′CCACATGTCGG TCACGTGCGCCCACAC3′ [SEQ ID NO:4], Probe 6FAM5′CCCGTC GGGAGCCCGCCGATTG-3′ [SEQ ID NO: 5] TAMRA.

5.9 Cross reactivity experiments For each gene target, primers and probes designed to detect the methylated target were tested in the QPCR reaction to determine whether or not they would amplify the ideal unmethylated standard at a given input copy number and vice versa. QPCR conditions were as given above.

5.10 Search path recovery experiments. In order to increase the search path encountered by the Taq polymerase in binding to an appropriate primer initiation site, increasing amounts of genomic DNA lacking the target sequence (e.g. Micrococcus ysodeikticus DNA which does not contain an amplifiable unmethylated target) were added to the plasmid DNA containing the ideal target sequence. Here, 200 fg of plasmid DNA was used with 200 ng of M. lysodeikticus DNA to provide the same amount of single-copy target that would be present in 200 ng of bisulfite-treated human DNA (i.e. 60 838 copies for a diploid gene).

5.11 Sham-treated genomic DNA. High molecular weight DNA was subjected to shambisulfite treatment for <1 min by adding it to bisulfite reagent pre-mixed with M-dilution buffer and matrixbinding buffer so as to prevent hydroxide-ion-induced denaturation of the DNA. It was then subjected to matrix purification and amplification using the unconverted QPCR primers and probes described above. Since deamination is not expected to occur under these conditions the unmodified plasmid clones described above were diluted appropriately for the construction of the standard curves in these experiments.

5.12 In many cases specimen size is not limiting, thus for many purposes bisulfite treatment of 0.25-4 μg of DNA is recommended, (Shiraishi, 2004; Brena, 2006; Hoque, 2005; Dulaimi, 2004) however, serum and other clinical samples rarely contain this much DNA and quite often bisulfite treatment has been carried out on less than 50 ng of DNA (Bastian, 2005). Given these constraints, multiplex reactions are generally used to conserve specimen. Similar results were obtained throughout this study for uniplex or duplex reactions. Only the results with duplex reactions are reported for simplicity. To study this reaction, we cloned synthetic versions of the desired target sequence (FIG. 10) as recovery standards. These cloned targets are useful in assessing the properties of the reaction in a number of ways.

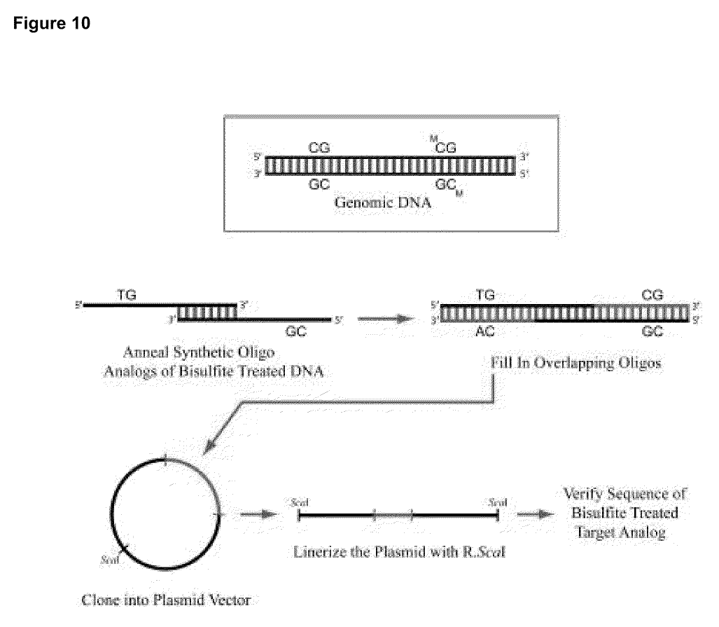

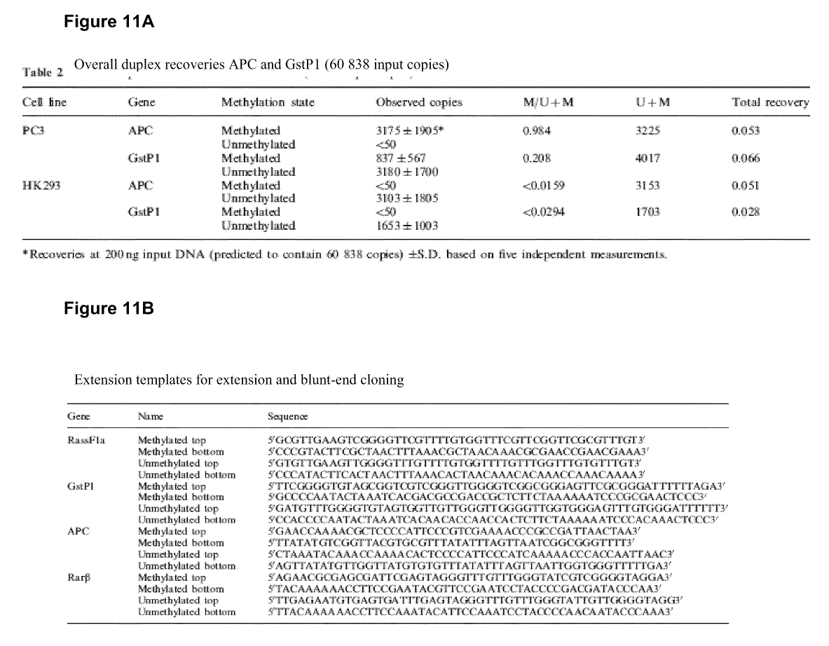

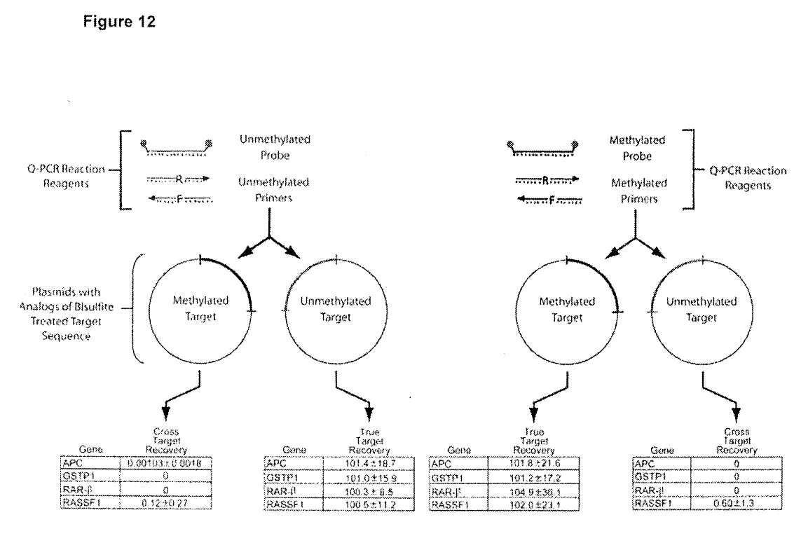

5.13 Cross reactivities. In order to investigate the details of this reaction, it is important to establish that the reactions designed to measure only methylated or unmethylated state of a gene do not cross react. The results of experiments designed to investigate this possibility for each of four commonly used biomarker detection systems (FIG. 11), are depicted in FIG. 12. Here, it is seen that the system is highly selective with cross reactivity accounting for a negligible amount of signal.

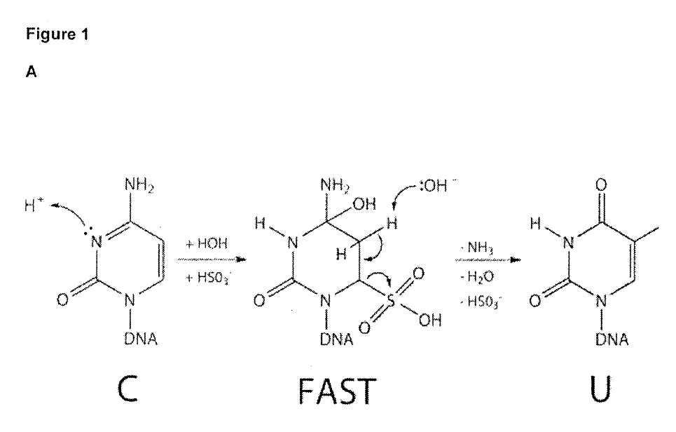

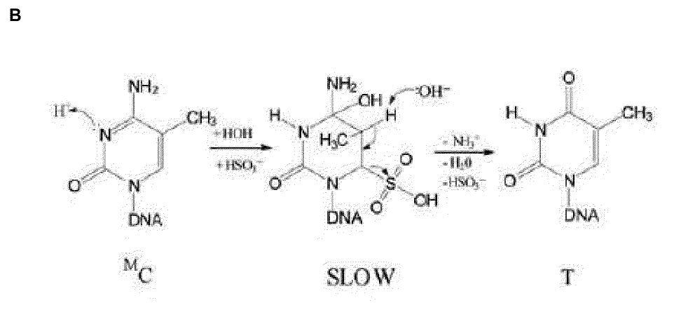

5. 14 Overall recoveries. The existence of the competing reactions depicted in FIG. 1 suggests that significant losses of the desired product can occur, and a priori one might suspect that losses would be a function of input concentration. Thus, we began our experiments by treating 200 ng of DNA with bisulfite. When plasmids containing the desired target sequence (i.e. the sequence expected at the targeted region once complete deamination of the cytosine residues is achieved) were used as copy number standards, we found that recovery was very low and gene-target specific (FIG. 11). That is to say, once the primer sequences were chosen, and primer concentrations and cycle times were optimized for the PCR portion of the reaction, the amount of recoverable input deaminated target sequence was dependent on the cell line used and the gene target. Total recovery for a given gene (i.e. the sum of the copies observed from the methylated (M) and the unmethylated (U) targets) was ˜5% of the input and varied slightly with the gene target used (FIG. 11). Moreover, considerable scatter in the data was observed with input levels at or below 200 ng of genomic DNA. Standard deviations in the observed recovery were on the order of the measurement itself. Recovery in this initial set of experiments was scaled to the expected number of copies present in 200 ng of genomic DNA (60 838 copies for a given single-copy target taken as 100%). This method is open to errors due to inaccuracies in DNA concentration measurement, and subsequent recovery experiments were scaled to the number of copies of the unconverted sequence measured by QPCR.

5.15 Search path recovery experiments. One possible explanation for the low overall recovery of target in these experiments is the relative amount of non-target DNA in the plasmid-borne standards compared to the genomic DNA. In effect, the primers and Taq polymerase can be viewed as being forced to search through considerably more non-target genomic DNA to initiate copying, than they are forced to search through in the standard reactions containing the shorter plasmid target DNA population. Since human DNA contains the target sequence, we used M. lysodeikticus DNA as competitor in experiments designed to detect a decrease of signal associated with DNA seeded with single-copy levels of plasmid DNA target. A 10-12% decrease in signal was detected (data not shown). This finding is not completely unexpected since in most QPCR work this effect is generally offset by the high input concentrations of both Taq polymerase and primers. Clearly this cannot account for the considerable losses we observe.

5.16 Sham-treated DNA. In initial attempts at developing a baseline for recovery estimates we attempted to sham treat the DNA with the bisulfite reagents. Here, DNA was exposed to the bisulfite reagent for as brief a period as possible (generally a maximum of 30 s) before beginning the desulfonation and matrix purification step. As noted by others (Shiraishi, 2004; Grunau, 2001) the conversion can be very rapid. We detected significant amounts of both the converted (i.e. deaminated) and unconverted DNA using the converted and unconverted primer-probe PCR systems for the APC promoter even at short times of exposure, and high input DNA levels (1600 ng). Thus we were unable to use the sham-treated DNA as a baseline for unconverted input levels. Nevertheless, we were able to determine the extent of the reaction at 16 h of exposure to the bisulfite reagent using the unconverted primer probe system for the APC reaction. With the full 16 h of incubation, very little signal could be recovered with this PCR system suggesting that the DNA has been completely converted to the deaminated form by the treatment, whereas the signal from the converted primer probe system was significant. For example, with 1600 ng of genomic DNA (the highest amount used in these experiments), after 16 h of exposure to the bisulfite reagent ˜30% of the input copies were recovered with the converted primer probe system while only ˜2% copies could be detected with the unconverted primer probe system.

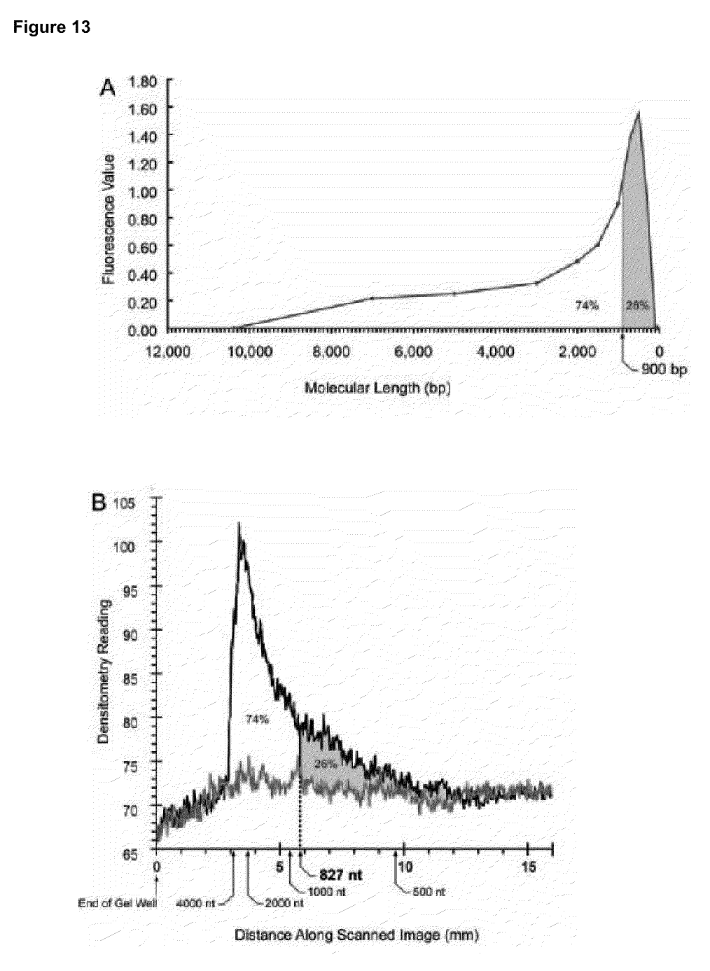

5.17 Measured recoveries of bisulfite-treated DNA. The two competing reactions described above operate to deaminate all cytosine residues while minimizing the breakdown of the DNA. Both reactions are very rapid with complete conversion of all cytosines to uracils in as little as 20 min (Shiraishi, 2004; Wang, 1980) and extensive degradation of the DNA occurring over the same time period. Both deamination and DNA degradation appear to be fast (Shiraishi, 2004; Wang, 1980). To assess the degree of degradation, we determined the size of the bisulfite-treated DNA. Untreated DNA ranged in molecular length from ˜42 000-25 000 bp with a weak smear of smaller DNA fragments that had been sheared during DNA isolation extending to lower molecular lengths, however, bisulfite-treated DNA was extensively degraded. FIG. 13A depicts the observed molecular weight range for the bisulfite-treated DNA as determined by microfluidics-based capillary electrophoresis.

This profile allows us to estimate the probability that single strands from the PCR target will be broken by base loss and subsequent strand-scission (FIG. 1). The distribution of fragment lengths created by random breaks in denatured DNA is given by Equation (1) for a genome of length Λ (Botchan, 1974, Hamer, 1975), where ƒ is the frequency of random breaks, and F_{W}(L) is the weight fraction≧L:

$${F}_{W} ( L ) = \frac{{\int }_{0}^{L} {Lf}^{2} {e}^{- fL} d L}{{\int }_{0}^{Λ} {Lf}^{2} {e}^{- fL} d L}$$

$${F}_{W} ( L ) = 1 - ( 1 + fL ) {e}^{- fL}$$

As previously reported (Shevchuk, 2005), the number average molecular length (L_{N}=1/ƒ) of the distribution of fragments occurs at 26% of the area of the distribution measured from zero molecular weight (Shevchuk, 2005): F_{W}(L_{N})=1−(1+1)e⁻¹=0.26. For the distribution observed after bisulfite treatment, matrix binding and elution (FIG. 13A), L_{N} corresponds to the position of a 900-bp electrophoretic standard. Our experience with the microfluidics separation system is that single-stranded DNA runs ˜25% slower on average than duplex DNA of the same length. Thus the frequency (ƒ) of single-strand breaks is about 1/(L_{N}−0.25L_{N}) or 675 nt if the DNA is completely denatured prior to bisulfite treatment. To confirm this result, we separated the bisulfite-treated DNA under denaturing conditions using 5% polyacrylamide and 8M urea (Suzuki, 1994). As can be seen from FIG. 13B, the estimated number average molecular length using this single-stranded separation system yielding an estimate of ˜587 nt for the number average molecular weight of the bisulfite-treated DNA based on four measurements with a range of 403-827 nt. Given these results, the probability (P) that a single-stranded target sequence of length L will not be broken by bisulfite treatment is given by:

P=(1−ƒ)^{L}≅e^{-ƒL}

For the APC target under study here:

ƒ≅1/587 nt and L=84 nt. Thus P≅0.87

This implies that we should expect only a 13% loss of the APC target simply due to bisulfite-promoted breakdown of the DNA. The calculated expectations for loss due to bisulfite-mediated breakdown do not reflect the experimental results (FIG. 11).

To study this loss in more detail, we used the APC system targeting HK293 genomic DNA. We expect the additional loci studied to behave similarly since recoveries from GstP1 and APC loci were similarly low (FIG. 11). However, the APC system in HK293 cells was chosen for detailed analysis because it is completely unmethylated in the target region as determined by both the direct sequencing of bisulfite-converted clones (Shevchuk, 2005) and the QPCR method described here (FIG. 11). This permits the recovery of unmethylated DNA to be scaled against the experimental QPCR value obtained with the unconverted sequence, thus obviating possible errors in determination of the concentration of the genomic DNA associated with spectrophotometry. As can be seen from FIG. 6, PCR signal recovered at any concentration of bisulfite-treated DNAs was much less than the 87% expected from bisulfite-mediated breakdown frequency measured at higher concentrations. In fact it was dependent on the concentration of DNA present during bisulfite treatment. One might suspect that bisulfitemediated single-strand breaks might somehow be involved in the low recoveries observed in FIG. 6. This would require that the rate of bisulfite-mediated breakdown be actually more extensive at low concentrations of input DNA. However, this actually runs counter to the known properties of the reaction (Shiraishi, 2004). Taken together, these considerations lead one to suspect that size selectivity at the binding and elution step employed in the removal of the bisulfite from the reaction prior to QPCR are responsible for losses experienced in the process.

5.18 Size selection in binding and elution during desulfonation. Assume that there is a lower limit L₁ below which the DNA does not bind to the matrix, and an upper limit Lᵤ above which DNA fragments bind to the matrix but cannot be eluted from it. In this case, the recoverable weight fraction (F_{R}^{C}^{ₜ}) is given by:

F_{R}^{C}^{_{T}}=F_{W}^{C}^{_{T}}(Lᵤ)−F_{W}^{C}^{_{T}}(L₁)

The total concentration of those fragments is:

C_{R}^{C}^{_{T}}=F_{R}^{C}[C_{T}]

C_{R}^{C}^{_{T}}=[F_{W}^{C}(Lᵤ)−F_{W}^{C}(L¹)][C_{T}]

C_{R}^{C}^{_{T}}=[1−(1+ƒLᵤ)e^{ƒL}^{ᵤ}−(1−(1+ƒL₁)e^{-ƒL}^{₁})][C_{T}]

C_{R}^{C}^{_{T}}=[(1+ƒL₁)e^{-ƒL}^{ᵤ}−(1+ƒL₁)e^{-ƒL}^{₁})][C_{T}]

Let θ_{N}=the fraction of intact target DNA recovered after bisulfite treatment, matrix binding and elution. Then the recoverable weight fraction is described by a Langmuir isotherm with a binding constant k_{b}:

$${θ}_{N} = \frac{{k}_{b} {C}_{B}^{{C}_{T}}}{1 + {k}_{b} {C}_{B}^{{C}_{T}}}$$

$${θ}_{N} = \frac{{k}_{b} \{ ( 1 + {fL}_{4} ) {e}^{- {fL}_{4}} - ( 1 + {fL}_{u} ) {e}^{- {fL}_{u}} \} [ {C}_{T} ]}{1 + {k}_{b} \{ ( 1 + {fL}_{4} ) {e}^{- {fL}_{4}} - ( 1 + {fL}_{u} ) {e}^{- {fL}_{u}} \} [ {C}_{T} ]}$$

This relationship provides a reasonably good fit of the data (FIG. 13A) when Lᵤ=7500 nt, L₁=75 nt, and ƒ=1/587 nt=0.0017 nt-1, although there is still a significant deviation from the observed data points at low input concentrations. Apparently the assumption that the cleavage frequency ƒ is independent of DNA concentration over the range tested is not borne out by the data. On the other hand, ƒ can be considered to be a function of input DNA concentration [C_{T}] and time t if all other reaction components are constant (e.g. pH, bisulfite concentration, etc). In this case, dƒ/dt=k[C_{T}] and for any constant time interval t; ƒ=kt[CT]. Substitution in Equation (4) yields:

$${θ}_{N} = \frac{( {k}_{b} \{ \begin{matrix}( 1 + kt [ {C}_{T} ] {L}_{4} ) {e}^{- kt [ {C}_{T} ] {L}_{4}} - \\ ( 1 + kt [ {C}_{T} ] {L}_{u} ) {e}^{- kt [ {C}_{T} ] {L}_{u}}\end{matrix} \} [ {C}_{T} ] )}{( 1 + {k}_{b} \{ \begin{matrix}( 1 + kt [ {C}_{T} ] {L}_{4} ) {e}^{- kt [ {C}_{T} ] {L}_{4}} - \\ ( 1 + kt [ {C}_{T} ] {L}_{u} ) {e}^{- kt [ {C}_{T} ] {L}_{u}}\end{matrix} \} [ {C}_{T} ] )}$$

As can be seen from FIG. 6B this approach gives a much better fit to the C_{T} data. We interpret this to mean that a smaller fraction of the DNA is broken down to the size selection window of the matrix at lower input DNA concentration, compounding the losses at low DNA concentration, and generating the sigmoid nature of the recovery curve in FIG. 6B. To test this possibility, we performed the complete deamination reaction at high DNA concentration (800 ng input DNA) and then put the equivalent of 200 ng through the binding and elution step at the same time that we put the equivalent of 800 ng of the same reaction product through the binding and elution step. In this experiment, the recovery of the target DNA from the 800-ng input specimen was ˜5.8%, or ˜2-fold improvement over the 2.6% recovery observed when 200 ng of DNA is bisulfite treated and subjected to matrix binding and elution. Clearly losses due to the performance of the matrix binding and elution step outweigh those due to single-strand breakdown.

### Example 6

In certain embodiments, DNA methylation analyses were carried out as previously described in Munson et al. 2007 and above in Example 5. Native DNA was treated with bisulfite using the EZ DNA Methylation Kit (Zymo Research, Orange, Calif.) except that the initial sodium hydroxide denaturation step was omitted so as to preserve the secondary structure of the isolated native DNA. Initial exposure of the DNA was to the bisulfite reagent adjusted to pH 5.3. In certain embodiments, data collection and Ct analyses were carried out on the Rotor Gene 3000.

6.1 Reagent Based. Bisulfite conversion of native and denatured DNA was also carried out as described in (Raghavan, 2006), except that 1 μg of DNA was used in the treatments instead of the recommended 5 μg. Briefly, 1 μg of chromosomal DNA was resuspended in 10 ul of TE [pH 7.5]. In a fresh 500 μl tube, 12.5 μl of 20 mM hydroquinone and 457.5 μl of 2.5M sodium bisulfite [pH 5.2 adjusted by adding the require amount of sodium hydroxide] was mixed. For the denaturation step, 1.11 μl of 3M NaOH was added to the DNA and incubated at 37° C. for 15 min. Then 160 μl of the sodium bisulfite-hydroquinone mix was added to the DNA. The reaction was incubated for 16 h at 55 or 37° C. After that, the DNA was purified using Wizard DNA Clean-Up Kit (Promega, Madison, Wis.), according to the manufacturer's instructions. The bisulfite modified DNA was desulfonated with 0.3M NaOH for 15 min at 37° C., and the DNA was recovered by ethanol precipitation.

6.2 Cloning and Sequencing of the Bisulfite-Treated PCR Product. In certain embodiments, the PCR product obtained after bisulfite treatment of native HK293 DNA was amplified with either unconverted APC or unconverted RARB primers. For APC cloning the forward primer was 5′-ACTGCCATCAACTTCCTTGC-3′ [SEQ ID NO: 6] and the reverse was 5′-ACCTACCCCATTTCCGAGTC-3′ [SEQ ID NO: 7]. Amplification consisted of 50 cycles of: 95° C. for 15 s, 56° C. for 30 s, 72° C. for 30 s. For RARB cloning the forward primer was 5′-CAATTCAATCTTTCATTCT-3′ [SEQ ID NO: 8] and reverse 5′-TTGCAAAAAGCCTTCCGAATGCGTTC-3′ [SEQ ID NO: 9] RARB amplification consisted of: 1 cycle of 95° C. for 10 min, 40 cycles of 94° C. for 30 s, 40° C. for 30 s, 72° C. for 30 sec and 1 cycle of 72° C. for 30 min. Each PCR reaction product was then run on a 1% agarose gel and the band corresponding to the 318 bp APC gene product and the 209 bp RARB gene products were gel extracted using the QIAquick Gel Extraction Kit (Qiagen, Valencia, Calif.). 4 μl of extracted DNA was cloned with the pCR 2.1-TOPO Cloning Kit (Invitrogen, Carlsbad, Calif.) according to the manufacturer's instructions. Plasmid clones containing the appropriate sized inserts were sequenced at the City of Hope DNA Sequencing Lab as previously described (Shevchuk, 2005).

6.3 Agarose gel Electrophoresis. Equimolar amounts of each single-stranded oligodeoxynucleotide (10 μM) in several variations were annealed by boiling at 95° C. for 5 min, water bath at 50° C. for 60 min, room temperature for 10 min and then on ice for 10 min. Annealed oligos were run on a 4% MetaPhor® intermediate melting temperature agarose (Lonza, Basel, Switzerland), in TAE (40 mM Tris pH 8, 20 mM acetic acid, 1 mM EDTA) at 4° C. for 3 hours at 50 volts. The gel was stained with 0.5 ug/mL of ethidium bromide.

6.4 Polyacrylamide gel Electrophoresis. A 15% (19:1 Acrylamide:bis-Acrylamide) polyacrylamide sequencing gel was polymerized and annealed oligodeoxynucleotides were run at 1000 volts/50 mAmps for 5.5 hours with the xylene cyanol dye marker at 16 cm from the bottom of the loading well. Single-stranded Oligo-T markers of various lengths, as well as duplex DNA markers were electrophoresed alongside the unknowns. The gels were stained with 0.02% w/v methylene blue and documented. To ensure that all duplex oligodeoxynucleotide bands were visualized with methylene blue, the gel was destained in water, and then stained with 0.5 ug/mL ethidium bromide, and likewise visualized and documented.

6.5 Gel Electrophoretic Analysis of the PCR Product Oligodeoxynucleotides. DNA sequences of high G+C content tend to interfere with PCR amplification. Proposed mechanisms for this interference postulate renaturation of the duplex or unusual structure formation as reaction sinks that decrease empirical efficiency. However, mechanisms that require high concentrations of the product strands (e.g. renaturation of a duplex) can be neglected in developing a model for the data at the low strand concentrations present in the early cycles of the PCR. However, it is important to recall that certain multi-stranded structures like the Hoogsteen-paired G-quadruplex can survive heating to 95° C. and might provide a sink for the reaction that could either decrease the effective target-strand concentration or contribute to the deviation from the empirical model (if present in the isolated genomic DNA). Thus, it is important to determine the stoichiometry of the renatured oligodeoxynucleotides in addition to determining whether or not the individual strands can form single-strand conformers.

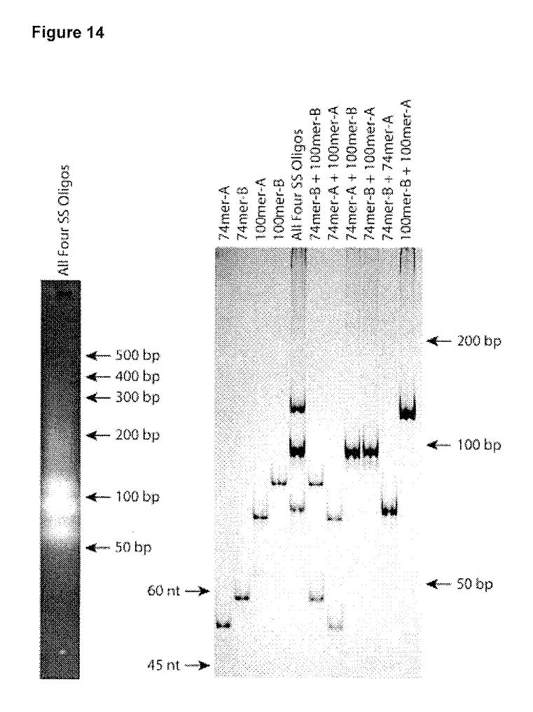

To test these possibilities, we used gel electrophoretic analysis of annealed single-strands of different lengths (Sundquist, 1989; Sen, 1990). In this approach, synthetic 74mer oligodeoxynucleotides corresponding to the PCR amplicon were prepared with or without a 26 nt T-extension. The T-extension is intended to increase the electrophoretic mobility of the annealed product. For a two-stranded structure, three distinct electrophoretic forms are expected when all four strands are annealed, for a three-stranded structure, 4 electrophoretic forms are expected, and for a four-stranded structure five electrophoretic forms should be observed. The method does not distinguish between an A or B form duplex and a bi-loop (Salisbury, 1997) however, since each of these structures require two strands in equimolar quantities. The results for the APC amplicon are shown in FIG. 14. When all four strands are annealed, both the agarose gel electrophoretic separation (FIG. 14) and the native polyacrylamide gel separation (FIG. 14) resolve three bands in roughly the 1:2:1 intensity pattern expected for a duplex or a biloop. In either case, the primary form is composed of two strands. The presence of single-strand conformers of both strands under these conditions is demonstrated in this gel since each 74mer runs well ahead of the 60mer oligo dT marker in the gel. Moreover, the lack of secondary bands when the 74mers are annealed in the presence of the 74mer of the same sequence with a 26 nt dT extension rules out the formation of multi-stranded structures formed between identical sequences.

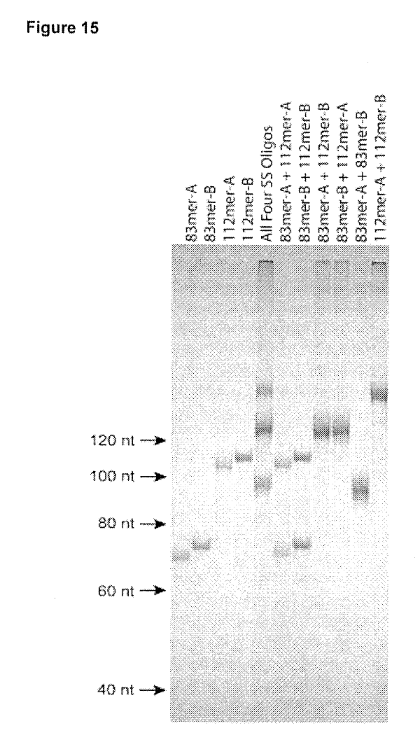

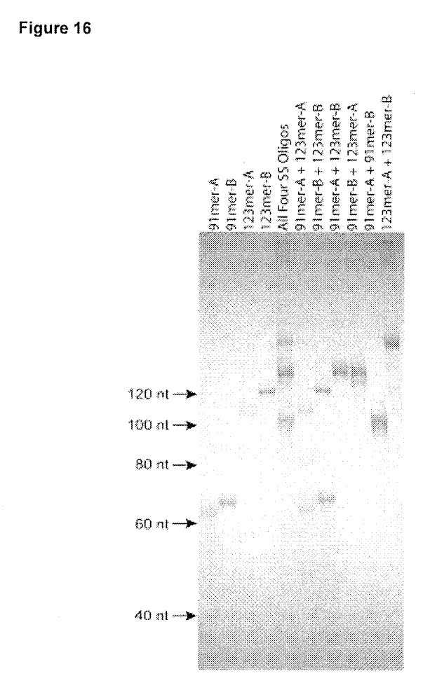

Two other amplicons studied here, that from the RARB control region (FIG. 15) and that from the GSTP1 control region (FIG. 16) gave similar results.

6.6 QPCR Performance at a Site of Unusual DNA Structure Formation. Given that each of the amplicons studied here can form SSCs, it becomes important to develop a kinetic approach to the PCR that overtly includes this phenomenon. To develop this analysis it is important to first study the assumptions of the empirical approach most often employed in these analyses.

6.7 Empirical Approach. Neither the TaqMan® nor the intercalating dye (e.g. Syber Green) approach can follow the full course of the amplification process because the fluorescent signal in early cycles is masked by the background fluorescence produced from the quenched probe or the unintercalated chromophore. In general however, the first measured cycles have been considered to be in an exponentially increasing phase of the process that is described by the empirical relationship:

F(C_{N})−F_{bkd}=(F₀−F_{bkd})(1+E)^{C}^{_{N}}

or for baseline corrected, often referred to as normalized, data:

F(C^{N})=F₀(1+E)^{C}^{_{N}} I

Where E is an empirical constant called the efficiency of the reaction, F is the fluorescence observed at the end of each cycle, F₀ is the fluorescence corresponding to that of the initial target sequence concentration, F_{bkd} is the fluorescence produced by the quenched probe and C_{N} is the cycle number. In this method (Ramakers, 2003) a log transformation of the data permits the determination of F₀ by extrapolation of the log-linear region of the data to cycle C₀ for Syber Green monitored QPCR or C₁ for TaqMan® monitored QPCR. In practice, however, this extrapolation using the log-linear method (Ramakers, 2003) is rarely used, quite possibly because the difficulty in choosing the best log-linear region of the data can result in variations in the extrapolated F₀ value, even with highly reproducible data.

When single-strand conformers form, the empirical equation takes the form:

F(C_{N})=F₀^{S}(1+E_{S})^{C}^{_{N}}

where F₀^{S} is the effective concentration of target DNA at the start of the reaction and E_{S} is the efficiency of the reaction when SSC's reform at each cycle. The value of E_{S} depends on the nature of the equilibrium between random coils accessible to primers, and the SSC's that are inaccessible to primers and probe. An analytical approach that obtains k_{S} and E_{S} in terms of the random coil/SSC equilibria can be developed for the two systems as follows.

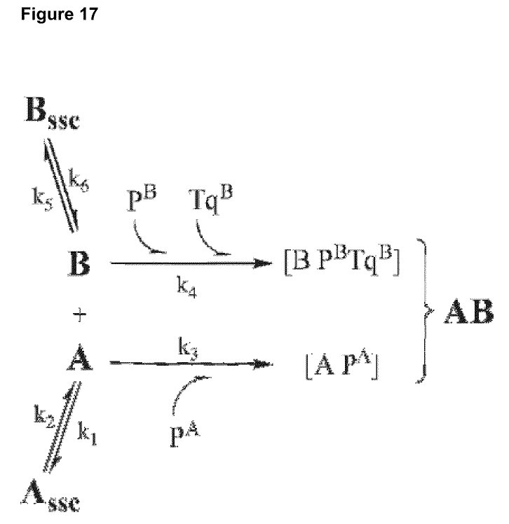

6.8 Analytical Approach to the QPCR Process Monitored by TaqMan® Probes. In order to model this PCR process we assume that the vast molar excess of primer, probe, dNTPs and the high affinity of the Taq DNA polymerase make the kinetics of each cycle in the PCR dependent only on the concentration of the target-strand random coils. This is equivalent to the chemical kinetic assumption that the rate of development of fluorescence due to Taq polymerase degradation of the probe is pseudo first order in the concentration of the single-strand random coil complementary to the forward primer and the probe present at the beginning of the cycle (FIG. 17). We make the distinction between single-strands and single-strand random coils since a single-strand conformer like a DNA hairpin or other folded structure made up of a single-strand should occlude the primer and/or probe binding sequence and slow the PCR reaction by acting as a reaction sink. With these assumptions, we can obtain an analytical expression for the accumulation of fluorescence during the reaction (see Example 10).

An important distinction between the reaction monitored by Syber Green (see below) and the reaction monitored by the TaqMan® probe method is that chromophores released at each cycle fluoresce during subsequent cycles, the accumulated fluorescence F_{A}(C_{N}) is given by:

$${F}_{A} ( {C}_{N} ) = {\int }_{1}^{{C}_{N}} {{k}_{0} ( \frac{{k}_{6}}{{k}_{5} + {k}_{6}} ) [ AB ]}_{0} {e}^{{C}_{N} {k}_{3} {k}_{Tq} t} d {C}_{N}$$

$${F}_{A} ( {C}_{N} ) = \frac{{k}_{0}}{{k}_{3} {k}_{Tq} t} {( \frac{{k}_{6}}{{k}_{5} + {k}_{6}} ) [ AB ]}_{0} {e}^{{C}_{N} {k}_{3} {k}_{Tq} t} {\vert}_{1}^{{C}_{N}}$$

Since the amount of flourescence at the first cycle is undetectable, it can be neglected and we have:

$${F}_{A} ( {C}_{N} ) = \frac{{k}_{0}}{{k}_{3} {k}_{Tq} t} {( \frac{{k}_{6}}{{k}_{5} + {k}_{6}} ) [ AB ]}_{0} {e}^{{C}_{N} {k}_{3} {k}_{Tq} t} \qquad II$$

It is important to note that when:

$${F}_{0} = \frac{{k}_{0}}{{k}_{3} {k}_{Tq} t} {( \frac{{k}_{6}}{{k}_{5} + {k}_{6}} ) [ AB ]}_{0}$$

$$and$$

$${k}_{3} {k}_{Tq} t = ln ( 1 + {E}_{S} )$$

Equation II reduces to the form of the more familiar empirical equation:

F(C_{N})=F₀^{S}(1+E_{S})^{C}^{_{N.}}

Moreover when SSCs do not form at a given target sequence, k₁ and k₅ can be taken as zero and equation II reduces to:

$${F}_{A} ( {C}_{N} ) = {\frac{{k}_{0}}{{k}_{3} t} [ AB ]}_{0} {e}^{{C}_{N} {k}_{3} t}$$

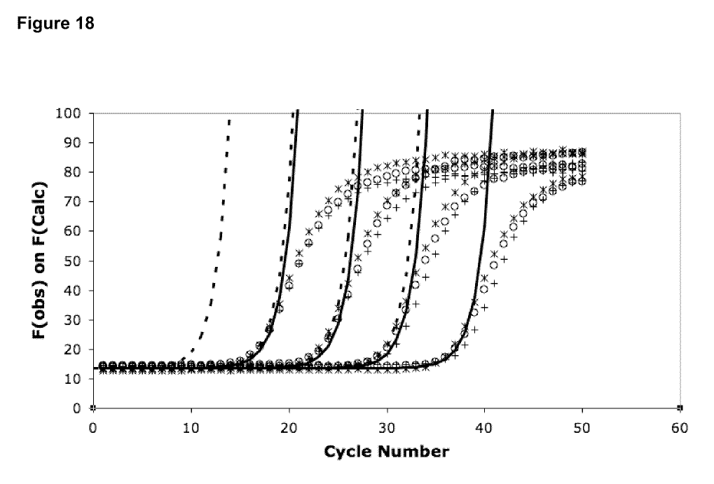

A good fit of the data is obtained from the model when both strands are assumed to form single-strand conformers (FIG. 18).

Real-time PCR assumes that the accumulation of the fluorescence during the amplification process is proportional to the accumulation of the amplicon (Peirson, 2003). As noted above, this accumulation of fluorescence at cycle C_{N} is most often described with the empirical relationship given in equation I. Since the ability of the chromophore to fluoresce is stable, fluorescence accumulates as free chromophore at each cycle in the PCR in the TaqMan® QPCR. By assuming that the course of the reaction between points of measurement is continuous and is described by equation I, F₀ can be obtained directly by extrapolation. In this method (Ramakers, 2003) a log transformation of the data permits the determination of F₀ by extrapolation using the log-linear method (Ramakers, 2003) as noted above.

In practice, however, the ᵢC_{T} or cycle-at-threshold approach is more often employed. Here, one chooses a threshold value (F_{T}), often in the log-linear region of each of several curves produced by dilution of a standard concentration of target DNA where the fluorescence at the threshold value is a constant. Treating data as unitless so that logarithms can be taken makes the C_{T} analysis independent of the choice of method used to measure the input target. It can be given in any intensive measure (e.g. molarity, copies/reaction) as long as the unit chosen is consistently used.

For baseline corrected data:

log(ᵢF₀)=−ᵢC_{T} log(1+E)+log(F_{T})

Moreover, if one assumes that F=k₀[AB] where [AB] is the molar concentration of the chromophore released per mole of duplex AB produced, then:

log(k₀[AB]₀ⁱ)=−C_{T}ⁱ log(1+E)+log(k₀[AB]_{T})

Most systems simply drop the proportionality constant k₀ and use the log of the concentration at threshold: log([AB]_{T}). The curve-fitting algorithm seeks to optimize the linearity (i.e. maximize the R² value) for the semilog plot of log([AB]₀ⁱ)_{VS} C_{T}ⁱ by adjusting the choice of log([AB]_{T}). Log₁₀ is most often used in the analysis but the natural log could also be used to express the familiar relationship:

ln(k₀[AB]₀ⁱ)=−C_{T}ⁱ ln(1+E)+ln(k₀[AB]_{T})

This method is also valid at a target prone to unusual structure formation. However, when equation II is used to describe the system, one has:

$$ln ( \frac{{k}_{0}}{{k}_{3} {k}_{Tq} t} {( \frac{{k}_{6}}{{k}_{5} + {k}_{6}} ) [ AB ]}_{0}^{i} ) = - {C}_{T}^{i} {k}_{3} {k}_{Tq} t + ln ( {{k}_{0} [ AB ]}_{T} )$$

and points on the abscissa of the semi log plot in the C_{T} analysis will now equal

$$ln ( \frac{{k}_{0}}{{k}_{3} {k}_{Tq} t} {( \frac{{k}_{6}}{{k}_{5} + {k}_{6}} ) [ AB ]}_{0}^{i} )$$

and the slope of the plot will be k₃k₃k_{Tq}t instead of ln(1+E). However, the validity of the standard curve used in C_{T} analysis will depend on the extent to which the standard used actually reflects the structural state of the isolated genomic DNA at the first cycle in the PCR. This is because it is reasonable to assume that the level of folding during amplification will be identical for both the standard and the target. If the standard has uniform Watson-Crick structure while a fraction of the isolated DNA retains an unusual structure involving the target region, then the amount of target will be underestimated if the unusual structure cannot be amplified. Conversely, if the DNA standard possesses a non-amplifiable structure involving the target region, while the genomic DNA has a uniform Watson-Crick structure, then the amount of target will be overestimated.

6.9. A related phenomenon, strand bias in the analysis of DNA methylation data (Shen, 2007; Wojdacz, 2007), can be easily understood in terms of this model as a difference in the tendency of the two strands to form single-strand conformers. Suppressing strand bias (Shen, 2007; Wojdacz, 2007) would require choosing primer sites and reaction conditions that make equilibria defined by the values of k₅/k₆ and the values of k₁/k₂ approximately equal during the amplification cycles (FIG. 17). Even so, certain structures, like the G quadruplex at the human Myc gene promoter, might survive bisulfite treatment and adversely affect the PCR on the G-rich strand in that region.

Given the known tendency of G+C rich sequences to form single-strand conformers and the high Tm values observed for such sequences, assuming that single-strand conformers can occur at subsequent cycles in both the target and the standard is a reasonable first approach. The data provided in FIG. 18 shows that the expression in equation II provides a good fit for the behavior of TaqMan® data during early cycles when isolated plasmid standards corresponding to the APC promoter target are employed. In this representation, choosing finite values for k₁, k₂, k₅, and k₆ yields a good fit to the data. The ideal curve (i.e. the curve generated when SSC formation is neglected by setting both k₁ and k₅ equal to zero) emerges earlier in each case.

Two features of the invention are of note. First, for the TaqMan® system, the growth of the product between measured data points is exponential (See Example 10), thus the assumption of continuity for the purpose of interpolating the data between recorded points is not rigorously met. Second, when a single-strand conformer is formed on the strand that serves as template for the production of the strand that binds the probe, the concentration of the available template is reduced below the actual amount present, greatly slowing the amplification process.

6.10 Analytical Approach to the QPCR Process Monitored by an Interating Dye. It is useful to compare the analysis developed for the TaqMan® QPCR to that for Syber Green QPCR, since the two methods are quite distinct in the manner by which fluorescence arises. As is readily apparent from the following, the TaqMan® QPCR equations do not describe the Syber Green® QPCR process accurately.

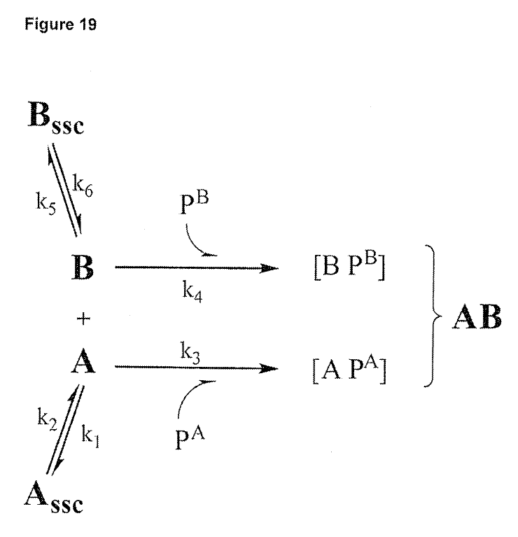

In the Syber Green® approach, the dye must intercalate into base-paired DNA after each extension step. Thus we are interested in the rate of production of each strand in the reaction system given in FIG. 19 because both duplex DNA and SSCs must be considered capable of binding the intercalating dye and producing fluorescence.

$$\frac{d [ AB ]}{d t} = {k}_{3} [ A ] + {k}_{4} [ B ]$$

Using the same reasoning employed in the TaqMan® analysis (see Example 10) we have:

$$\frac{d [ AB ]}{d t} = [ {k}_{3} ( \frac{{k}_{2}}{{k}_{1} + {k}_{2}} ) + {k}_{4} ( \frac{{k}_{6}}{{k}_{5} + {k}_{6}} ) ] [ AB ]$$

and for background corrected data:

$${F}_{A} ( {C}_{N} ) = {k}_{0} {{k}_{Sy} [ AB ]}_{0} {e}^{{C}_{N} {k}_{Sy} t} \\ Where \\ {k}_{Sy} = {k}_{3} ( \frac{{k}_{2}}{{k}_{1} + {k}_{2}} ) + {k}_{4} ( \frac{{k}_{6}}{{k}_{5} + {k}_{6}} ) \qquad III$$

In this case the equation is much simpler, since fluorescence occurs only when Syber Green intercalates into the base paired DNA. Unlike the TaqMan® procedure, this measures the total amount of base pairs produced at each cycle, and integration over previous cycles is not required. On the other hand the absolute yield of fluorescence is generally lower with Syber Green because fluorescence does not accumulate.

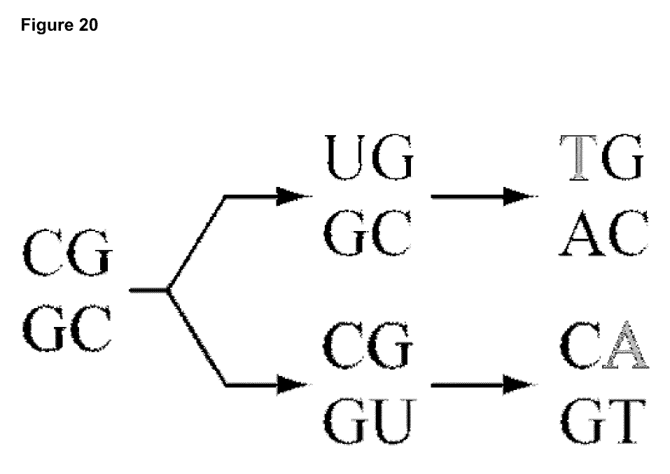

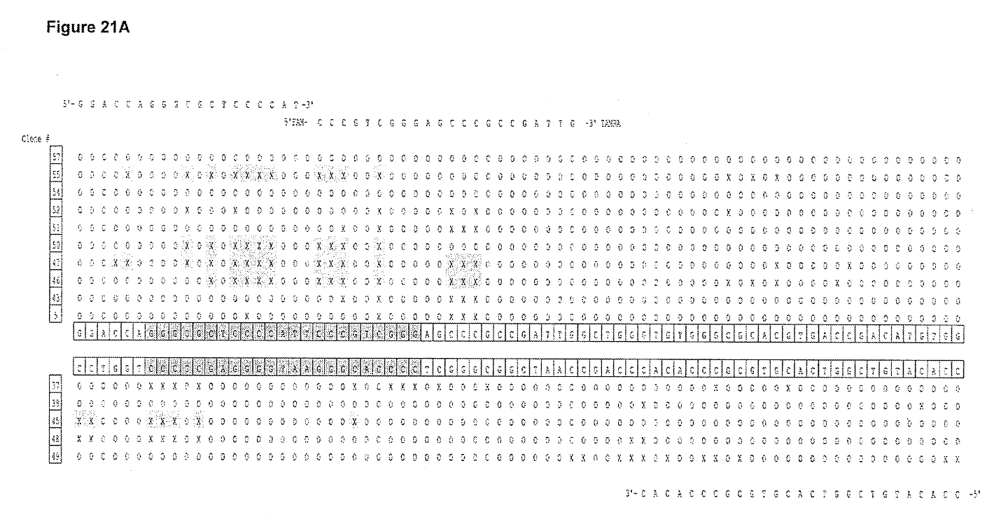

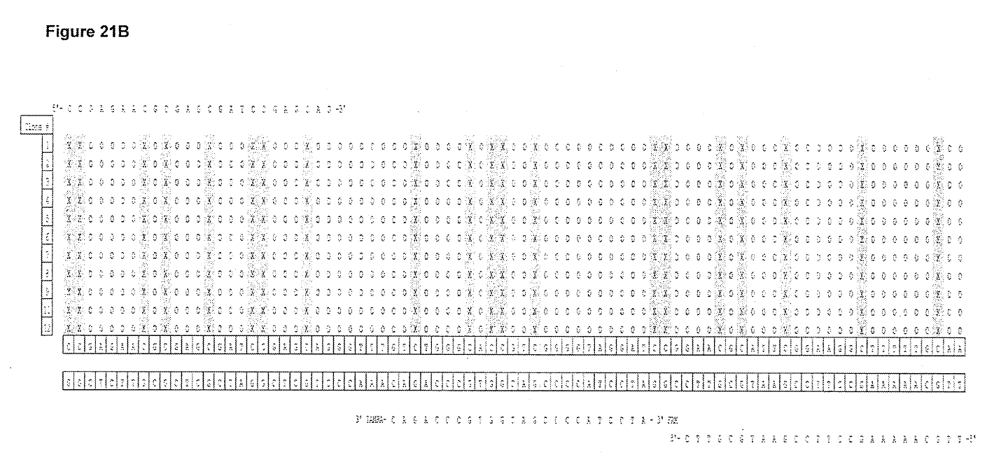

6.11 Unusual Structures Spanning the MS-QPCR Amplicons in the APC and RARB Genes. As can be seen from the data in FIG. 18 the analysis provides a reasonably good fit to the TaqMan® data. Moreover, it suggests that the fluorescence yield could be further lowered by the presence of an unusual structure in the isolated genomic DNA target. Thus, it becomes important to ask whether or not an unusual structure could be detected in the target DNA. For this purpose, we used bisulfite modification of native DNA (Raghavan, 2006) to test for non B-DNA structure at the APC and RARB promoter targets. In this approach, primer sites are chosen so that they lie outside the region of unusual structure. Since the two strands are not complementary after bisulfite modification, the PCR product will contain a mixture of sequences originating from either one strand or the other. FIG. 20 depicts this phenomenon and points out that sequence conversions from each strand can be identified so that clones are thus identified as having originated from one strand or the other. After sequencing of a collection of cloned products the sequences can be arranged so as to depict deamination products originating from the top strand or the bottom strand. The results of an experiment designed to study the larger region spanning the APC control region are depicted in FIG. 21.

6.12. Fifteen clones from a 318 bp region spanning the APC amplicon and eleven clones from a 209 bp region spanning the RARB amplicon were sequenced. In each case the region covered by the MS-QPCR amplicon was extensively deaminated at 55° C. (FIG. 14) demonstrating that each region was accessible to bisulfite modification at this temperature. However, modification was not observed at 37° C. under the same conditions. The results were reproducible, and the same result was obtained with kit-based bisulfite modification or reagent-based modification as described by Raghavan et al. (Raghavan, 2006). The regions accessible to bisulfite at 55° C. span the regions in the RARB and APC genes that are commonly studied in methylation sensitive QPCR (Munson, 2007; Usadel, 2002). In the APC gene the region of bisulfite accessibility spans the binding sites for the forward primer and the TaqMan® probe, while in the RARB gene the region of accessibility spans the entire region.

6.13 Inclusion of 7deazaG in the Reaction. If we assume genomic structure or SSCs involve Hoogsteen pairing as has been suggested for structures thought to occur at many other sites of unusual structure formation in vivo (Raghavan, 2005; Sun, 2005; Lew, 2000), then it is intuitively clear that the QPCR system should show higher yield (i.e. more rapid amplification) when 7deazaGTP is used in place of dGTP in the PCR protocol, providing that the target sequence can be denatured in the primary melt of the amplification cycle. Amplification curves from the APC promoter sequence employing 7deazaGTP were indistinguishable from those employing dGTP (data not shown), suggesting that single-strand conformers do not form at the extension temperature used in the QPCR, that they do not involve Hoogsteen pairing. However, this experiment does not rule out the possibility that Hoogsteen pairing is present in the isolated genomic DNA.

6.14 Using the TaqMan®MS-QPCR System Without Prior Denaturation. All of the currently available MS-QPCR protocols that we are aware of, see (Munson, 2007; Usadel, 2002; Esteller, 2000; Herman, 1996) for example, call for denaturation of the DNA prior to bisulfite treatment. However, the findings we report here show that this step is unnecessary at sequences like those studied above because bisulfite is able to catalyze C→U conversions at the target site without prior denaturation by sodium hydroxide. In general the simplest way to determine whether or not a given system will function properly with this abbreviated procedure is to compare the recovered TaqMan® signal with and without the denaturation step. In the method used here, the sodium hydroxide denaturation step is omitted, and replicate amplification reactions are run in the same rotor in the Rotorgene 3000™. The results obtained with APC, RARB and GSTP1 are summarized in Table 4. In this table the data are expressed as relative Ct values obtained from dilution curves of plasmid DNA standards for each of the expected targets (Munson, 2007). Given equation II above, it is clear that TaqMan® data obtained at sites of this type cannot be expressed in terms of initial target concentration without estimates of the several kinetic constants described in FIG. 17 or evidence that the target in the plasmid standard initially resides in the same structural state present in the genomic DNA.

Both the APC and RARB gene systems behave in a nearly identical fashion with or without the denaturation step. Thus, the interpretation of methylation state is unaltered when the denaturation step is omitted. For example, each of the three genes tested (RARB, APC and GSTPI) would be scored as unmethylated (bold in Table 4) in HK293 cells by either method. This extends the results to the GSTPI gene, which is shown to be accessible to bisulfite without denaturation by the experiment in Table 4. This conclusion is supported by the capacity of the complementary strands of the target duplex to form single-strand conformers (FIG. 16). All of the other decisions regarding methylation state are identical with or without the denaturation step. Based on either method, one would conclude that APC is fully methylated, and that RARB and GSTPI are partially methylated in the PC3 cell line and that all three genes are unmethylated in the HK293.

Except for those targets that did not amplify in a given cell line (Ct>40) there appeared to be a statistically significant (P value<0.05) difference between observed Ct values with and without denaturation, indicating a slight improvement in recovered signal when the denaturation step was omitted. However, calculated fluorescence ratios averaged about 1.18 with a range of 0.32 to 2.47. As a control, we also tested the capacity of the methylated and unmethylated systems to amplify DNA that had not been denatured or bisulfite treated. The mean Ct value for the unmethylated system was 41.97+/−7.79, N=30 while the mean value for the methylated system was 48.05+/−3.97, N=30, suggesting that amplification required bisulfite treatment. The larger standard deviation for the unmethylated system stems from weak apparent amplification from the PC3 cell line.

### Example 7

Reverse Transcription TaqMan® QPCR. RNA Isolation and cDNA Preparation: RNA from EPS sediment was isolated using the RNAqueous Kit (Ambion, Austin, Tex. USA) according to the manufacturer's instructions. Total RNA was added to a 20 μL cDNA reaction containing: 1 U of Omniscript RT (Qiagen, Valencia, Calif., USA), 2 μL 10×RT Buffer, 1 μM random hexamer primers, 0.5 mM dNTPs, and 20 U of Superase-In (Applied Biosystems, Foster City, Calif., USA). The reaction was incubated at 37° C. for one hour. Then 2 μl of the cDNA product was used as template for QPCRs.

### Example 8

Cloning and Isolation of TaqMan® QPCR Standards: Overlap sequences used in cloning standards for PSA, PCA3^{DD3} and TMPRSS2:ERG TaqMan® RNA detection systems were as follows:

PSA: Upper:

[SEQ ID NO: 10]

5′CCTCACAGCTGCCCACTGCATCAGGAACAAAAGCGTGATCTTGCTGGG
PSA: Lower:

[SEQ ID NO: 11]

5′GATGAAACAGGCTGTGCCGACCCAGCAAGATCACGCTTTTGTTCCTG
PCA3DD3Upper:

[SEQ ID NO: 12]

5′CACAGGAAGCACAAAAGGAAGCACAGAGATCCCTGGGAGAAATGCCCG
GCCGCCATCTTGG
PCA3DD3Lower:

[SEQ ID NO: 13]

5′ACAAGCGGGACCAGGCACAGGGCGAGGCTCATCGATGACCCAAGATGG
CGGCCGGGATTT
TMPRSS2: ERG Upper:

[SEQ ID NO: 14]

5′GGGAGCGCCGCCTGGAGCGCGGCAGGAAGCCTTATCAGTTGTGAGTGA
GGAC
TMPRSS2: ERG Lower:

[SEQ ID NO: 15]

5′TTCCGTAGGCACACTCAAACAACGACTGGTCCTCACTCACAACTGATA
AG

The PCR conditions used to produce fragments for blunt end cloning and the cloning vectors used are listed below.

PSA Overlap PCR: 1.65 U Hotstar Taq Polymerase, 1× Hotstar Buffer, 1 mM MgCl₂, 5.0 μl Q solution, 1.08 μM Upper and Lower overlap primers, 0.4 mM dNTPs, in a total reaction volume of 25 μl. Cycling conditions: 1×(95°10 min), 5×(94°30 sec, 60°30 sec, 72°30 sec), 5×(94°30 sec, 58°30 sec, 72°30 sec), 15×(94°30 sec, 56°30 sec, 72°30 sec), 1×(72°3 min). Vector and length: TOPO 2.1, 3999 bp.

PCA3^{DD3} Overlap PC R: 1.65 U Hotstar Taq Polymerase, 1× Hotstar Buffer, 1 mM MgCl₂, 5.0 μl Q solution, 1.08 μM Upper and Lower overlap primers, 400 μM dNTPs, in a total reaction volume of 25 μl. Cycling conditions: 1×(95°10 min), 5×(94°30 sec, 60°30 sec, 72°30 sec), 5×(94°30 sec, 58°30 sec, 72°30 sec), 15×(94°30 sec, 56°30 sec, 72°30 sec), 1×(72°3 min). Vector and length: TOPO 2.1, 4031 bp.

TMPRSS2 Overlap PCR: 1.25 U Hotstar Taq polymerase, 1× Hotstar buffer, 2.5 μl Q solution, 32 μM dNTPs, 0.9 μM Upper and Lower overlap primers, in a final reaction volume of 25 μl. Cycling conditions: 15 cycles: 95°30 sec, 56°30 sec, 72°30 sec. Vector and length: Bluescript, 3041 bp.

GADPH PCR: Cloned from isolated PC3 Cell DNA using 5′ GAAGGTGAAGGTCGGAGT3′ [SEQ ID NO: 16] as forward primer and 5′GAAGATGGTGATGGGATTTG3-[SEQ ID NO: 17] as reverse primers. Reaction Conditions: 1.25 U Hotstar Taq polymerase, 1× Hotstar buffer, 2.5 μl Q solution, 320 μM dNTPs, 0.9 μM forward and reverse primers, in a final reaction volume of 25 μl. Cycling conditions: 95°10 min, 15 cycles: 95°20 sec, 60°40 sec, 72°40 sec.

### Example 9

RT-TaqMan QPCR Conditions: Plasmids containing cloned standards appropriate to each reaction were linearized and serially diluted from stock solutions as previously described above. Standards were run in parallel in the same rotor as the unknowns.

TMPRSS2:ERG: Final reaction conditions: 1.25 U Hotstar Taq polymerase, 1× Hotstar PCR Buffer, 800 μM MgCl₂, 2 μl Q Solution, 0.9 μM Forward and Reverse primers, 320 μM dNTPs, 0.25 μM probe, 2 μl cDNA in a total volume of 25 μl. PCR cycling conditions: 1×(95°10 min); 50×(95°15 sec, 60°30 sec, 72°30 sec).

| | |
|---|---|
| Fwd primer: | |
| 5′-GGAGCGCCGCCTGGAGCG-3′, | [SEQ ID NO: 18] |
| Rev primer: | |
| 5′-TCCGTAGGCACACTCAAACAAC-3′, | [SEQ ID NO: 19] |
| Probe: | |
| Fam-CAGTTGTGAGTGAGGACCAG-BHQ | [SEQ ID NO: 20] |

PSA: Reaction conditions: Final reaction conditions: 1.25 U Hotstar Taq polymerase, 1× Hotstar PCR Buffer, 400 μM MgCl₂, 1 μl Q Solution, 0.9 μM Forward and Reverse primers, 320 μM dNTPs, 0.25 μM probe, 2 μl cDNA in a total volume of 25 μl. PCR cycling conditions: 1×(95°10 min); 50×(95°15 sec, 60°60 sec).

[SEQ ID NO: 21]

| | |
|---|---|
| Fwd primer: | 5′-GATGAAACAGGCTGTGCCG-3′, |

[SEQ ID NO: 22]

| | |
|---|---|
| Rev primer: | 5′-CCTCACAGCTGCCCACTGCA-3′, |

[SEQ ID NO: 23]

| | |
|---|---|
| Probe: | Fam-CAGGAACAAAAGCGTGATCTTGCTGGG-BHQ |

PCA3^{DD3}: Final reaction conditions: 1.25 U Hotstar Taq polymerase, 1× Hotstar PCR Buffer, 400 μM MgCl₂, 1 μl Q Solution, 0.9 μM Forward and Reverse primers, 320 μM dNTPs, 0.25 μM probe, 2 μl cDNA in a total volume of 25 μl. PCR cycling conditions: 1×(95°10 min); 50×(95°20 sec, 56°40 sec, 72°40 sec).

| | |
|---|---|
| Fwd primer: | |
| 5′-AGCACAAAAGGAAGCACAGAGATC-3′, | [SEQ ID NO: 24] |
| Rev primer: | |
| 5′-ACAAGCGGGACCAGGCACAG-3′, | [SEQ ID NO: 25] |
| Probe: | |
| Fam-CATCGATGACCCAAGATGGCGGCC-BHQ | [SEQ ID NO: 26] |

hTERT & GADPH: These reactions we performed in duplex under the following final reaction conditions: 1.25 U Hotstar Taq polymerase, 1× Hotstar PCR Buffer, 800 μM MgCl₂, 2 μl Q Solution, 0.9 μM Forward and Reverse primers, 320 μM dNTPs, 0.25 μM probe, 2 μl cDNA in a total volume of 25 μl. PCR cycling conditions: 1×(95°10 min); 50×(95°20 sec, 60°40 sec, 72°40 sec).

[SEQ ID NO: 27]

| | |
|---|---|
| hTERT Fwd primer: | ′-ACGGCGACATGGAGAACAA-3′, |

[SEQ ID NO: 28]

| | |
|---|---|
| hTERT Rev primer: | 5′-CACTGTCTTCCGCAAGTTCAC-3′, |

[SEQ ID NO: 29]

| | |
|---|---|
| hTERT Probe: | FAM-CTCCTGCGT(dlinternalTAMRA) |
| | TGGTGGATGATTTCTTGTTG. |

[SEQ ID NO: 30]

| | |
|---|---|
| GADH Fwd primer: | 5′-GAAGGTGAAGGTCGGAGT-3′, |

[SEQ ID NO: 31]

| | |
|---|---|
| GADH Rev primer: | 5′-GAAGATGGTGATGGGATTTC-3′, |

[SEQ ID NO: 32]

| | |
|---|---|
| GADH Probe: | Cy5-CAAGCTTCCCGTTCTCAGCC-BHQ3. |

Biostatistical Analysis: Logistic regression models were used to examine the association of biomarker levels with the outcome of biopsy, as well as low or high-grade tumors as measured by biopsy Gleason Sum. For each outcome, four sets of analysis were performed—single biomarker evaluation, baseline serum PSA level and DRE, baseline serum PSA level and DRE plus biomarker evaluation, and various combinations of markers in addition to serum PSA and DRE.

To evaluate the performance of single biomarker, the cut-off points were varied and calculated the true and false positive rates in predicting positive biopsy for PCA (or high grade tumor) based on the value of biomarker. Receiver Operating Characteristic (ROC) curves were then plotted, and the area under the curve (AUC) was calculated by using the Mann-Whitney U-statistic. Confidence intervals were constructed to test whether or not the AUC is significantly different from 0.5, (i.e. the value at which a useless biomarker is defined).

To evaluate the incremental discrimination power of the biomarker over baseline serum PSA and DRE, logistic regression models were constructed with all three variables. The “full” model was then compared to the model with only serum PSA and DRE. The related ROC curve was based on the linear predictor obtained from the logistic model.

The sum of GSTPI, APC, RARB and RASSFI methylated copies was also used as a single marker to evaluate the performance of the combination of methylation markers. This value then entered the logistic regression model together with serum PSA and DRE. On the other hand, the combination of expression markers was based on the linear predictor on PCA3^{DD3} and TMPRSS2:ERG RNA.

When there are two or more continuous markers in the logistic regression model, spline covariate structure for ROC analysis was used, for reasons of flexibility.

To further check whether methylation and expression biomarkers are complementary to each other, a forward stepwise model selection was used starting with the model with just the baseline model containing only PSA and DRE, then individual markers were added to the model if its regression coefficient is statistically significant using AIC criteria. All analyses were carried out in statistical software R 2.4.1.

### Example 10

In certain embodiments, in the TaqMan® QPCR, the data is collected by discrete measurements of the fluorescence at the end of each cycle. Moreover, only one of the two strands (A and B) is monitored. If we assume that this is strand B, then the amount of fluorescence evolved at each cycle can be determined as follows. First, note that:

$$\frac{d [ B ]}{d t} = {k}_{3} [ A ]$$

where [B] is the molar concentration of single-strand random coils of strand B at the beginning of the cycle, [A] is the molar concentration of single-strand random coils of its complementary strand and k₃ is a kinetic rate constant in sec⁻¹ (FIG. 17).

Under the conditions used here, an ideal duplex would be one that dissociated completely to single-strands at 95° C. and then upon rapid cooling to 56° C. would not renature to duplex, or other multi-stranded structures, but rather would combine with the vast excess of primers and Taq polymerase to form initiation complexes that would begin extension once the temperature was rapidly raised to 72° C. The exclusion of multi-stranded structure formation is not unreasonable in the early phases of the amplification since the kinetics of renaturation would be second, third or fourth order for a very dilute target sequence, compared to pseudo first order for primer and Taq polymerase binding.

It is possible that single-strand conformers can form once the temperature is lowered to the annealing temperature. Thus, depending on the sequence, unimolecular folding can compete with primer annealing, probe annealing and Taq Polymerase binding. This process would then act as a molecular sink that would diminish the concentration of available target. The simplest way to address this possibility is to assume the single-strands are in very rapid equilibrium with their respective single-strand conformer. If strand A is in rapid equilibrium with its single-strand conformer (FIG. 17) then:

k₁[A]=k₂[A₈.]

where k₁ and k₂ are rate constants in sec-1, and

$$[ {A}_{ssc} ] = \frac{{k}_{1}}{{k}_{2}} [ A ]$$

Given that:

$${[ AB ]}_{0} = {[ A ]}_{0} = [ A ] + \frac{{k}_{1}}{{k}_{2}} [ A ]$$

where [AB]₀ and [A]₀ are the initial concentrations of the target duplex and target strand A respectively, one sees that

$$[ A ] = {( \frac{{k}_{2}}{{k}_{1} + {k}_{2}} ) [ AB ]}_{0}$$

Similarly, when k₅ and k₆ in sec⁻¹ are the rate constants for the rapid equilibrium between B and its single strand conformer (FIG. 17):

$${k}_{5} [ B ] = {k}_{6} [ {B}_{ssc} ] \\ [ B ] = {( \frac{{k}_{6}}{{k}_{5} + {k}_{6}} ) [ AB ]}_{0}$$

This yields:

$$[ A ] = \frac{( \frac{{k}_{2}}{{k}_{1} + {k}_{2}} )}{( \frac{{k}_{6}}{{k}_{5} + {k}_{6}} )} [ B ]$$

If we define k_{T}_{_{q}} as:

$${k}_{Tq} ≡ \frac{( \frac{{k}_{2}}{{k}_{1} + {k}_{2}} )}{( \frac{{k}_{6}}{{k}_{5} + {k}_{6}} )}$$

$$then \text{:}$$

$${( \frac{d [ B ]}{d t} )}_{n} = {k}_{3} {{k}_{Tq} [ B ] \\ [ B ]}_{t} = {[ B ]}_{{t}_{0}} {e}^{{k}_{3} {k}_{Tq} t}$$

Where [B]ₜ is the concentration of B at time t and [B]ₜ_{₀} is the concentration of B at time zero.

So if we assume that the unusual structure present at the target site in the biological sample can be denatured by the initial heat step in the PCR, and that it refolds to the single-strand conformer with the same equilibrium as the strands produced during the PCR, then we can take:

$${[ B ]}_{{t}_{0}} = {( \frac{{k}_{6}}{{k}_{5} + {k}_{6}} ) [ {AB}_{10} ]}_{0}$$

Then at the end of the time (t) allotted for extension in the first cycle:

$${[ B ]}_{{C}_{1}} = {( \frac{{k}_{6}}{{k}_{5} + {k}_{6}} ) [ AB ]}_{0} {e}^{{k}_{3} {k}_{{T}_{q}} t}$$

If the time allotted for extension in each cycle is constant, the end of the second cycle:

$${[ B ]}_{{C}_{2}} = [ {( \frac{{k}_{6}}{{k}_{5} + {k}_{6}} ) [ AB ]}_{0} {e}^{{k}_{3} {k}_{{T}_{q}} t} ] {e}^{{k}_{3} {k}_{{T}_{q}} t}$$

In the third cycle

$${[ B ]}_{{C}_{3}} = [ [ {( \frac{{k}_{6}}{{k}_{5} + {k}_{6}} ) [ AB ]}_{0} {e}^{{k}_{3} {k}_{{T}_{q}} t} ] {e}^{{k}_{3} {k}_{{T}_{q}} t} ] {e}^{{k}_{3} {k}_{{T}_{q}} t}$$

etc. So that in the Nth cycle:

$${[ B ]}_{{C}_{N}} = {{k}_{0} ( \frac{{k}_{6}}{{k}_{5} + {k}_{6}} ) [ AB ]}_{0} {e}^{{C}_{N} {k}_{3} {k}_{{T}_{q}} t}$$

Since we assume that each molecule of single-strand random coil of B binds one molecule of probe and releases one molecule of chromophore as it is degraded during each cycle, then after the Nth cycle we have:

$$F ( {C}_{N} ) - {F}_{bkd} = ( {{k}_{0} ( \frac{{k}_{6}}{{k}_{5} + {k}_{6}} ) [ AB ]}_{0} - {F}_{bkd} ) {e}^{{C}_{N} {k}_{3} {k}_{{T}_{q}} t}$$

Where F(C_{N}) is the fluorescence produced at the Nth cycle, F_{bkd} is the background fluorescence due to unquenched chromophore on the probe, and k₀ is a machine constant relating chromophore concentration to fluorescence. For baseline corrected data the relationship simplifies to:

$$F ( {C}_{N} ) = ( {{k}_{0} ( \frac{{k}_{6}}{{k}_{5} + {k}_{6}} ) [ AB ]}_{0} ) {e}^{{C}_{N} {k}_{3} {k}_{{T}_{q}} t}$$

Finally, since the chromophores released at each cycle fluoresce during subsequent cycles, the accumulated fluorescence F_{A}(C_{N}) is given by:

$${F}_{A} ( {C}_{N} ) = {\int }_{1}^{{C}_{N}} {{k}_{0} ( \frac{{k}_{6}}{{k}_{5} + {k}_{6}} ) [ AB ]}_{0} {e}^{{C}_{N} {k}_{3} {k}_{{T}_{q}} t} d {C}_{N}$$

$${F}_{A} ( {C}_{N} ) = \frac{{k}_{0}}{{k}_{3} {k}_{{T}_{q}} t} {( \frac{{k}_{6}}{{k}_{5} + {k}_{6}} ) [ AB ]}_{0} {e}^{{C}_{N} {k}_{3} {k}_{{T}_{q}} t} {\vert}_{1}^{{C}_{N}}$$

Since the amount of fluorescence at the first cycle is undetectable, it can be neglected and we have:

$${F}_{A} ( {C}_{N} ) = \frac{{k}_{0}}{{k}_{3} {k}_{{T}_{q}} t} {( \frac{{k}_{6}}{{k}_{5} + {k}_{6}} ) [ AB ]}_{0} {e}^{{C}_{N} {k}_{3} {k}_{{T}_{q}} t} \qquad II$$

It is important to note that when:

$${F}_{0} = \frac{{k}_{0}}{{k}_{3} {k}_{{T}_{q}} t} {( \frac{{k}_{6}}{{k}_{5} + {k}_{6}} ) [ AB ]}_{0}$$

$$and$$

$${k}_{3} {k}_{{T}_{q}} t = ln ( 1 + {E}_{S} )$$

Equation II reduces to the form of the more familiar empirical equation:

F(C_{N})=F₀^{S}(1+E_{S})^{C}^{_{N.}}

### Example 11

Unusual Secondary Structure at a Hot Spot for DNA Methylation in Human Breast Cancer DNA. The human c-Ha-ras-1 gene is subtelomeric on the short arm of chromosome II (Lichter, 1990). Like other telomeric and subtelomeric sequences, it has a very high G+C content, with some regions like that of exon I exceeding 95% G+C (Kasperczyk, 1989). It shares an additional feature with recently identified (de Lange, 1990; Inglehearn 1990) subtelomeric sequences: a tandem array of short GC-rich sequences that are longer and somewhat more complex than the simple hexameric repeat found in the telomere itself. In c-Ha-ras, the tandem array lies 3′ to the coding sequence and is composed of a Variable Number of Tandem Repeats (VNTR). Length variation is easily detected with southern blotting, and individuals can be homozygous or heterozygous for length alleles at this locus.

Southern blots probed with the radiolabed VNTR sequences were used to produce restriction maps of the region surrounding the VNTR. The mapping experiments demonstrate that the restriction enzyme MspI only partially cleaves one or both of the two 5′CCGG sites flanking the c-Ha-ras VNTR in breast cancer specimens and adjacent normal tissue, whereas it is able to completely cleave these same sites in lymphocyte DNA from the same patients. Limit co-digestion controls show that the enzyme was capable of fully cleaving B-DNA from phiX174 in each case.

Standard bisulfite-mediated PCR amplification in which mild sodium hydroxide was used to denature the genomic DNA prior to bisulfite treatment (Herman, 1996) generated amplicons corresponding to regions spanning each of the four single-strands that participate in the two 5′CCGG sites that flank the VNTR. Sequencing of cloned representatives of each amplicon showed that the sites are not mutated or methylated at the 5° C. residue in the breast cancer DNA. 5′CG sites within the MspI recognition sequence and elsewhere in the amplicons exhibit partial methylation. Even so, MspI is known to cleave the observed 5′CCGG or 5′CmCGG sites except when they are single-stranded or present in non-B DNA conformations with single-stranded character.

Additional experiments using non-standard bisulfite-mediated PCR in which amplification is carried out without prior denaturation of the genomic DNA (Raghavan, 2006) showed that the regions reside in a bisulfite-accessible state in isolated breast cancer DNA. Thus, the restriction mapping data and the bisulfite mediated PCR experiments combine to suggest that local non-B DNA or B-DNA hairpin structures within the VNTR region alter the conformation at the VNTR-adjacent MspI sites rendering them refractory to MspI digestion, and that these structures persist in breast cancer DNA and DNA from the surrounding histologically normal tissue.

As stated above, the foregoing is merely intended to illustrate various embodiments of the present invention. The specific modifications discussed above are not to be construed as limitations on the scope of the invention. It will be apparent to one skilled in the art that various equivalents, changes, and modifications may be made without departing from the scope of the invention, and it is understood that such equivalent embodiments are to be included herein. All references cited herein are incorporated by reference as if fully set forth herein.

TABLE 1

| | | | | N |
|---|---|---|---|---|
| | | | N | (Prostate |
| Serum PSA + DRE + Marker | AUC | 95% CI | (Benign) | Cancer) |
| None | 0.630 | (0.491, 0.770) | 33 | 30 |
| Methylated GSTPI Copies | 0.688 | (0.557, 0.820) | 33 | 30 |
| Methylated APC Copies | 0.662 | (0.527, 0.796) | 33 | 30 |
| Methylated RARB Copies | 0.705 | (0.576, 0.835) | 33 | 30 |
| Methylated RASSFI Copies | 0.671 | (0.535, 0.807) | 33 | 30 |
| Sum of Methylaed Copies** | 0.721 | (0.595, 0.847) | 33 | 30 |
| None | 0.645 | (0.519, 0.771) | 39 | 35 |
| PCA3^{DD3} RNA | 0.692 | (0.571, 0.813) | 39 | 35 |
| TMPRSS:ERG RNA | 0.823 | (0.728, 0.919) | 39 | 35 |

*Single marker ROC curves were prepared. The area under the curve (AUC) and its 95% confidence interval are reported for each marker coupled with the baseline covariate markers Serum PSA and DRE.
**The Methylation sum is defined as the sum of all methylated copies at GSTPI, APC, RARB, and RASSFI in a given specimen taken as a single marker.

TABLE 2

| | | | N | N |
|---|---|---|---|---|
| | | | (Gleason's | (Gleason's |
| Serum PSA + DRE + Marker | AUC | 95% CI | Sum <7) | Sum ≧7) |
| None | 0.684 | (0.531, 0.838) | 52 | 11 |
| Methylated GSTPI Copies | 0.701 | (0.551, 0.851) | 52 | 11 |
| Methylated APC Copies | 0.754 | (0.616, 0.892) | 52 | 11 |
| Methylated RARB Copies | 0.764 | (0.618, 0.910) | 52 | 11 |
| Methylated RASSFI Copies | 0.696 | (0.539, 0.852) | 52 | 11 |
| Sum of Methylated Copies** | 0.733 | (0.585, 0.880) | 52 | 11 |
| None | 0.688 | (0.552, 0.824) | 60 | 14 |
| PCA3^{DD3} RNA | 0.751 | (0.638, 0.864) | 60 | 14 |
| TMPRSS:ERG RNA | 0.844 | (0.740, 0.948) | 60 | 14 |

*Single marker ROC curves were prepared and the area under the curve (AUC) and its 95% confidence interval are reported for each marker.
**The Sum of Methylated Copies is defined as the sum of all methylated copies at GSTPI, APC, RARB, and RASSFI in a given specimen taken as a single marker.

TABLE 3

| | Mean (Standard Deviation) | |
|---|---|---|
| □ | Benign | Prostate Cancer |
| Methylated GSTPI Copies | 1.54 (2.27) | 1.26 (1.68) |
| Methylated APC Copies | 0.81 (1.67) | 2.53 (7.97) |
| Methylated RARB Copies | 13.86 (19.39) | 43.66 (79.60) |
| Methylated RASSFI Copies | 1.85 (3.12) | 4.27 (6.14) |
| *Sum of Methylated Copies | | |
| PCA3^{DD3} Copies | 3188.95 (6946.72) | 5557.66 (8847.53) |
| TMPRSS2:ERG Copies | 33.76 (31.09) | 75.15 (60.90) |

*The Sum of Methylated Copies is defined as the sum of all methylated copies at GSTPI, APC, RARB, and RASSFI in a given specimen taken as a single marker.

TABLE 4
RARB

| Cell Line | TaqMan ® System | *Ct(D) +/− STD | Ct(N) +/− STD | ΔCt +/− 2STD | P value | **N | 1 + E | †F(N)/F(D) |
|---|---|---|---|---|---|---|---|---|
| HK293 | Unmethylated | 30.49 +/− 0.28 | 29.98 +/− 0.38 | −0.51 +/− 0.49 | 0.0421 | 5 | 1.92 | 0.719 |
| | Methylated | 48.34 +/− 3.70 | 50.00 +/− 0.00 | −1.66 +/− 3.81 | 0.3451 | 5 | 1.98 | 0.322 |
| PC3 | Unmethylated | 31.99 +/− 0.35 | 33.13 +/− 0.19 | −1.14 +/− 0.44 | 0.0002 | 5 | 1.94 | 0.475 |
| | Methylated | 27.34 +/− 0.15 | 26.48 +/− 0.11 | 0.86 +/− 0.30 | 0.0001 | 5 | 1.98 | 1.768 |

GSTP1

| Cell Line | TaqMan ® System | Ct(D) +/− STD | Ct(N) +/− STD | ΔCt +/− 2STD | P value | N | 1 + E | F(N)/F(D) |
|---|---|---|---|---|---|---|---|---|
| HK293 | Unmethylated | 32.88 +/− 0.10 | 32.43 +/− 0.42 | 0.45 +/− 0.45 | 0.0481 | 5 | 1.08 | 1.035 |
| | Methylated | 46.11 +/− 3.70 | 46.47 +/− 4.90 | −0.36 +/− 6.33 | 0.8989 | 5 | 1.08 | 0.973 |
| PC3 | Unmethylated | 31.59 +/− 0.21 | 31.34 +/− 0.06 | 0.25 +/− 0.23 | 0.0337 | 5 | 2.10 | 1.204 |
| | Methylated | 36.72 +/− 0.21 | 35.50 +/− 0.25 | 1.22 +/− 0.36 | 0.0001 | 5 | 2.10 | 2.472 |

APC

| Cell Line | TaqMan ® System | Ct(D) +/− STD | Ct(N) +/− STD | ΔCt +/− 2STD* | P value | N | 1 + E | F(N)/F(D) |
|---|---|---|---|---|---|---|---|---|
| HK293 | Unmethylated | 29.67 +/− 0.03 | 29.11 +/− 0.31 | 0.56 +/− 0.32 | 0.0038 | 5 | 1.06 | 1.033 |
| | Methylated | 50.00 +/− 0.00 | 50.00 +/− 0.00 | 0.00 +/− 0.00 | N.A | 5 | 1.06 | 1.000 |
| PC3 | Unmethylated | 50.00 +/− 0.00 | 49.95 +/− 0.11 | 0.05 +/− 0.11 | 0.3392 | 5 | 1.94 | 1.034 |
| | Methylated | 27.67 +/− 0.09 | 26.53 +/− 0.31 | 1.14 +/− 0.33 | 0.0001 | 5 | 1.94 | 2.129 |

*Ct(D): Ct value obtained for NaOH denatured DNA; Ct(N) Ct value obtained for native DNA.
ΔCt = Ct(N) − Ct(D).
**N = number of replicate assays.
†F(N)/F(D) = (1 + E)\hat{ }ΔCt. Average F(N)/F(D) = 1.180 +/− 0.640 for all data.

TABLE 5

| | | | | N |
|---|---|---|---|---|
| | | | N | (Prostate |
| Marker | AUC | 95% CI | (Benign) | Cancer) |
| Methylated GSTPI Copies | 0.523 | (0.383, 0.663) | 33 | 30 |
| Methylated APC Copies | 0.560 | (0.430, 0.689) | 33 | 30 |
| Methylated RARB Copies | 0.598 | (0.455, 0.741) | 33 | 30 |
| Methylated RASSFI Copies | 0.641 | (0.504, 0.777) | 33 | 30 |
| Sum of Methylated Copies** | 0.576 | (0.432, 0.720) | 33 | 30 |
| GADH RNA | 0.526 | (0.389, 0.663) | 39 | 35 |
| RT-PSA RNA | 0.509 | (0.375, 0.643) | 39 | 35 |
| PCA3^{DD3} RNA | 0.600 | (0.469, 0.732) | 39 | 35 |
| TMPRSS:ERG RNA | 0.778 | (0.671, 0.886) | 39 | 35 |

*Single marker ROC curves were prepared and the area under the curve (AUC) and its 95% confidence interval are reported for each marker.
**The Methylation sum is defined as the sum of all methylated copies at GSTPI, APC, RARB, and RASSFI in a given specimen taken as a single marker.

### REFERENCES

1. Lichter, P., Tang, C. J., Call, K., Hermanson, G., Evans, G. A., Housman, D. and Ward, D. C. (1990) High-resolution mapping of human chromosome II by in situ hybridization with cosmid clones. Science, 247, 64-69.
2. Kasperczyk, A., Mermer, B. A., Parkinson, D. R., Lonergan, J. A. and Krontiris, T. G. (1989) Allele-specific deletion in exon I of the HRAS1 gene. Am J Hum Genet, 45, 689-696.
3. de Lange, T., Shiue, L., Myers, R. M., Cox, D. R., Naylor, S. L., Killery, A. M. and Varmus, H. E. (1990) Structure and variability of human chromosome ends. Mol Cell Biol, 10, 518-527.
4. Inglehearn, C. F. and Cooke, H. J. (1990) A VNTR immediately adjacent to the human pseudoautosomal telomere. Nucleic Acids Res, 18, 471-476.
5. Herman, J. G., Graff, J. R., Myohanen, S., Nelkin, B. D. and Baylin, S. B. (1996) Methylation-specific PCR: a novel PCR assay for methylation status of CpG islands. Proc Natl Acad Sci USA, 93, 9821-9826.
6. Raghavan, S. C., Tsai, A., Hsieh, C. L. and Lieber, M. R. (2006) Analysis of non-B DNA structure at chromosomal sites in the mammalian genome. Methods Enzymol, 409, 301-316.
7. Robertson, K. D., Ait-Si-Ali, S., Yokochi, T., Wade, P. A., Jones, P. L. and Wolffe, A. P. (2000) DNMT1 forms a complex with Rb, E2F1 and HDAC1 and represses transcription from E2F-responsive promoters. Nat Genet, 25, 338-342.
8. Rountree, M. R., Bachman, K. E. and Baylin, S. B. (2000) DNMT1 binds HDAC2 and a new co-repressor, DMAP1, to form a complex at replication foci. Nat Genet, 25, 269-277.
9. Macleod, D., Charlton, J., Mullins, J. and Bird, A. P. (1994) Sp1 sites in the mouse part gene promoter are required to prevent methylation of the CpG island. Genes Dev, 8, 2282-2292.
10. Brandeis, M., Frank, D., Keshet, I., Siegfried, Z., Mendelsohn, M., Nemes, A., Temper, V., Razin, A. and Cedar, H. (1994) Sp1 elements protect a CpG island from de novo methylation. Nature, 371, 435-438.
11. Smith, S. S. (1991) DNA methylation in eukaryotic chromosome stability. Mol Carcinog, 4, 91-92.
12. Smith, S. S, and Crocitto, L. E. (1999) DNA Methylation in Eukaryotic Chromosome Stability Revisited: DNA Methyltransferase in the Management of DNA Conformation Space. Mol. Carcinog., 26, 1-9.
13. Carro, S., Bergo, A., Mengoni, M., Bachi, A., Badaracco, G., Kilstrup-Nielsen, C. and Landsberger, N. (2004) A novel protein, Xenopus p20, influences the stability of MeCP2 through direct interaction. J Biol Chem, 279, 25623-25631.
14. Patra, S. K., Patra, A., Zhao, H., Carroll, P. and Dahiya, R. (2003) Methyl-CPG-DNA binding proteins in human prostate cancer: expression of CXXC sequence containing MBD1 and repression of MBD2 and MeCP2. Biochem Biophys Res Commun, 302, 759-766.
15. Jones, P. A. and Baylin, S. B. (2007) The epigenomics of cancer. Cell, 128, 683-692.
16. Reynolds, M. A., Kastury, K., Groskopf, J., Schalken, J. A. and Rittenhouse, H. (2007) Molecular markers for prostate cancer. Cancer Lett, 249, 5-13.
17. Marks, L. S., Fradet, Y., Deras, I. L., Blase, A., Mathis, J., Aubin, S. M., Cancio, A. T., Desaulniers, M., Ellis, W. J., Rittenhouse, H. et al. (2007) PCA3 molecular urine assay for prostate cancer in men undergoing repeat biopsy. Urology, 69, 532-535.
18. van Gils, M. P., Cornel, E. B., Hessels, D., Peelen, W. P., Witjes, J. A., Mulders, P. F., Rittenhouse, H. G. and Schalken, J. A. (2007) Molecular PCA3 diagnostics on prostatic fluid. Prostate, 67, 881-887.
19. Perner, S., Demichelis, F., Beroukhim, R., Schmidt, F. H., Mosquera, J. M., Setlur, S., Tchinda, J., Tomlins, S. A., Hofer, M. D., Pienta, K. G. et al. (2006) TMPRSS2:ERG Fusion-Associated Deletions Provide Insight into the Heterogeneity of Prostate Cancer. Cancer Res, 66, 8337-8341.
20. Tomlins, S. A., Rhodes, D. R., Perner, S., Dhanasekaran, S. M., Mehra, R., Sun, X. W., Varambally, S., Cao, X., Tchinda, J., Kuefer, R. et al. (2005) Recurrent fusion of TMPRSS2 and ETS transcription factor genes in prostate cancer. Science, 310, 644-648.
21. Laxman, B., Tomlins, S. A., Mehra, R., Morris, D. S., Wang, L., Helgeson, B. E., Shah, R. B., Rubin, M. A., Wei, J. T. and Chinnaiyan, A. M. (2006) Noninvasive detection of TMPRSS2:ERG fusion transcripts in the urine of men with prostate cancer. Neoplasia, 8, 885-888.
22. Attard, G., Clark, J., Ambroisine, L., Fisher, G., Kovacs, G., Flohr, P., Berney, D., Foster, C. S., Fletcher, A., Gerald, W. L. et al. (2007) Duplication of the fusion of TMPRSS2 to ERG sequences identifies fatal human prostate cancer. Oncogene.
23. Stark, G. R., Debatisse, M., Giulotto, E. and Wahl, G. M. (1989) Recent progress in understanding mechanisms of mammalian DNA amplification. Cell, 57, 901-908.
24. Singer, M. J., Mesner, L. D., Friedman, C. L., Trask, B. J. and Hamlin, J. L. (2000) Amplification of the human dihydrofolate reductase gene via double minutes is initiated by chromosome breaks. Proc Natl Acad Sci USA, 97, 7921-7926.
25. Munson, K., Clark, J., Lamparska-Kupsik, K. and Smith, S. S. (2007) Recovery of Bisulfite-Converted Genomic Sequences in the Methylation-Sensitive QPCR. Nucleic Acids Research, 35, 2893-2903. doi: 10.1093/nar/gkm055.
26. Hoque, M. O., Topaloglu, O., Begum, S., Henrique, R., Rosenbaum, E., Van Criekinge, W., Westra, W. H. and Sidransky, D. (2005) Quantitative methylation-specific polymerase chain reaction gene patterns in urine sediment distinguish prostate cancer patients from control subjects. J Clin Oncol, 23, 6569-6575.
27. Wang, J., Cai, Y., Ren, C. and Ittmann, M. (2006) Expression of variant TMPRSS2/ERG fusion messenger RNAs is associated with aggressive prostate cancer. Cancer Res, 66, 8347-8351.
28. Mills, D. R., Peterson, R. L. and Spiegelman, S. (1967) An extracellular Darwinian experiment with a self-duplicating nucleic acid molecule. Proc Natl Acad Sci USA, 58, 217-224.
29. Irvine, D., Tuerk, C. and Gold, L. (1991) SELEXION. Systematic evolution of ligands by exponential enrichment with integrated optimization by non-linear analysis. J. Mol. Biol., 222, 739-761.
30. Paul, N., Springsteen, G. and Joyce, G. F. (2006) Conversion of a ribozyme to a deoxyribozyme through in vitro evolution. Chem Biol, 13, 329-338.
31. Jones, P. A. (2005) Overview of cancer epigenetics. Semin. Hematol., 42, S3-S8.
32. Baylin, S. B. and Ohm, J. E. (2006) Epigenetic gene silencing in cancer—a mechanism for early oncogenic pathway addiction? Nat. Rev. Cancer, 6, 107-116.
33. Issa, J. P. (2000) CpG-island methylation in aging and cancer. Curr. Top. Microbiol. Immunol., 249, 101-118.
34. Waalwijk, C. and Flavell, R. A. (1978) DNA methylation at a CCGG sequence in the large intron of the rabbit beta-globin gene: tissuespecific variations. Nucleic Acids Res., 5, 4631-4634.
35. Wilson, D. S, and Szostak, J. W. (1999) In vitro selection of functional nucleic acids. Annu Rev Biochem, 68, 611-647.
36. Smith, S. S, and Crocitto, L. E. (1999) DNA Methylation in Eukaryotic Chromosome Stability Revisited: DNA Methyltransferase in the Management of DNA Conformation Space. Mol. Carcinog., 26, 1-9.
37. Liu, Z., Lee, A. and Gilbert, W. (1995) Gene disruption of a G4-DNA-dependent nuclease in yeast leads to cellular senescence and telomere shortening. Proc Natl Acad Sci USA, 92, 6002-6006.
38. Smith, S. S., Laayoun, A., Lingeman, R. G., Baker, D. J. and Riley, J. (1994) Hypermethylation of telomere-like foldbacks at codon 12 of the human c-Ha-ras gene and the trinucleotide repeat of the FMR-1 gene of fragile X. J Mol Biol, 243, 143-151.
39. Mitas, M., Yu, A., Dill, J., Kamp, T. J., Chambers, E. J. and Haworth, I. S. (1995) Hairpin properties of single-stranded DNA containing a GC-rich triplet repeat: (CTG)15. Nucleic Acids Res, 23, 1050-1059.
40. Chen, X., Mariappan, S. V., Catasti, P., Ratliff, R., Moyzis, R. K., Laayoun, A., Smith, S. S., Bradbury, E. M. and Gupta, G. (1995) Hairpins are formed by the single DNA strands of the fragile X triplet repeats: structure and biological implications. Proc Natl Acad Sci USA, 92, 5199-5203.
41. Mitas, M., Yu, A., Dill, J. and Haworth, I. S. (1995) The trinucleotide repeat sequence d(CGG)15 forms a heat-stable hairpin containing Gsyn. Ganti base pairs. Biochemistry, 34, 12803-12811.
42. Kovtun, I. V. and McMurray, C. T. (2001) Trinucleotide expansion in haploid germ cells by gap repair. Nat Genet, 27, 407-411.
43. Gacy, A. M., Goellner, G., Juranic, N., Macura, S, and McMurray, C. T. (1995) Trinucleotide repeats that expand in human disease form hairpin structures in vitro. Cell, 81, 533-540.
44. Darlow, J. M. and Leach, D. R. (1998) Evidence for two preferred hairpin folding patterns in d(CGG). d(CCG) repeat tracts in vivo. J Mol Biol, 275, 17-23.
45. Darlow, J. M. and Leach, D. R. (1998) Secondary structures in d(CGG) and d(CCG) repeat tracts. J Mol Biol, 275, 3-16.
46. Raghavan, S. C., Chastain, P., Lee, J. S., Hegde, B. G., Houston, S., Langen, R., Hsieh, C. L., Haworth, I. S, and Lieber, M. R. (2005) Evidence for a triplex DNA conformation at the bcl-2 major breakpoint region of the t(14; 18) translocation. J Biol Chem, 280, 22749-22760.
47. Antequera, F. and Bird, A. (1993) Number of CpG islands and genes in human and mouse. Proc Natl Acad Sci USA, 90, 11995-11999.
48. Trinklein, N. D., Aldred, S. J., Saldanha, A. J. and Myers, R. M. (2003) Identification and functional analysis of human transcriptional promoters. Genome Res, 13, 308-312.
49. Sun, D., Guo, K., Rusche, J. J. and Hurley, L. H. (2005) Facilitation of a structural transition in the polypurine/polypyrimidine tract within the proximal promoter region of the human VEGF gene by the presence of potassium and G-quadruplex-interactive agents. Nucleic Acids Res, 33, 6070-6080.
50. Lew, A., Rutter, W. J. and Kennedy, G. C. (2000) Unusual DNA structure of the diabetes susceptibility locus IDDM2 and its effect on transcription by the insulin promoter factor Pur-1/MAZ. Proc Natl Acad Sci USA, 97, 12508-12512.
51. Haga, K., Fujita, H., Nomoto, M., Sazawa, A., Nakagawa, K., Harabayashi, T., Shinohara, N., Takimoto, M., Nonomura, K. and Kuzumaki, N. (2004) Gelsolin gene silencing involving unusual hypersensitivities to dimethylsulfate and KMnO4 in vivo footprinting on its promoter region. Int J Cancer, 111, 873-880.
52. Ackerman, S. L., Minden, A. G. and Yeung, C. Y. (1993) The minimal self-sufficient element in a murine G+C-rich promoter is a large element with imperfect dyad symmetry. Proc Natl Acad Sci USA, 90, 11865-11869.
53. Simonsson, T., Pecinka, P. and Kubista, M. (1998) DNA tetraplex formation in the control region of c-myc. Nucleic Acids Res, 26, 1167-1172.
54. Smith, S. S, and Baker, D. J. (1997) Stalling of human methyltransferase at single-strand conformers from the Huntington's locus. Biochem Biophys Res Commun, 234, 73-78.
55. Fackler, M. J., McVeigh, M., Mehrotra, J., Blum, M. A., Lange, J., Lapides, A., Garrett, E., Argani, P. and Sukumar, S. (2004) Quantitative multiplex methylation-specific PCR assay for the detection of promoter hypermethylation in multiple genes in breast cancer. Cancer Res, 64, 4442-4452.
56. Harden, S. V., Sanderson, H., Goodman, S. N., Partin, A. A., Walsh, P. C., Epstein, J. I. and Sidransky, D. (2003) Quantitative GSTP1 methylation and the detection of prostate adenocarcinoma in sextant biopsies. J Natl Cancer Inst, 95, 1634-1637.
57. Dulaimi, E., Uzzo, R. G., Greenberg, R. E., Al-Saleem, T. and Cairns, P. (2004) Detection of bladder cancer in urine by a tumor suppressor gene hypermethylation panel. Clin Cancer Res, 10, 1887-1893.
58. Henke, W., Herdel, K., Jung, K., Schnorr, D. and Loening, S. A. (1997) Betaine improves the PCR amplification of GC-rich DNA sequences. Nucleic Acids Res, 25, 3957-3958.
59. Dierick, H., Stul, M., De Kelver, W., Marynen, P. and Cassiman, J. J. (1993) Incorporation of dITP or 7-deaza dGTP during PCR improves sequencing of the product. Nucleic Acids Res, 21, 4427-4428.
60. Jung, A., Ruckert, S., Frank, P., Brabletz, T. and Kirchner, T. (2002) 7-Deaza-2′-deoxyguanosine allows PCR and sequencing reactions from CpG islands. Mol Pathol, 55, 55-57.
61. Musso, M., Bocciardi, R., Parodi, S., Ravazzolo, R. and Ceccherini, I. (2006) Betaine, dimethyl sulfoxide, and 7-deaza-dGTP, a powerful mixture for amplification of GC-rich DNA sequences. J Mol Diagn, 8, 544-550.
62. Salisbury, S. A., Wilson, S. E., Powell, H. R., Kennard, O., Lubini, P., Sheldrick, G. M., Escaja, N., Alazzouzi, E., Grandas, A. and Pedroso, E. (1997) The bi-loop, a new general four-stranded DNA motif. Proc Natl Acad Sci USA, 94, 5515-5518.
63. Smith, S. S. (2000) Gilbert's conjecture: the search for DNA (cytosine-5) demethylases and the emergence of new functions for eukaryotic DNA (cytosine-5) methyltransferases. J. Mol. Biol., 302, 1-7.
64. Shevchuk, T., Kretzner, L., Munson, K., Axume, J., Clark, J., Dyachenko, O. V., Caudill, M., Buryanov, Y. and Smith, S. S. (2005) Transgene-induced CCWGG methylation does not alter CG methylation patterning in human kidney cells. Nucleic Acids Res, 33, 6124-6136.
65. Chen, Z. X. and Riggs, A. D. (2005) Maintenance and regulation of DNA methylation patterns in mammals. Biochem. Cell Biol., 83, 438-448.
66. Sundquist, W. I. and Klug, A. (1989) Telomeric DNA dimerizes by formation of guanine tetrads between hairpin loops. Nature, 342, 825-829.
67. Sen, D. and Gilbert, W. (1990) A sodium-potassium switch in the formation of four-stranded G4-DNA. Nature, 344, 410-414.
68. Ramakers, C., Ruijter, J. M., Deprez, R. H. and Moorman, A. F. (2003) Assumption-free analysis of quantitative real-time polymerase chain reaction (PCR) data. Neurosci Lett, 339, 62-66.
69. Peirson, S. N., Butler, J. N. and Foster, R. G. (2003) Experimental validation of novel and conventional approaches to quantitative real-time PCR data analysis. Nucleic Acids Res, 31, e73.
70. Shen, L., Guo, Y., Chen, X., Ahmed, S, and Issa, J. P. (2007) Optimizing annealing temperature overcomes bias in bisulfite PCR methylation analysis. Biotechniques, 42, 48, 50, 52 passim.
71. Wojdacz, T. K. and Dobrovic, A. (2007) Methylation-sensitive high resolution melting (MS-HRM): a new approach for sensitive and high-throughput assessment of methylation. Nucleic Acids Res, 35, e41.
72. Usadel, H., Brabender, J., Danenberg, K. D., Jeronimo, C., Harden, S., Engles, J., Danenberg, P. V., Yang, S, and Sidransky, D. (2002) Quantitative adenomatous polyposis coli promoter methylation analysis in tumor tissue, serum, and plasma DNA of patients with lung cancer. Cancer Res, 62, 371-375.
73. Esteller, M., Sparks, A., Toyota, M., Sanchez-Cespedes, M., Capella, G., Peinado, M. A., Gonzalez, S., Tarafa, G., Sidransky, D., Meltzer, S. J. et al. (2000) Analysis of adenomatous polyposis coli promoter hypermethylation in human cancer. Cancer Res, 60, 4366-4371.
74. Herman, J. G., Graff, J. R., Myohanen, S., Nelkin, B. D. and Baylin, S. B. (1996) Methylation-specific PCR: a novel PCR assay for methylation status of CpG islands. Proc Natl Acad Sci USA, 93, 9821-9826.
75. Raghavan, S. C., Swanson, P. C., Wu, X., Hsieh, C. L. and Lieber, M. R. (2004) A non-B-DNA structure at the Bcl-2 major breakpoint region is cleaved by the RAG complex. Nature, 428, 88-93.
76. Smith, S. S., Niu, L., Baker, D. J., Wendel, J. A., Kane, S. E. and Joy, D. S. (1997) Nucleoprotein-based nanoscale assembly. Proc Natl Acad Sci USA, 94, 2162-2167.
77. Tostesen, E., Jerstad, G. I. and Hovig, E. (2005) Stitchprofiles.uio.no: analysis of partly melted DNA conformations using stitch profiles. Nucleic Acids Res, 33, W573-576.
78. Siddiqui-Jain, A., Grand, C. L., Bearss, D. J. and Hurley, L. H. (2002) Direct evidence for a G-quadruplex in a promoter region and its targeting with a small molecule to repress c-MYC transcription. Proc Natl Acad Sci USA, 99, 11593-11598.
79. Mathur, V., Verma, A., Maiti, S, and Chowdhury, S. (2004) Thermodynamics of i-tetraplex formation in the nuclease hypersensitive element of human c-myc promoter. Biochem Biophys Res Commun, 320, 1220-1227.
80. Simonsson, T., Pribylova, M. and Vorlickova, M. (2000) A nuclease hypersensitive element in the human c-myc promoter adopts several distinct i-tetraplex structures. Biochem Biophys Res Commun, 278, 158-166.
81. Blossey, B. and Carlon, E. (2003) Reparametrizing loop entropy weights: Effect on DNA melting curves. Phys. Rev. E 68, 061911.
82. Razin, A. and Riggs, A. D. (1980) DNA Methylation and Gene Function, Science, 210, 604-610.
83. Xiong, Z. and Laird, P. W. (1997) COBRA: a sensitive and quantitative DNA methylation assay. Nucleic Acids Res., 25, 2532-2534.
84. Church, G. M. and Gilbert, W. (1984) Genomic sequencing. Proc. Natl. Acad. Sci. USA, 81, 1991-1995.
85. Saluz, H. and Jost, J. P. (1989) A simple high-resolution procedure to study DNA methylation and in vivo DNA-protein interactions on a single-copy gene level in higher eukaryotes. Proc. Natl. Acad. Sci. USA, 86, 2602-2606.
86. Pfeifer, G. P., Steigerwald, S. D., Mueller, P. R., Wold, B. and Riggs, A. D. (1989) Genomic sequencing and methylation analysis by ligation mediated PCR.
Science, 246, 810-813.
87. Rein, T., Natale, D. A., Gartner, U., Niggemann, M., DePamphilis, M. L. and Zorbas, H. (1997) Absence of an unusual ‘densely methylated island’ at the hamster dhfr ori-beta. J. Biol. Chem., 272, 10021-10029.
88. Frommer, M., McDonald, L. E., Millar, D. S., Collis, C. M., Watt, F., Grigg, G. W., Molloy, P. L. and Paul, C. L. (1992) A genomic sequencing protocol that yields a positive display of 5-methylcytosine residues in individual DNA strands. Proc. Natl. Acad. Sci. USA, 89, 1827-1831.
89. Warnecke, P. M., Stirzaker, C., Song, J., Grunau, C., Melki, J. R. and Clark, S. J. (2002) Identification and resolution of artifacts in bisulfite sequencing. Methods, 27, 101-107.
90. Hayatsu, H., Wataya, Y., Kai, K. and lida, S. (1970) Reaction of sodium bisulfite with uracil, cytosine, and their derivatives. Biochemistry, 9, 2858-2865.
91. Hayatsu, H., Wataya, Y. and Kazushige, K. (1970) The addition of sodium bisulfite to uracil and to cytosine. J. Am. Chem. Soc., 92, 724-726.
92. Shiraishi, M. and Hayatsu, H. (2004) High-speed conversion of cytosine to uracil in bisulfite genomic sequencing analysis of DNA methylation. DNA Res., 11, 409-415.
93. Wang, R. Y., Gehrke, C. W. and Ehrlich, M. (1980) Comparison of bisulfite modification of 5-methyldeoxycytidine and deoxycytidine residues. Nucleic Acids Res., 8, 4777-4790.
94. Suzuki, T., Ohsumi, S, and Makino, K. (1994) Mechanistic studies on depurination and apurinic site chain breakage in oligodeoxyribonucleotides. Nucleic Acids res., 22, 4997-5003.
95. Grunau, C., Clark, S. J. and Rosenthal, A. (2001) Bisulfite genomic sequencing: systematic investigation of critical experimental parameters. Nucleic Acids Res., 29, e65.
96. Gonzalgo, M. L. and Jones, P. A. (1997) Rapid quantitation of methylation differences at specific sites using methylation-sensitive single nucleotide primer extension (Ms-SNuPE). Nucleic Acids Res., 25, 2529-2531.
97. Smith, S. S., Gilroy, T. E. and Ferrari, F. A. (1983) The influence of agarose-DNA affinity on the electrophoretic separation of DNA fragments in agarose gels. Anal. Biochem., 128, 138-151.
98. Fuller, R. A., Clark, J., Kretzner, L., Korns, D., Blair, S. L., Crocitto, L. E. and Smith, S. S. (2003) Use of microfluidics chips for the detection of human telomerase RNA. Anal. Biochem., 313, 331-334.
99. Clark, J., Shevchuk, T., Swiderski, P. M., Dabur, R., Crocitto, L. E., Buryanov, Y. I. and Smith, S. S. (2003) Mobility-shift analysis with microfluidics chips. Biotechniques, 35, 548-554.
100. Brena, R. M., Auer, H., Kornacker, K., Hackanson, B., Raval, A., Byrd, J. C. and Plass, C. (2006) Accurate quantification of DNA methylation using combined bisulfite restriction analysis coupled with the Agilent 2100 Bioanalyzer platform. Nucleic Acids Res., 34, e17.
101. Hoque, M. O., Rosenbaum, E., Westra, W. H., Xing, M., Ladenson, P., Zeiger, M. A., Sidransky, D. and Umbricht, C. B. (2005) Quantitative assessment of promoter methylation profiles in thyroid neoplasms. J. Clin. Endocrinol. Metab., 90, 4011-4018.
102. Dulaimi, E., Uzzo, R. G., Greenberg, R. E., Al-Saleem, T. and Cairns, P. (2004) Detection of bladder cancer in urine by a tumor suppressor gene hypermethylation panel. Clin. Cancer. Res., 10, 1887-1893.
103. Bastian, P. J., Palapattu, G. S., Lin, X., Yegnasubramanian, S., Mangold, L. A., Trock, B., Eisenberger, M. A., Partin, A. W. and Nelson, W. G. (2005) Preoperative serum DNA GSTP1 CpG island hypermethylation and the risk of early prostate-specific antigen recurrence following radical prostatectomy. Clin. Cancer. Res., 11, 4037-4043.
104. Botchan, M., McKenna, G. and Sharp, P. A. (1974) Cleavage of mouse DNA by a restriction enzyme as a clue to the arrangement of genes. Cold Spring Harb Symp. Quant. Biol., 38, 383-395.
105. Hamer, D. H. and Thomas, C. A. Jr. (1975) The cleavage of Drosophila melanogaster DNA by restriction endonucleases. Chromosoma, 49, 243-267.
106. Boyd, V. L. and Zon, G. (2004) Bisulfite conversion of genomic DNA for methylation analysis: protocol simplification with higher recovery applicable to limited samples and increased throughput. Anal. Biochem., 326, 278-280.
107. Olek, A., Oswald, J. and Walter, J. (1996) A modified and improved method for bisulphite based cytosine methylation analysis. Nucleic Acids Res., 24, 5064-5066.
108. Wang, Y., Zheng, W., Luo, J., Zhang, D. and Zuhong, L. (2006) In situ bisulfite modification of membrane-immobilized DNA for multiple methylation analysis. Anal. Biochem., 359, 183-188.
109. Toyooka, K. O., Toyooka, S., Maitra, A., Feng, Q., Kiviat, N. C., Smith, A., Minna, J. D., Ashfaq, R. and Gazdar, A. F. (2002) Establishment and validation of real-time polymerase chain reaction method for CDH1 promoter methylation. Am. J. Pathol., 161, 629-634.
110. Goessl, C., Muller, M., Heicappell, R., Krause, H., Straub, B., Schrader, M. and Miller, K. (2001) DNA-based detection of prostate cancer in urine after prostatic massage. Urology, 58, 335-338.
111. Gonzalgo, M. L., Paylovich, C. P., Lee, S. M. and Nelson, W. G. (2003) Prostate cancer detection by GSTP1 methylation analysis of postbiopsy urine specimens. Clin Cancer Res, 9, 2673-2677.

1-32. (canceled)

33. A method of diagnosing a prostate cell proliferative disorder by detecting one or more preselected methylation biomarker in a subject comprising:
obtaining a biological sample from the subject;
isolating DNA from the sample;
contacting the isolated DNA or fragment thereof with one or more methylation-sensitive reagent, wherein the treated DNA is optionally PCR amplified; and
determining the presence of one or a combination of methylation biomarker sequences selected from the group consisting of: PSA RNA; TMPRSS2:ERG RNA; GSTPI, APC, RARB, RASSFI DNA; TMPRSS2:ERG Type III or VI fusion RNA; GSTPI, APC, RARB, RASSFI and PCA3.

34. The method of claim 33 further comprising:
obtaining a prostatic secretion (EPS) sample from the subject and measuring the level of a preselected biomarker in the EPS;
obtaining a prostate specific antigen sample from the subject and measuring the level of PSA in the sample;
wherein the presence of a EPS biomarker, and an elevated level of PSA as compared to the level of PSA in a normal subject indicates the presence of a prostate cell proliferative disorder.

35. The method of claim 33, wherein the DNA is genomic DNA.

36. The method of claim 33, wherein the prostate cell proliferative disorder is a prostate cancer, prostate carcinoma, or prostate neoplasm.

37. The method of claim 33, wherein the EPS biomarker is a PCA3 RNA or a TMPRSS2:ERG fusion RNA transcript.

38. The method of claim 33, wherein the EPS biomarker is selected from the group consisting of: PSA RNA, TMPRSS2:ERG fusion RNA transcript, copy number of GSTP1 APC, RARβ, or RASSF1 DNA; and TMPRSS2:ERG Type III or VI fusion RNA.

39. The method of claim 33, wherein the method is performed in combination with a digital rectal examination.

40. A method of detecting a prostate cell proliferative disorder in a subject comprising:
(a) obtaining one or more expressed prostatic secretion (EPS) sample from the subject;
(b) measuring serum prostate specific antigen (PSA) levels in the EPS sample of the subject; and
(c) measuring the level of one or more biomarkers from the EPS sample, said biomarkers selected from the group consisting of:
i) PSA RNA;
ii) TMPRSS2:ERG RNA;
iii) copy number of GSTP1 APC, RARβ, or RASSF1 DNA;
iv) TMPRSS2:ERG Type III or VI fusion RNA; and
v) methylated copies of GSTP1, APC, RARβ, PCA3, or RASSF1;
wherein elevated serum PSA levels as compared to normal PSA serum levels and presence of one or more of the biomarkers from the EPS sample indicates that the subject has a prostate cell proliferative disease.

41. The method of claim 40, wherein the method is performed in combination with a digital rectal examination.

42. The method of claim 40, wherein methylated copies are detected using a polymerase chain reaction and wherein, prior to amplifying isolated DNA, the DNA is treated with bisulfite without having been previously denatured.

43. The method of claim 40, wherein the detection of a prostate cell proliferative disorder includes diagnosis and/or grading of a prostatic tumor in a subject.

44. A method of detecting a prostate proliferative cell disorder in a subject by measuring the level of methylation of a panel of target biomarkers in an expressed prostatic secretion sample obtained from the subject, wherein the target biomarkers are GSTP1, APC, RARβ, and RASSF1, and wherein there is a positive correlation between a ratio of methylated DNA to total methylated and unmethylated DNA for the biomarkers and the presence of a prostate proliferative cell disorder.

45. The method of claim 44, further comprising measuring of serum PSA levels in the subject and performing a digital rectal exam on the subject, wherein increased PSA levels as compared to normal levels and an irregular results from a digital rectal examination are additional indicators of the presence of a prostate proliferative cell disorder.

46. The method of claim 44, further comprising measuring biomarker is TMPRSS2:ERG Type III and/or IV fusion RNA in an EPS serum sample, wherein presence of the biomarker is an additional indication of the presence of a prostate proliferative cell disorder.

47. The method of claim 46, wherein TMPRSS2:ERG type III and/or IV is quantified using reverse transcription PCR.
